(19) United States
(12) Patent Application Publication
(10) Pub. No.: US 2021/0253642 A1
(43) Pub. Date: Aug. 19, 2021

(54) CALPAIN MODULATORS AND THERAPEUTIC USES THEREOF BACKGROUND

(71) Applicant: Blade Therapeutics, Inc., South San Francisco, CA (US)

(72) Inventor: Brad Owen Buckman, Oakland, CA (US)

(21) Appl. No.: 17/053,617

(22) PCT Filed: May 7, 2019
(86) PCT No.: PCT/US2019/031180

§ 371 Date: Nov. 6, 2020

Related U.S. Application Data
(60) Provisional application No. 62/669,241, filed on May 9, 2018.

(51) Int. Cl. C07K 11/02; A61K 9/00; A61P 19/04; A61P 11/00; A61P 1/16; A61P 13/12; A61P 17/02

(52) U.S. Cl. CPC C07K 11/02; A61K 9/0019; A61P 19/04; A61K 38/00; A61P 1/16; A61P 13/12; A61P 17/02; A61P 11/00

(57) ABSTRACT

Disclosed herein are small molecule calpain modulators, pharmaceutical compositions, preparation methods and their use as therapeutic agents. The therapeutic agents can be used for treating fibrotic disease or a resulting secondary disease state or condition. The small molecules can inhibit calpain through contact with CAPN1, CAPN2, and/or CAPN9 enzymes.

### BACKGROUND

### Field of the Invention

The present invention relates to the fields of chemistry and medicine. More particularly, the present invention relates to 1-oxa-4,7-diazacyclododec-9-ene-2,5,8-trione based compounds as small molecule calpain modulators, compositions, their preparation, and their use as therapeutic agents.

### Description of the Related Art

Fibrotic disease accounts for an estimated 45% of deaths in the developed world but the development of therapies for such diseases is still in its infancy. The current treatments for fibrotic diseases, such as for idiopathic lung fibrosis, renal fibrosis, systemic sclerosis, and liver cirrhosis, are few in number and only alleviate some of the symptoms of fibrosis while failing to treat the underlying cause.

Despite the current limited understanding of the diverse etiologies responsible for these conditions, similarities in the phenotype of the affected organs, across fibrotic diseases, strongly support the existence of common pathogenic pathways. At present, it is recognized that a primary driver of fibrotic disease is a high transforming growth factor-beta (TGFβ) signaling pathway which can promote the transformation of normally functioning cells into fibrosis-promoting cells. Termed “myofibroblasts,” these transformed cells can secrete large amounts of extracellular matrix proteins and matrix degrading enzymes, resulting in the formation of scar tissue and eventual organ failure. This cellular process is transformative and termed “myofibroblast differentiation” (which includes Epithelial-to-Mesenchymal Transition (EpMT) and its variations like Endothelial-to-Mesenchymal Transition (EnMT) and Fibroblast-to-Myofibroblast Transition (FMT)). This process is a major target for the treatment of fibrotic diseases. Myofibroblast differentiation has also been shown to occur within cancer cells that have been chronically exposed to high TGFβ, causing stationary epithelial cells to become motile, invasive, and metastasize. Thus, within the context of cancer, the signaling has been documented to associate with the acquisition of drug resistance, immune system evasion, and development of stem cell properties.

Despite the tremendous potential of myofibroblast differentiation-inhibiting drugs, and the numerous attempts to develop a working treatment, the data gathered thus far has yet to translate into practical therapy. This is partly due to the lack of an ideal target protein. Initial strategies to target the myofibroblast differentiation process focused on proximal inhibition of the TGFβ signaling pathway by various methods, including targeting ligand activators (e.g. alpha-v integrins), ligand-receptor interactions (e.g., using neutralizing antibodies) or TGFβ receptor kinase activity (e.g., small molecule chemical compound drugs to block signal transduction). Unfortunately, TGFβ is a pleiotropic cytokine with many physiological functions such that global suppression of TGFβ signaling was also associated with severe side effects. Additionally, current data suggests that such proximal inhibition may be vulnerable to pathologic workaround strategies (i.e., due to redundancy or compensation), that would limit the utility of such drugs. Further complicating matters is that, in cancer, TGFβ signaling early on functions as an anti-tumorigenic growth inhibitor but later becomes tumor promoting and is another reason why selective inhibition of pathogenic elements of signaling is so strongly desired. In light of these inherent limitations, current treatment strategies have refocused on identification and inhibition of critical distal events in TGFβ signaling, which in theory would preferentially target the pathologic, but not physiological functions of TGFβ signaling.

### SUMMARY

A compound having the structure of the formula I:

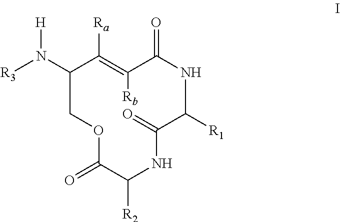

or a pharmaceutically acceptable salt thereof, wherein:

Rₐ and R_{b} are independently selected from —H, optionally substituted C₁₋₈ alkyl, and optionally substituted C₁₋₈ alkoxyakyl;

R₃ is selected from the group consisting of —H, —COOR₃ₐ, —CON(R_{3b})₂, —COC(R₄)₂NH(R₅), optionally substituted C₁₋₄ alkyl, optionally substituted C-s alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R₅ is selected from the group consisting of —H, —COOR₃ₐ, —CON(R_{3b})₂, —COC(R₆)₂NH(R₇), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R₇ is selected from the group consisting of —H, —COOR₃ₐ, —COR_{3b}, —COC(R₄)₂NH(R₅), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R₁, R₂, R₄, and R₆ are independently selected from —H, optionally substituted C₁₋₄ alkyl, and optionally substituted C₁₋₈ alkoxyalkyl, optionally substituted C₆₋₁₀ aryl, optionally substituted 5-10 membered heteroaryl, optionally substituted C₃₋₁₀ carbocyclyl, optionally substituted C₂₋₈ alkenyl, optionally substituted aralkyl wherein the aryl ring in the aralkyl is further optionally substituted with one or more R₈, optionally substituted —O—C₁₋₆ alkyl, optionally substituted —O—C₂₋₆ alkenyl, and any natural or non-natural amino acid side chain;

R₈ is —OSi C₁₋₄ alkyl; and

R₃, and R_{3b} are independently selected from —H, optionally substituted C₁₋₄ alkyl, optionally substituted C₃₋₇ carbocyclyl, optionally substituted aralkyl, optionally substituted C₂₋₁₀ alkenyl, optionally substituted 5-10 membered heterocyclyl, optionally substituted C₆₋₁₀ aryl, and optionally substituted 5-10 membered heteroaryl.

Other embodiments disclosed herein include a pharmaceutical composition comprising a therapeutically effective amount of a compound disclosed herein and a pharmaceutically acceptable excipient.

Other embodiments disclosed herein include a method of treating diseases and conditions mediated at least in part by the physiologic effects of CAPN1, CAPN2, or CAPN9, or combinations thereof, comprising administering to a subject in need thereof a compound disclosed herein.

In some embodiments, compounds disclosed herein are specific inhibitors of one of: CAPN1, CAPN2 or CAPN9.

In some embodiments, compounds disclosed herein are selective inhibitors of one of: CAPN1, CAPN2 or CAPN9.

In some embodiments, compounds disclosed herein are selective inhibitors of: CAPN1 and CAPN2, or CAPN1 and CAPN9, or CAPN2 and CAPN9.

In some embodiments, compounds disclosed herein are effective inhibitors of CAPN1, CAPN2 and/or CAPN9.

In some embodiments, the 1-oxa-4,7-diazacyclododec-9-ene-2,5,8-trione based compounds disclosed herein are broadly effective in treating a host of conditions arising from fibrosis or inflammation, and specifically including those associated with myofibroblast differentiation. Accordingly, compounds disclosed herein are active therapeutics for a diverse set of diseases or disorders that include or that produces a symptom which include, but are not limited to: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders. In other embodiments, the compounds disclosed herein can be used can be used in metabolic and reaction kinetic studies, detection and imaging techniques and radioactive treatments.

In some embodiments, the compounds disclosed herein are used to treat diseases or conditions or that produces a symptom in a subject which include, but not limited to: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases.

In certain embodiments methods are provided for alleviating or ameliorating a condition or disorder, affected at least in part by the enzymatic activity of calpain 1 (CAPN1), calpain 2 (CAPN2), and/or calpain 9 (CAPN9), or mediated at least in part by the enzymatic activity of CAPN1, CAPN2, and/or CAPN9 wherein the condition includes or produces a symptom which includes: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and/or rheumatoid arthritis.

In some embodiments, the methods, compounds, and/or compositions of the present invention are used for prophylactic therapy.

In some embodiments, the CAPN1, CAPN2, and/or CAPN9 inhibiting compounds demonstrate efficacy in animal models of human disease. Specifically, in-vivo treatment of mice, rabbits, and other mammalian subjects with compounds disclosed herein establish the utility of these compounds as therapeutic agents to modulate CAPN1, CAPN2, and/or CAPN9 activities in humans and thereby ameliorate corresponding medical conditions.

Some embodiments provide compounds, pharmaceutical compositions, and methods of use to inhibit myofibroblast differentiation. Some embodiments provide compounds, pharmaceutical compositions, and methods of use for inhibiting CAPN1, CAPN2, and/or CAPN9 or combinations of these enzyme activities such as CAPN1 and CAPN2, or CAPN1 and CAPN9, or CAPN2 and CAPN9. Some embodiments provide methods for treatment of diseases and disorders by inhibiting CAPN1, CAPN2, and/or CAPN9 or combinations of these enzymatic activities.

### DETAILED DESCRIPTION

In some embodiments, compounds that are macrocyclic α-keto amides are provided that act as calpain modulators. Various embodiments of these compounds include compounds having the structures of Formula I as described above or pharmaceutically acceptable salts thereof. The structure of Formula I encompasses all stereoisomers and racemic mixtures, including the following structures and mixtures thereof:

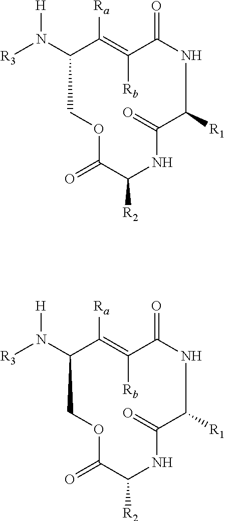

In some embodiments of compounds of Formula (I):

Rₐ and R_{b} are independently selected from —H, optionally substituted C₁₋₈ alkyl, and optionally substituted C₁₋₈ alkoxyalkyl;

R₃ is selected from the group consisting of —H, —COOR₃ₐ, —CON(R_{3b})₂, —COC(R₄)₂NH(R₅), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R₅ is selected from the group consisting of —H, —COOR₃ₐ, —CON(R_{3b})₂, —COC(R₆)₂NH(R₇), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R₇ is selected from the group consisting of —H, —COOR₃ₐ, —COR_{3b}, —COC(R₄)₂NH(R₅), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R₁, R₂, R₄, and R₆ are independently selected from —H, optionally substituted C₁₋₄ alkyl, and optionally substituted C₁₋₈ alkoxyalkyl, optionally substituted C₆₋₁₀ aryl, optionally substituted 5-10 membered heteroaryl, optionally substituted C₃₋₁₀ carbocyclyl, optionally substituted C₂₋₈ alkenyl, optionally substituted aralkyl wherein the aryl ring in the aralkyl is further optionally substituted with one or more R₈, optionally substituted —O—C₁₋₆ alkyl, optionally substituted —O C₂₋₄ alkenyl, and any natural or non-natural amino acid side chain;

R₈ is —OSi C₁₋₄ alkyl; and

R₃ₐ and R_{3b} are independently selected from —H, optionally substituted C₁₋₄ alkyl, optionally substituted C₃₋₇ carbocyclyl, optionally substituted aralkyl, optionally substituted C₂₋₁₀ alkenyl, optionally substituted 5-10 membered heterocyclyl, optionally substituted C₆₋₁₀ aryl, and optionally substituted 5-10 membered heteroaryl.

Some embodiments of compounds of Formula (I) include compounds having the structure of Formula (I-a):

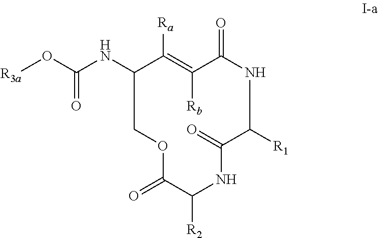

or a pharmaceutically acceptable salt thereof, wherein:

Rₐ and R_{b} are independently selected from —H and optionally substituted C₁₋₈ alkyl;

R₁ and R₂ are independently selected from —H, optionally substituted C₁₋₄ alkyl, optionally substituted C₆₋₁₀ aryl, optionally substituted aralkyl wherein the aryl ring in the aralkyl is further optionally substituted with one or more Rₐ, and any natural or non-natural amino acid side chain; and

R₃ₐ is selected from optionally substituted C₁₋₄ alkyl, optionally substituted aralkyl, and optionally substituted C₆₋₁₀ aryl.

In some embodiments of compounds of Formula (I-a) or their pharmaceutically acceptable salts, R₃ is selected from the group consisting of —H, —COOR₃ₐ, —CON(R_{3b})₂, optionally substituted C₁₋₄ alkyl, optionally substituted C₃₋₇ carbocyclyl, optionally substituted C₆₋₁₀ aryl, and optionally substituted C₆₋₁₀ aryl(C₁-C₆)alkyl.

In some embodiments of compounds of Formula (I-a), R₃ is selected from the group consisting of —H, —COOR₃ₐ, —CON(R_{3b})₂, optionally substituted C₁₋₄ alkyl, and optionally substituted C₆₋₁₀ aryl(C₁-C₆)alkyl.

In some embodiments of compounds of Formula (I-a), R₃ₐ is selected from the group consisting of —H, optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

In some embodiments of compounds of Formula (I-a), R₃ₐ is selected from the group consisting of tert-butyl, methyl, and benzyl.

In some embodiments of compounds of Formula (I-a), R_{3b} is selected from the group consisting of optionally substituted C₁₋₄ alkyl, optionally substituted aralkyl, optionally substituted C₂₋₁₀ alkenyl, and optionally substituted C₆₋₁₀ aryl.

In some embodiments of compounds of Formula (I-a), R_{3b} is selected from the group consisting of methyl, —CH₂CH═CH(CH₂)₅CH₃, and benzyl.

Some embodiments of compounds of Formula (I) include compounds having the structure of Formula (I-b):

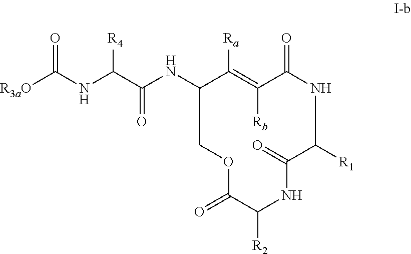

or a pharmaceutically acceptable salt thereof, wherein:

Rₐ and R_{b} are independently selected from —H and optionally substituted C₁₋₈ alkyl;

R₁ R₂, and R₄ are independently selected from —H, optionally substituted C₁₋₄ alkyl, optionally substituted C₆₋₁₀ aryl, optionally substituted aralkyl wherein the aryl ring is further substituted with one or more R₈, and any natural or non-natural amino acid side chain; and

R₃ₐ is selected from optionally substituted C₁₋₄ alkyl, optionally substituted aralkyl, and optionally substituted C₆₋₁₀ aryl.

In some embodiments of compounds of Formula (I-b), R₃ₐ is selected from the group consisting of —H, optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

In some embodiments of compounds of Formula (I-b), R₃ₐ is selected from the group consisting of tert-butyl, methyl, and benzyl.

In some embodiments of compounds of Formulas (I-b), (I-c), and (I-d), R₄ is selected from the group consisting of —H, optionally substituted C₁₋₄ alkyl, optionally substituted C₆₋₁₀ aryl, optionally substituted C₃₋₁₀ carbocyclyl, optionally substituted C₂₋₈ alkenyl, optionally substituted aralkyl wherein the aryl ring is further substituted with one or more R₈.

In some embodiments of compounds of Formulas (I-b), (I-c), and (I-d), R₄ is selected from the group consisting of —H, optionally substituted C₁₋₄ alkyl, and optionally substituted aralkyl wherein the aryl ring is further substituted with one or more R₈.

In some embodiments of compounds of Formulas (I-b), (I-c), and (I-d), R₄ is selected from the group consisting of methyl, isopropyl, isobutyl, benzyl, and p-hydroxybenzyl, and p-methoxybenzyl.

Some embodiments of compounds of Formula (I) include compounds having the structure of Formula (I-c):

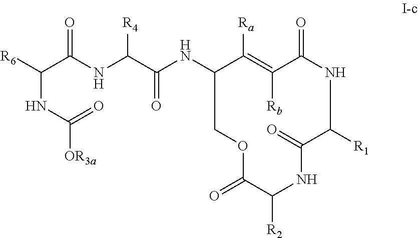

or a pharmaceutically acceptable salt thereof, wherein:

Rₐ and R_{b} are independently selected from —H and optionally substituted C₁₋₈ alkyl;

R₁ R₂, and R₄ are independently selected from —H, optionally substituted C₁₋₄ alkyl, optionally substituted C₆₋₁₀ aryl, optionally substituted aralkyl wherein the aryl ring is further substituted with one or more R₈, and any natural or non-natural amino acid side chain; and

R₃ₐ is selected from optionally substituted C₁₋₄ alkyl, optionally substituted aralkyl, and optionally substituted C₆₋₁₀ aryl.

In some embodiments of compounds of Formulas (I), and (I-c), R₃ₐ is selected from the group consisting of —H, optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

In some embodiments of compounds of Formulas (I), and (I-c), wherein R₃ₐ is selected from the group consisting of tert-butyl, methyl, and benzyl.

Some embodiments of compounds of Formula (I) include compounds having the structure of Formula (I-d):

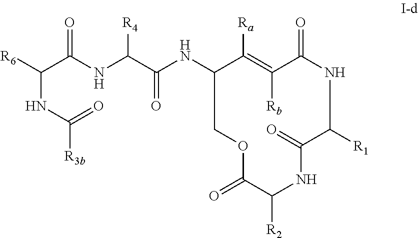

or a pharmaceutically acceptable salt thereof, wherein:

Rₐ and R_{b} are independently selected from —H and optionally substituted C-s alkyl;

R₁ R₂, R₄, and R₆ are independently selected from —H, optionally substituted C₁₋₄ alkyl, optionally substituted C₆₋₁₀ aryl, optionally substituted aralkyl wherein the aryl ring is further substituted with one or more R₈, and any natural or non-natural amino acid side chain; and

R_{3b} is selected from optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

In some embodiments of compounds of Formulas (I), and (I-d), R_{3b} is selected from the group consisting of optionally substituted C₁₋₄ alkyl, optionally substituted aralkyl, optionally substituted C₂₋₁₀ alkenyl, and optionally substituted C₆₋₁₀ aryl.

In some embodiments of compounds of Formulas (I), and (I-d), R_{3b} is selected from the group consisting of methyl, —CH₂CH═CH(CH₂)₅CH₃, and benzyl.

In some embodiments of compounds of Formulas (I) and (I-d), R₆ is selected from the group consisting of —H, optionally substituted C₁₋₄ alkyl, optionally substituted C₆₋₁₀ aryl, optionally substituted C₃₋₁₀ carbocyclyl, optionally substituted C₂₋₈ alkenyl, optionally substituted aralkyl wherein the aryl ring is further substituted with one or more R₈.

In some embodiments of compounds of Formulas (I) and (I-d), R₆ is selected from the group consisting of —H, optionally substituted Cia alkyl, and optionally substituted aralkyl wherein the aryl ring is further substituted with one or more R₈.

In some embodiments of compounds of Formulas (I) and (I-d), R₆ is selected from the group consisting of methyl, isopropyl, isobutyl, benzyl, and p-hydroxybenzyl, and p-methoxybenzyl.

In some embodiments of compounds of Formulas (I), (I-a), (I-b), (I-c), and (I-d), R₁ and R₂ are independently selected from —H, optionally substituted C₁₋₄ alkyl, optionally substituted C₆₋₁₀ aryl, optionally substituted C₃₋₁₀ carbocyclyl, optionally substituted C₂₋₈ alkenyl, optionally substituted aralkyl wherein the aryl ring is further optionally substituted with one or more R₈.

In some embodiments of compounds of Formulas (I), (I-a), (I-b), (I-c), and (I-d), R₁ and R₂ are independently selected from —H, optionally substituted C₁₋₄ alkyl, and optionally substituted aralkyl wherein the aryl ring is further optionally substituted with one or more R₈.

In some embodiments of compounds of Formulas (I), (I-a), (I-b), (I-c), and (I-d), R₁ and R₂ are independently selected from the group consisting of methyl, isopropyl, isobutyl, benzyl, and p-hydroxybenzyl, and p-methoxybenzyl.

In some embodiments of compounds of Formulas (I), (I-a), (I-b), (I-c), and (I-d), R₈ is —OSi C₁₋₄ alkyl.

In some embodiments of compounds of Formulas (I), (I-a), (I-b), (I-c), and (I-d), R₈ is selected from the group consisting of OSiMe₃ and OSi^{t}BuMe₂.

In some embodiments of compounds of Formulas (I), (I-a), (I-b), (I-c), and (I-d), Rₐ and R_{b} are independently selected from —H and optionally substituted C₁₋₈ alkyl.

In some embodiments of compounds of Formulas (I), (I-a), (I-b), (I-c), and (I-d), Rₐ and R_{b} are —H.

Some embodiments include a compound selected from the group consisting of:

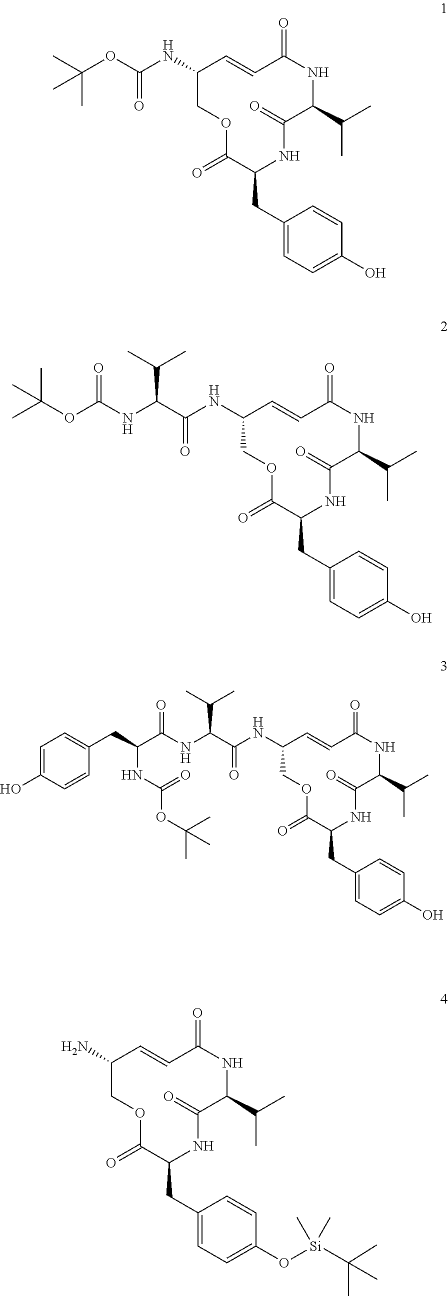 [SMILES: CC(C)[C@@H]1NC(=O)/C=C/[C@@H](NC(=O)OC(C)(C)C)COC(=O)[C@H](Cc2ccc(O)cc2)NC1=O ; CC(C)[C@@H]1NC(=O)/C=C/[C@@H](NC(=O)[C@@H](NC(=O)OC(C)(C)C)C(C)C)COC(=O)[C@H](Cc2ccc(O)cc2)NC1=O ; CC(C)[C@@H]1NC(=O)/C=C/[C@@H](NC(=O)[C@@H](NC(=O)[C@H](Cc2ccc(O)cc2)NC(=O)OC(C)(C)C)C(C)C)COC(=O)[C@H](Cc2ccc(O)cc2)NC1=O ; CC(C)[C@@H]1NC(=O)/C=C/[C@@H](N)COC(=O)[C@H](Cc2ccc(O[Si](C)(C)C(C)(C)C)cc2)NC1=O]

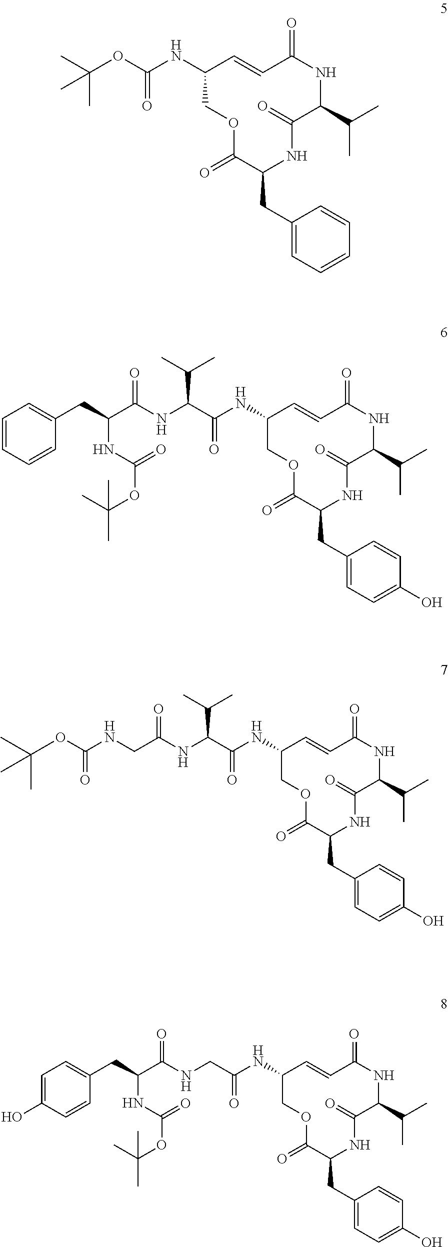

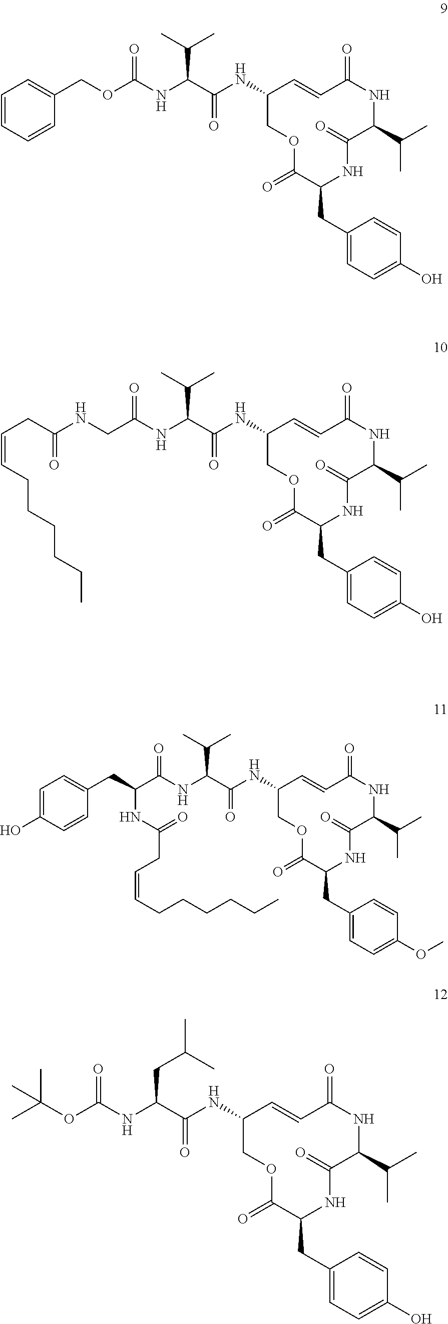

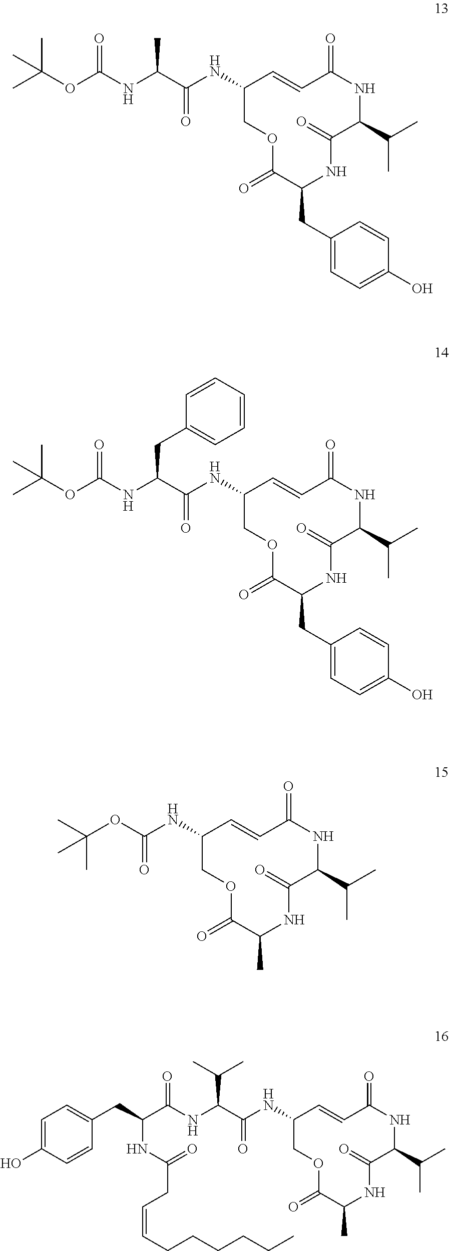 [SMILES: CC(C)[C@@H]1NC(=O)/C=C/[C@@H](NC(=O)[C@H](C)NC(=O)OC(C)(C)C)COC(=O)[C@H](Cc2ccc(O)cc2)NC1=O ; CC(C)[C@@H]1NC(=O)/C=C/[C@@H](NC(=O)[C@H](Cc2ccccc2)NC(=O)OC(C)(C)C)COC(=O)[C@H](Cc2ccc(O)cc2)NC1=O ; CC(C)[C@@H]1NC(=O)/C=C/[C@@H](NC(=O)OC(C)(C)C)COC(=O)[C@H](C)NC1=O ; CCCCCC/C=C\CC(=O)N[C@@H](Cc1ccc(O)cc1)C(=O)N[C@H](C(=O)N[C@@H]1/C=C/C(=O)N[C@@H](C(C)C)C(=O)N[C@@H](C)C(=O)OC1)C(C)C]

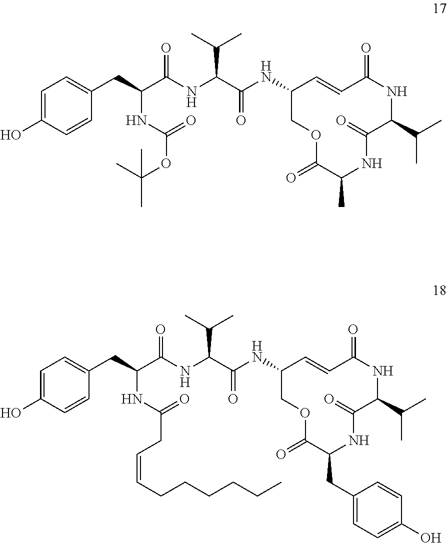 [SMILES: CC(C)[C@@H]1NC(=O)/C=C/[C@@H](NC(=O)[C@@H](NC(=O)[C@H](Cc2ccc(O)cc2)NC(=O)OC(C)(C)C)C(C)C)COC(=O)[C@H](C)NC1=O ; CCCCCC/C=C\CC(=O)N[C@@H](Cc1ccc(O)cc1)C(=O)N[C@H](C(=O)N[C@@H]1/C=C/C(=O)N[C@@H](C(C)C)C(=O)N[C@@H](Cc2ccc(O)cc2)C(=O)OC1)C(C)C]

or pharmaceutically acceptable salts thereof.

Various embodiments include the S-enantiomer, the R-enantiomer, or the racemate at each stereocenter of the above compounds.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopically-Labeled Compounds

Isotopes may be present in the compounds described. Each chemical element as represented in a compound structure may include any isotope of said element. The isotopes may be isotopes of carbon, chlorine, fluorine, hydrogen, iodine, nitrogen, oxygen, phosphorous, sulfur, and technetium, including ¹¹C, ¹³C, ¹⁴C, ³⁶Cl, ¹⁸F, ²H, ³H, ¹²³I, ¹²⁵I, ¹³N, ¹⁵N, ¹⁵O, ¹⁷O, ¹⁸O, ³¹P, ³²P, ³⁵S, and ^{99m}Tc. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise. Isotopically-labeled compounds of the present embodiments are useful in drug and substrate tissue distribution and target occupancy assays. For example, isotopically labeled compounds are particularly useful in SPECT (single photon emission computed tomography) and in PET (positron emission tomography), as discussed further herein.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO- and RS-, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazolylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R₃” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, 1 alkyl, C₂ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₄ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₄ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

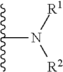

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

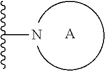

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

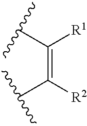

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

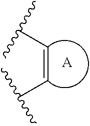

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

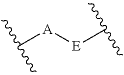

includes the substituent being oriented such that the A is attached at the leftmost attachment point of the molecule as well as the case in which A is attached at the rightmost attachment point of the molecule.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemee or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Synthesis of Compounds of Formula I

In one embodiment, the method involves reacting oxazolidin-4-yl intermediate 1K with an appropriately substituted intermediate (II) under amide coupling conditions to yield the oxazolidin-4-yl intermediate (III). This intermediate was treated with bismuth(III) chloride to hydrolyze the oxazolidin-4-yl ring to yield the intermediate (IV) which was subjected to amide coupling conditions with intermediate (V) to yield the bisallyloxy protected intermediate (VI). The bisallyloxy intermediate (VI) was subjected to treatment with phenylsilane in presence of Pd(0) catalyst to remove the allyloxy groups resulting in the intermediate (VII). The intermediate (VII) was subjected to intramolecular cyclization using pentafluorophenyl diphenylphosphinate (FDPP) to yield the cyclic derivative (VIII) which was subjected to oxidative elimination using sodium periodate to yield the product I-a-1 (Scheme 1).

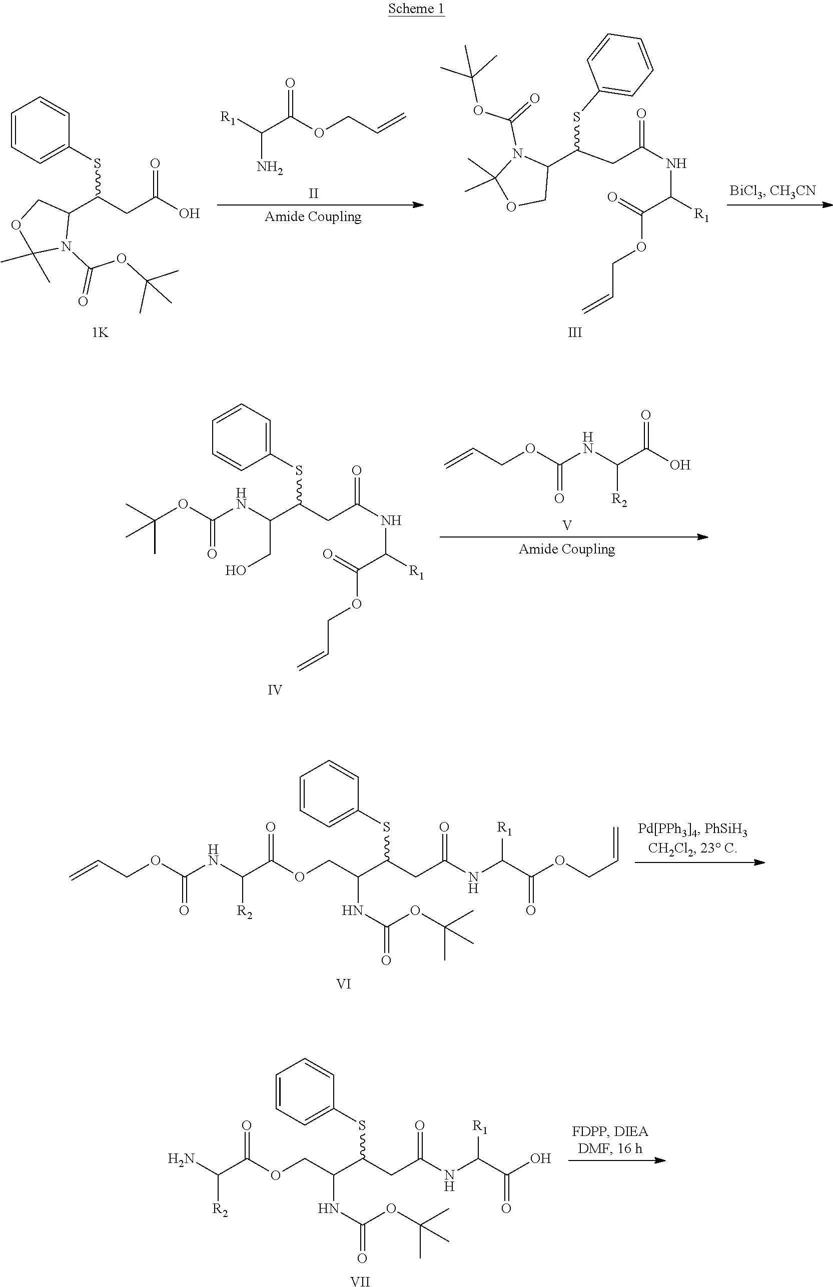

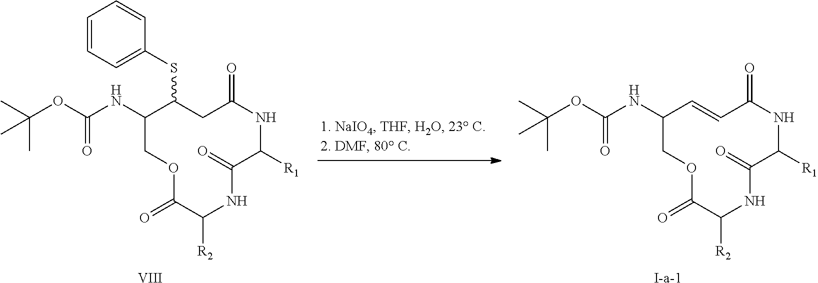

Compound (I-a-1) was subjected to BOC-removal using TFA to yield intermediate (IX) followed by amide coupling with succinimide derivative (X) to yield the corresponding product (I-b-1) as shown in Scheme 2.

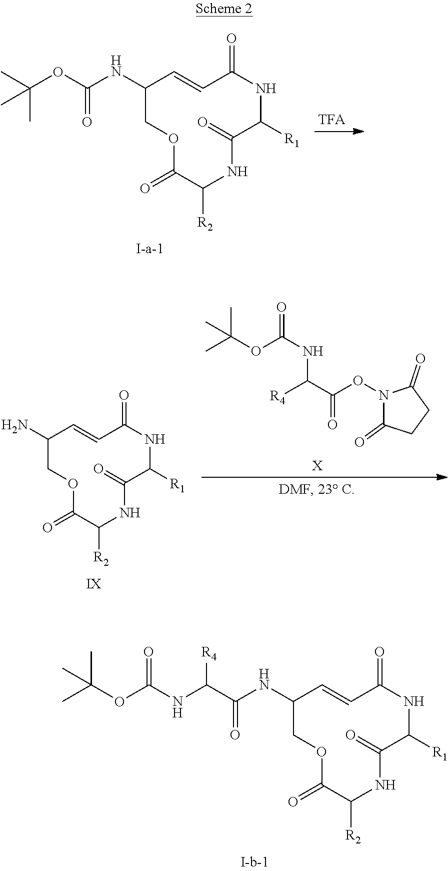

Compound (I-b-1) was subjected to BOC-removal followed by amide coupling to yield product (I-c-1). Similarly product I-c-1 was subjected to BOC-removal followed by amide coupling with carboxylic acid represented by (XI) to yield product (I-d). This synthetic route is generally shown in Scheme 3.

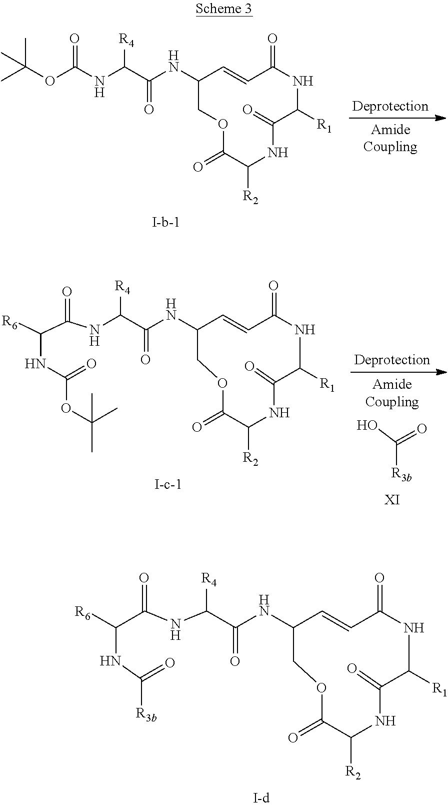

The above example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

### Uses of Isotopically-Labeled Compounds

Some embodiments provide a method of using isotopically labeled compounds and prodrugs of the present disclosure in: (i) metabolic studies (preferably with ¹⁴C), reaction kinetic studies (with, for example 2H or 3H); (ii) detection or imaging techniques [such as positron emission tomography (PET) or single-photon emission computed tomography (SPECT)] including drug or substrate tissue distribution assays; or (iii) in radioactive treatment of patients.

Isotopically labeled compounds and prodrugs of the embodiments thereof can generally be prepared by carrying out the procedures disclosed in the schemes or in the examples and preparations described below by substituting a readily available isotopically labeled reagent for a non-isotopically labeled reagent. An ¹⁸F or ¹¹C labeled compound may be particularly preferred for PET, and an ¹²³I labeled compound may be particularly preferred for SPECT studies. Further substitution with heavier isotopes such as deuterium (i.e., ²H) may afford certain therapeutic advantages resulting from greater metabolic stability, for example increased in vivo half-life or reduced dosage requirements.

Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarily, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et ah (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable tor administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable earners include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Liebemian et al, Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HQ, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al, Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 99.99 wt % of a compound of the present technology based on the total formulation, with the balance being one or more suitable pharmaceutical excipients. Preferably, the compound is present at a level of about 1 80 wt %. Representative pharmaceutical formulations are described below.

### FORMULATION EXAMPLES

The following are representative pharmaceutical formulations containing a compound of Formula I.

### Formulation Example 1—Tablet Formulation

The following ingredients are mixed intimately and pressed into single scored tablets.

| | Quantity per |
|---|---|
| Ingredient | tablet, mg |
| Compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| Compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| Compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4—Injectable formulation

The following ingredients are mixed to form an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| Compounds disclosed herein | 500 mg |
| Witepsol ® H-15 | balance |

### Methods of Treatment

The compounds disclosed herein or their tautomers and/or pharmaceutically acceptable salts thereof can effectively act as CAPN1, CAPN2, and/or CAPN9 inhibitors and treat conditions affected at least in part by CAPN1, CAPN2, and/or CAPN9. Some embodiments provide pharmaceutical compositions comprising one or more compounds disclosed herein and a pharmaceutically acceptable excipient. Some embodiments provide a method for treating a fibrotic disease with an effective amount of one or more compounds as disclosed herein.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

Some embodiments include combinations of a compound, composition or pharmaceutical composition described herein with any other pharmaceutical compound approved for treating fibrotic or myofibroblast differentiation associated diseases or disorders.

Some embodiments provide a method for inhibiting CAPN1, CAPN2, and/or CAPN9 and/or a method for treating a disease affected at least in part by CAPN1, CAPN2, and/or CAPN9 with an effective amount of one or more compounds as disclosed herein.

The compounds disclosed herein are useful in inhibiting CAPN1, CAPN2, and/or CAPN9 enzymes and/or treating disorders relating to fibrosis or myofibroblast differentiation.

Some embodiments provide a method for inhibiting CAPN1, CAPN2, and/or CAPN9 which method comprises contacting cells (including neurons/microglia/invading macrophages) with an effective amount of one or more compounds as disclosed herein.

Some embodiments provide a method for treating a fibrotic disease, which method comprises administering to a subject an effective amount of one or more compounds or a pharmaceutical composition disclosed herein comprising a pharmaceutically acceptable excipient.

Some embodiments provide a method for treating a disease affected at least in part by CAPN1, CAPN2, and/or CAPN9, which method comprises administering to a subject an effective amount of one or more compounds or a pharmaceutical composition disclosed herein comprising a pharmaceutically acceptable excipient.

Some embodiments provide a method for inhibiting CAPN1, CAPN2, and/or CAPN9 is provided wherein the method comprises contacting cells with an effective amount of one or more compounds disclosed herein. In some embodiments a method for inhibiting CAPN1, CAPN2, and/or CAPN9 is performed in-vitro or in-vivo.

Calpains are also expressed in ceils other than neurons, microglia and invading macrophages. In particular, they are important in skeletal muscle and herein inhibition of calpains also refers to inhibition in these cells as well.

### Selective Inhibition

Some embodiments provide a method for competitive binding with calpastatin (CAST), the method comprising contacting a compound disclosed herein with CAPN1, CAPN2, and/or CAPN9 enzymes residing inside a subject. In such a method, the compound specifically inhibits one or more of the enzymes selected from the group consisting of: CAPN1, CAPN2, and CAPN9 by at least 2-fold, by at least 3-fold, by at least 4-fold, by at least 5-fold, by at least 10-fold, by at least 15-fold, by at least 20-fold, by at least 50-fold, by at least 100-fold, by at least 150-fold, by at least 200-fold, by at least 400-fold, or by at least 500-fold.

Some embodiments provide a method for selectively inhibiting CAPN1 in the presence of CAPN2 and CAPN9, which includes contacting cells (including neurons/microglia/invading macrophages) with an effective amount of one or more compounds disclosed herein.

Some embodiments provide a method for selectively inhibiting CAPN2 in the presence of CAPN1 and CAPN9, winch includes contacting cells (including neurons/microglia/invading macrophages) with an effective amount of one or more compounds disclosed herein.

Some embodiments provide a method for selectively inhibiting CAPN9 in the presence of CAPN2 and CAPN1, which includes contacting cells (including neurons/microglia/invading macrophages) with an effective amount of one or more compounds disclosed herein.

Some embodiments provide a method for selectively inhibiting CAPN1 and CAPN2 in the presence of CAPN9, which includes contacting cells (including neurons/microglia/invading macrophages) with an effective amount of one or more compounds disclosed herein.

Some embodiments provide a method for selectively inhibiting CAPN1 and CAPN9 in the presence of CAPN2, which includes contacting cells (including neurons/microglia/invading macrophages) with an effective amount of one or more compounds disclosed herein.

Some embodiments provide a method for selectively inhibiting CAPN2 and CAPN9 in the presence of CAPN1, which includes contacting cells (including neurons/microglia/invading macrophages) with an effective amount of one or more compounds disclosed herein.

Some embodiments provide a method for treating a fibrotic disease, which method comprises administering to a subject an effective amount of one or more compounds which specifically inhibits CAPN1, CAPN2, and/or CAPN9, said compounds or a pharmaceutical composition comprising one or more compounds disclosed herein and a pharmaceutically acceptable excipient.

Some embodiments provide a method for treating a disease affected at least in part by CAPN1, CAPN2, and/or CAPN9, which method comprises administering to a subject an effective amount of one or more compounds which specifically inhibits CAPN1, CAPN2, and/or CAPN9, said compounds being selected from compounds disclosed herein or a pharmaceutical composition comprising one or more compounds disclosed herein and a pharmaceutically acceptable excipient.

Some embodiments provide a method for treating a fibrotic disease, which method comprises administering to a subject an effective amount of one or more compounds which selectively inhibits CAPN1, CAPN2, and/or CAPN9, said compounds being selected from compounds disclosed herein or a pharmaceutical composition comprising one or more compounds disclosed herein and a pharmaceutically acceptable excipient.

Some embodiments provide a method for treating a disease affected at least in part by CAPN1, CAPN2, and/or CAPN9, which method comprises administering to a subject an effective amount of one or more compounds which selectively inhibits CAPN1, CAPN2, and/or CAPN9, said compounds being selected from compounds disclosed herein or a pharmaceutical composition comprising one or more compounds disclosed herein and a pharmaceutically acceptable excipient.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:5.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:10.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:20.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:50.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:100.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:200.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:250.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:500.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:5.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:10.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:20.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:50.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:100.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:200.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:250.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:500.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:5.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:10.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:20.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:50.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:100.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:200.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:250.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:500.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:5.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:10.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:20.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:50.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:100.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:200.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:250.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:500.

Some embodiments provide a method for prophylactic therapy or treatment of a subject having a fibrotic disorder wherein said method comprising administering an effective amount of one or more compounds disclosed herein to the subject in need thereof.

Some embodiments provide a method for prophylactic therapy or treatment of a subject having a disorder affected by CAPN1, CAPN2, and/or CAPN9 wherein said method comprising administering an effective amount of one or more compounds disclosed herein to the subject in need thereof.

Some embodiments provide a method for inhibiting myofibroblast differentiation (e.g., Epithelial/Endothelial-to-Mesenchymal Transition (EpMT/EnMT)) is provided wherein the method comprises contacting cells with an effective amount of one or more compounds disclosed herein. In one aspect, the method for inhibiting myofibroblast differentiation (e.g., Epithelial/Endothelial-to-Mesenchymal Transition (EpMT/EnMT)) is performed in-vitro or in-vivo.

Some embodiments provide a method for treating a disease or condition selected from the group consisting of or that produces a symptom selected from the group consisting of: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases, wherein which method comprises administering to a subject an effective amount of one or more compounds disclosed herein to a subject in need thereof.

Some embodiments provide a method for treating liver fibrosis.

Some embodiments provide a method for treating cardiac fibrosis. Some embodiments provide a method for treating fibrosis in rheumatoid arthritis diseases.

Some embodiments provide a method for treating a condition affected by CAPN1, CAPN2, and/or CAPN9, which is in both a therapeutic and prophylactic setting for subjects. Both methods comprise administering of one or more compounds disclosed herein to a subject in need thereof.

Some embodiments provide a method for treating stiff skin syndrome.

Preferred embodiments include combinations of a compound, composition or pharmaceutical composition described herein with other CAPN1, CAPN2, and/or CAPN9 inhibitor agents, such as anti-CAPN1, CAPN2, AND/OR CAPN9 antibodies or antibody fragments, CAPN1, CAPN2, and/or CAPN9 antisense, iRNA, or other small molecule CAPN1, CAPN2, and/or CAPN9 inhibitors.

Some embodiments include combinations of a compound, composition or pharmaceutical composition described herein to inhibit myofibroblast differentiation (e.g., Epithelial/Endothelial-to-Mesenchymal Transition (EpMT/EnMT)).

Some embodiments include combinations of one or more of these compounds which are inhibitors of one or more (or all three) CAPN1, CAPN2, and/or CAPN9, alone or in combination with other TGFβ signaling inhibitors, could be used to treat or protect against or reduce a symptom of a fibrotic, sclerotic or post inflammatory disease or condition including: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, postvasectomy pain syndrome, and rheumatoid arthritis.

Some embodiments include a combination of the compounds, compositions and/or pharmaceutical compositions described herein with an additional agent, such as anti-inflammatories including glucocorticoids, analgesics (e.g. ibuprofen), aspirin, and agents that modulate a Th2-immune response, immunosuppressants including methotrexate, mycophenolate, cyclophosphamide, cyclosporine, thalidomide, pomalidomide, leflunomide, hydroxychloroquine, azathioprine, soluble bovine cartilage, vasodilators including endothelin receptor antagonists, prostacyclin analogues, nifedipine, and sildenafil, IL-6 receptor antagonists, selective and non-selective tyrosine kinase inhibitors, Wnt-pathway modulators, PPAR activators, caspase-3 inhibitors, LPA receptor antagonists, B cell depleting agents, CCR2 antagonists, pirfenidone, cannabinoid receptor agonists, ROCK inhibitors, miRNA-targeting agents, toll-like receptor antagonists, CTGF-targeting agents, NADPH oxidase inhibitors, tryptase inhibitors, TGFD inhibitors, relaxin receptor agonists, and autologous adipose derived regenerative cells.

### Indications

In some embodiments, the compounds and compositions comprising the compounds described herein can be used to treat a host of conditions arising from fibrosis or inflammation, and specifically including those associated with myofibroblast differentiation. Example conditions include liver fibrosis (alcoholic, viral, autoimmune, metabolic and hereditary chronic disease), renal fibrosis (e.g., resulting from chronic inflammation, infections or type II diabetes), lung fibrosis (idiopathic or resulting from environmental insults including toxic particles, sarcoidosis, asbestosis, hypersensitivity pneumonitis, bacterial infections including tuberculosis, medicines, etc.), interstitial fibrosis, systemic scleroderma (autoimmune disease in which many organs become fibrotic), macular degeneration (fibrotic disease of the eye), pancreatic fibrosis (resulting from, tor example, alcohol abuse and chronic inflammatory disease of the pancreas), fibrosis of the spleen (from sickle ceil anemia, other blood disorders), cardiac fibrosis (resulting from infection, inflammation and hypertrophy), mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic reperfusion injury associated fibrosis, injection fibrosis, cirrhosis, diffuse parenchymal lung disease, post-vasectomy pain syndrome, and rheumatoid arthritis diseases or disorders.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### EXAMPLES

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
DCM=dichloromethane
DIEA=N,N-Diisopropylethylamine
DIPEA=N,N-Diisopropylethylamine
DMF=N,N-dimethylformamide
DMP=Dess Martin Periodinane
DNs=dinitrosulfonyl
ESBL=extended-spectrum β-lactamase
EtOAc=ethyl acetate
EA=ethyl acetate
FCC=Flash Column Chromatography
FDPP=Pentaflurophenyl diphenylphosphinate
HATU=2-(7-aza-1H-benzotriazole-1-yl)-1,1,3,3-tetramethyluronium hexafluorophosphate
MeCN=acetonitrile
NMR=nuclear magnetic resonance
PE=Petroleum Ether
Prep=preparatory
Py=pyridine
Sat.=saturated aqueous
TBDMSCl=tert-butyldimethylsilyl chloride
TBS=tert-butyldimethylsilyl
TFA=trifluoroacetic acid
THF=tetrahydrofuran
TLC=thin layer chromatography

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### Compounds 1, 4, and 2

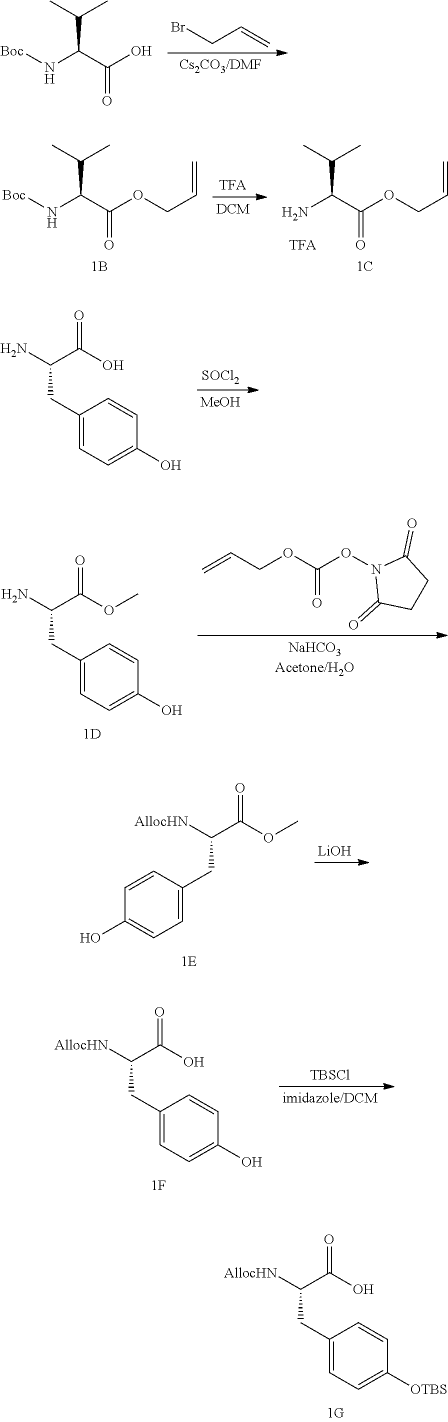

To a solution of (tert-butoxycarbonyl)-L-valine (20 g, 92.05 mmol) and Cs₂CO₃ (74.98 g, 230.14 mmol) in DMF (300 mL) was added 3-bromoprop-1-ene (22.27 g, 184.11 mmol) dropwise, the mixture was stirred at 10° C. for 20 h. The solid was filtered, the filtrate was diluted with H₂O (400 mL), extracted with EtOAc (100 mL×2). The organics were collected and concentrated to give a residue. The residue was purified by flash silica gel chromatography (ISCO®; 220 g SepaFlash® Silica Flash Column, Eluent of 0˜10% Ethyl acetate/Petroleum ether gradient @ 100 mL/min). Compound 1B (17 g, yield: 71.8%) as colorless oil was obtained. ¹H NMR (400 MHz, CDCl₃) δ 5.96-5.86 (m, 1H), 5.34 (dd, J=1.5, 17.1 Hz, 1H), 5.25 (d, J=10.5 Hz, 1H), 5.02 (d, J=8.8 Hz, 1H), 4.70-4.56 (m, 2H), 4.24 (dd, J=4.6, 9.0 Hz, 1H), 2.23-2.08 (m, 1H), 1.44 (s, 9H), 0.96 (d, J=6.8 Hz, 3H), 0.89 (d, J=7.1 Hz, 3H).

To a solution of compound 1B (17 g, 66.06 mmol) in DCM (100 mL) was added TFA (574 mmol, 42.5 mL), the mixture was stirred at 15° C. for 4 h. The reaction mixture was concentrated to give a residue. Compound 1C (20 g, crude, TFA) was obtained as light yellow oil, which was used into the next step without further purification. ¹H NMR (400 MHz, CDCl₃) δ 11.24-9.99 (m, 1H), 8.04 (s, 2H), 5.94-5.84 (m, 1H), 5.46-5.18 (m, 2H), 4.83-4.44 (m, 2H), 3.97 (d, J=4.2 Hz, 1H), 2.40-2.34 (m, 1H), 1.06 (dd, J=1.0, 6.8 Hz, 6H).

To a solution of Z-tyrosine (30 g, 165.57 mmol) in MeOH (500 mL) at 0° C. was added SOCl₂ (39.4 g, 331.15 mmol) dropwise. After addition, the mixture was warmed up to 25° C. and stirred for 12 h. The solvent was removed in vacuo. The residue was triturated with TBME (500 mL). The solid was filtered, collected and dried in vacuo to give compound 1D (32 g, yield: 99.0%) as white solid.

To a solution of compound 1D (32 g, 163.92 mmol) and NaHCO₃ (34.43 g, 409.80 mmol) in Acetone (150 mL) and H₂O (150 mL) was added allyl (2,5-dioxopyrrolidin-1-yl) carbonate (35.91 g, 180.31 mmol). The mixture was stirred at 25° C. for 12 h. The mixture was adjusted to pH 3 with 1N HCl. The organic solvent was removed in vacuo. The aqueous phase was extracted with EtOAc (300 mL×2). The organics were collected and concentrated in vacuo. The residue was purified by column chromatography (SiO₂, Petroleum ether/Ethyl acetate=5/1 to 1/1). Compound 1E (44 g, 73.3% yield) was obtained as a colorless oil. ¹H NMR (400 MHz, DMSO-d₆) δ 9.21 (s, 1H), 7.68 (d, J=8.0 Hz, 1H), 7.00 (br d, J=8.4 Hz, 2H), 6.64 (d, J=8.4 Hz, 2H), 5.88-5.77 (m, 1H), 5.24-5.17 (m, 1H), 5.15-5.10 (m, 1H), 4.42-4.36 (m, 2H), 4.16-4.08 (m, 1H), 3.58 (s, 3H), 2.92-2.84 (m, 1H), 2.77-2.67 (m, 1H). MS (ESI) m/z (M+H)⁺ 280.1.

To a solution of compound 1E (34 g, 121.74 mmol) in THF (150 mL) and H₂O (150 mL) was added LiOH.H₂O (12.77 g, 304.35 mmol). The mixture was stirred at 0° C. for 1 h. The mixture was extracted with cold TBME (200 mL×2), the water phase was added HCl (1M) until pH 3, then the mixture was extracted with EA (500 mL×2), the organic layer was washed with brine (500 mL×2), dried over Na₂SO₄, filtered and concentrated. Compound 1F (30 g, 92.9% yield) was obtained as a colorless oil. ¹H NMR (400 MHz, DMSO-d₆) δ 12.58 (br s, 1H), 9.18 (br s, 1H), 7.48 (d, J=8.4 Hz, 1H), 7.01 (d, J=8.4 Hz, 2H), 6.62 (d, J=8.4 Hz, 2H), 5.88-5.76 (m, 1H), 5.24-5.17 (m, 1H), 5.16-5.08 (m, 1H), 4.44-4.32 (m, 2H), 4.07-4.02 (m, 1H), 2.93-2.86 (m, 1H), 2.72-2.63 (m, 1H).

To a solution of compound 1F (30 g, 113.10 mmol) in DCM (750 mL) was added Imidazole (23.10 g, 339.29 mmol) and TBSCl (37.50 g, 248.81 mmol) at 0° C. The mixture was stirred at 25° C. for 12 h. The mixture was concentrated, then THF (300 mL), H₂O (600 mL), K₂CO₃ (7.0 g) were added and the mixture was stirred for 1.5 h. The mixture was added HCl (1M) until pH 3, extracted with EA (500 mL×2), the organic layer was washed with brine (500 mL), dried over Na₂SO₄, filtered and concentrated. The residue was purified by column chromatography (SiO₂, Petroleum ether/Ethyl acetate=3/1 to 1/1, with 1% HCOOH). Compound 1G (44 g, crude) was obtained as a yellow oil. ¹H NMR (400 MHz, DMSO-d₆) δ 12.71 (br s, 1H), 7.56 (d, J=8.8 Hz, 1H), 7.17-7.10 (m, 2H), 6.77-6.72 (m, 2H), 5.90-5.79 (m, 1H), 5.25-5.18 (m, 1H), 5.17-5.09 (m, 1H), 4.45-4.36 (m, 2H), 4.15-4.07 (m, 1H), 3.03-2.94 (m, 1H), 2.80-2.71 (m, 1H), 0.94 (s, 9H), 0.17 (s, 6H). MS (ESI) m/z (M+H)⁺ 380.2.

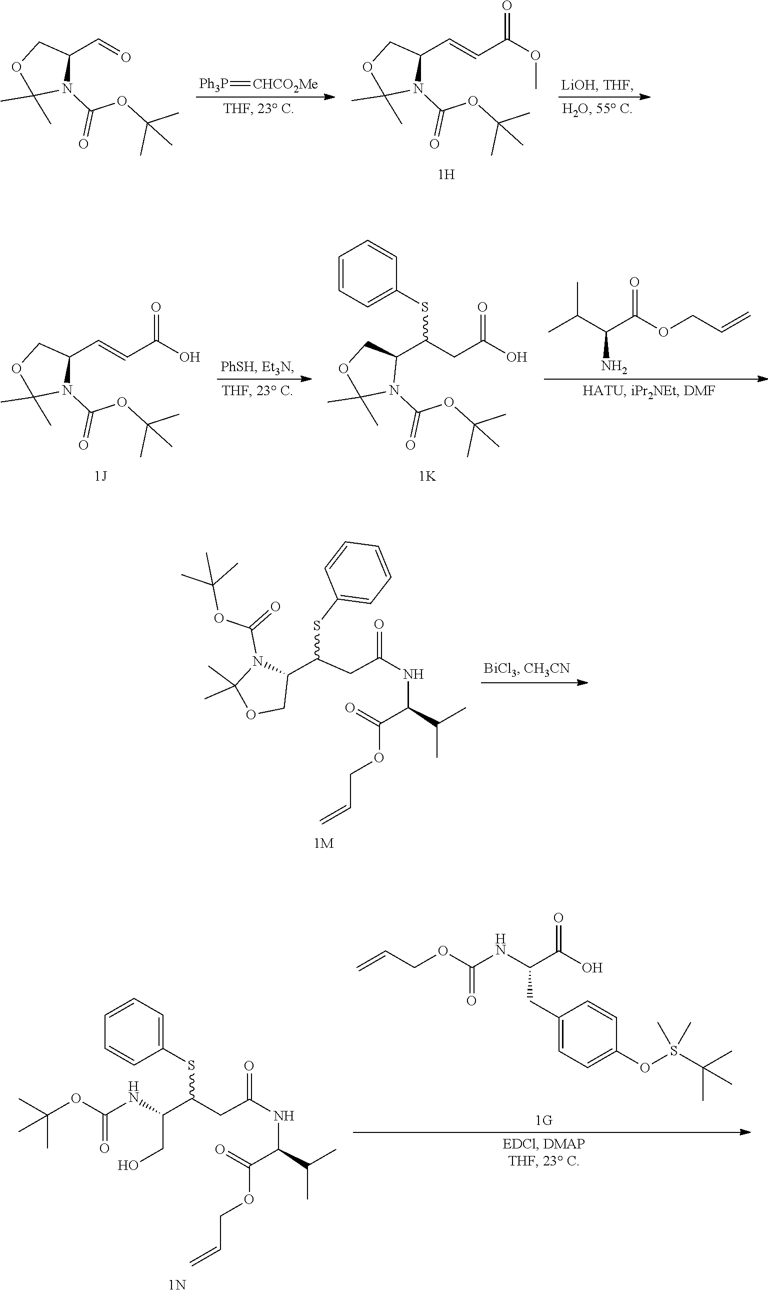

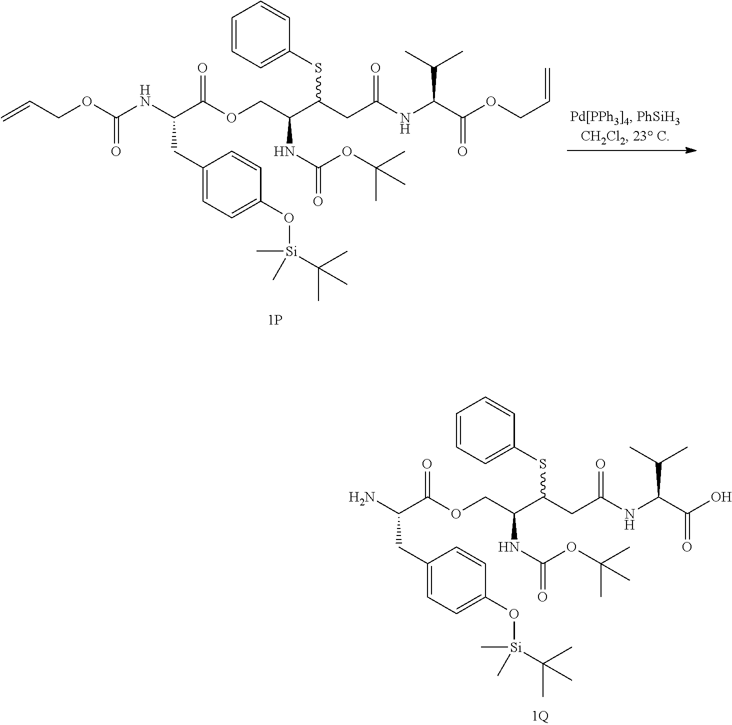 [SMILES: C=CCOC(=O)N[C@@H](Cc1ccc(O[Si](C)(C)C(C)(C)C)cc1)C(=O)OC[C@H](NC(=O)OC(C)(C)C)C(CC(=O)N[C@H](C(=O)OCC=C)C(C)C)Sc1ccccc1 ; CC(C)[C@H](NC(=O)CC(Sc1ccccc1)[C@H](COC(=O)[C@@H](N)Cc1ccc(O[Si](C)(C)C(C)(C)C)cc1)NC(=O)OC(C)(C)C)C(=O)O]

To a solution of tert-butyl (S)-4-formyl-2,2-dimethyloxazolidine-3-carboxylate (15 g, 65.42 mmol) in THF (300 mL) was added methyl 2-(triphenyl-phosphanylidene)acetate (24.06 g, 71.97 mmol) at 25° C. and the reaction mixture was stirred at this temperature for 16 h, the solvent was evaporated and the residue in EA (300 mL) was stirred for 10 min, filtered, the filtrate was concentrated to give a residue. The residue was purified by flash silica gel chromatography (ISCO®; 220 g SepaFlash® Silica Flash Column, Eluent of 010%20% Ethyl acetate/Petroleum ether gradient @100 mL/min). Compound 1H (30 g, yield: 80.4%) was obtained as a colorless oil. (two batches), ¹H NMR (400 MHz, DMSO-Je) δ 6.78 (dd, J=7.0, 15.3 Hz, 1H), 5.87 (br d, J=15.6 Hz, 1H), 4.59-4.43 (m, 1H), 4.14-4.02 (m, 1H), 3.80 (dd, J=1.6, 9.2 Hz, 1H), 3.68 (s, 3H), 1.51 (s, 3H), 1.43 (br d, J=5.0 Hz, 6H), 1.35 (s, 6H).

To a solution of compound 1H (20 g, 70.09 mmol) in THF (300 mL) was added LiOH.H₂O (14.71 g, 350.46 mmol) in H₂O (100 mL) at 23° C. and the reaction mixture was stirred at 55° C. for 3 h, Water (200 mL) was added and the aqueous layer was extracted with ethyl acetate (100 mL×2). The aqueous layer was acidified to pH˜1 with a 1M aqueous solution of HCl at 0° C., a quantity of white precipitate was formed, and then the solid was filtered and lyophilized. Compound 1J (18 g, yield: 94.6%) was obtained as a white solid, which was used without further purification. ¹H NMR (400 MHz, DMSO-d₆) δ 13.16-11.67 (m, 1H), 6.68 (br dd, J=7.2, 15.2 Hz, 1H), 5.78 (br d, J=15.6 Hz, 1H), 4.57-4.36 (m, 1H), 4.06 (dd, J=6.5, 9.3 Hz, 1H), 3.78 (br d, J=9.3 Hz, 1H), 1.51 (s, 3H), 1.47-1.31 (m, 12H). MS (ESI) m/z (M+H)⁺ 435.0.

To a solution of compound 1J (18 g, 66.35 mmol) in THF (200 mL) was added Et₃N (20.36 g, 201.17 mmol, 28 mL) and thiophenol (10.96 g, 99.52 mmol, 10.15 mL) at 23° C. and the reaction mixture was stirred at 23° C. for 18 h. 1M aqueous solution of NaOH (100 mL) was added at 0° C. and the aqueous layer was extracted with ethyl acetate (100 mL×2). The aqueous layer was acidified to pH˜1 with a 1M aqueous solution of HCl at 0° C. and extracted with ethyl acetate (250 mL×2). The combined organic phase was dried over Na₂SO₄, filtered and concentrated. Compound 1K (27 g, crude) was obtained as a yellow oil, which was used without further purification.

To a solution of compound 1K (13.5 g, 35.39 mmol) and allyl (2S)-2-amino-3-methyl-butanoate (10.56 g, 38.93 mmol, TFA) in DMF (300 mL) was added HATU (16.15 g, 42.47 mmol), and DIEA (22.63 g, 175.11 mmol, 30.50 mL) at 0° C. and the reaction mixture was stirred at 20° C. for 3 h. Water (700 mL) was added and the aqueous layer was extracted with ethyl acetate (250 mL×3). The combined organic phase was washed with brine (500 mL), dried over Na₂SO₄ and concentrated. The residue was purified by flash silica gel chromatography (ISCO®; 330 g SepaFlash® Silica Flash Column, Eluent of 20% Ethyl acetate/Petroleum ether gradient @ 100 mL/min). Compound 1M (27 g, yield: 73.2%, major diastereomer) was obtained as a white solid, (two batches), ¹H NMR (400 MHz, Acetonitrile-d₃) δ 7.49 (br d, J=7.3 Hz, 2H), 7.36-7.23 (m, 3H), 6.81 (br s, 1H), 6.03-5.86 (m, 1H), 5.40-5.30 (m, 1H), 5.23 (dd, J=1.3, 10.4 Hz, 1H), 4.65-4.56 (m, 2H), 4.45-4.35 (m, 1H), 4.22-4.01 (m, 4H), 2.71-2.58 (m, 1H), 2.43-2.32 (m, 1H), 2.16-2.09 (m, 1H), 1.56 (s, 3H), 1.50-1.29 (m, 12H), 1.00-0.90 (m, 6H). MS (ESI) m/z (M+H)⁺ 521.3. Minor diastereomer (3.6 g, yield: 9.76%) was obtained as a colorless oil. NMR (400 MHz, Acetonitrile-d₃) δ 7.44 (br s, 2H), 7.36-7.20 (m, 3H), 6.84 (br s, 1H), 6.06-5.82 (m, 1H), 5.43 5.25 (m, 1H), 5.19 (dd, J=1.3, 10.6 Hz, 1H), 4.57 (td, J=1.3, 5.5 Hz, 2H), 4.33 (dd, J=6.2, 8.2 Hz, 1H), 4.11-3.87 (m, 4H), 2.48 (br s, 2H), 2.13-2.03 (m, 1H), 1.54 (s, 3H), 1.46-1.28 (m, 12H), 0.92 (d, J=6.8 Hz, 6H), MS (ESI) m/z (M+H)⁺ 521.3.

To a solution of compound 1M (25 g, 48.01 mmol) in CH₃CN (300 mL) was added BiCl₃ (18.17 g, 57.62 mmol) at 20° C. and the reaction mixture was stirred at 20° C. for 4 h. Saturated aqueous NaHCO₃ (400 mL) was added at 0° C., and EA (100 mL) was added, filtered, and the filtrate was extracted with ethyl acetate (200 mL×3), The combined organic phase was washed with brine (500 mL), dried over Na₂SO₄, filtered and concentrated. The residue was purified by flash silica gel chromatography (ISCO®; 120 g SepaFlash® Silica Flash Column, Eluent of 20-40% Ethyl acetate/Petroleum ether gradient @ 85 mL/min). Compound 1N (13 g, yield; 56.3%) was obtained as a colorless oil. ¹H NMR (400 MHz, CDCl₃) δ 7.68-7.52 (m, 1H), 7.48 (d, J=7.7 Hz, 2H), 7.34-7.28 (m, 2H), 7.26-7.21 (m, 1H), 6.02-5.85 (m, 1H), 5.35 (dd, J=1.3, 17.2 Hz, 1H), 5.28-5.18 (m, 1H), 5.10 (br d, J=9.3 Hz, 1H), 4.71-4.58 (m, 2H), 4.50-4.47 (m, 1H), 4.40-4.30 (m, 1H), 3.86-3.62 (m, 3H), 2.75-2.63 (m, 1H), 2.62-2.52 (m, 1H), 2.38-2.20 (m, 2H), 1.47 (s, 9H), 0.98 (t, J=6.8 Hz, 6H). MS (ESI) m/z (M+Boc+H)⁺ 381.2.

To a solution of compound 1N (11.5 g, 23.93 mmol) in THE (300 mL) was added compound 1G (13.62 g, 35.89 mmol), DMAP (300 mg, 2.46 mmol) and EDO (6.88 g, 35.89 mmol) at 0° C. and the reaction mixture was stirred at 20° C. for 16 h, 1M HCl (200 mL) was added and the aqueous layer was extracted with ethyl acetate (200 mL×3). The combined organic phase was washed with saturated aqueous NaHCO₃ (400 mL), dried over Na₂SO₄ and filtered and concentrated. The residue was purified by flash silica gel chromatography (ISCO®; 220 g SepaFlash® Silica Flash Column, Eluent of 30% Ethyl acetate/Petroleum ether gradient @ 100 mL/min). Compound 1P (15 g, yield: 74.4%) was obtained as a white solid. ¹H NMR (400 MHz, Acetonitrile-d₃) δ 7.58-7.50 (m, 2H), 7.41-7.32 (m, 2H), 7.32-7.26 (m, 1H), 7.14 (br s, 1H), 7.08 (d, J=8.5 Hz, 2H), 6.85-6.76 (m, 2H), 6.02-5.82 (m, 3H), 5.62 (br d, J=7.3 Hz, 1H), 5.43-5.31 (m, 1H), 5.30-5.21 (m, 2H), 5.17 (dd, J=1.1, 10.4 Hz, 1H), 4.69-4.57 (m, 2H), 4.48 (br d, J=5.0 Hz, 2H), 4.43-4.25 (m, 3H), 4.23-4.12 (m, 2H), 3.83 (br s, 1H), 3.02 (dd, J=4.9, 14.2 Hz, 1H), 2.78 (dd, J=9.3, 14.1 Hz, 1H), 2.69-2.59 (m, 1H), 2.56-2.43 (m, 1H), 2.19-2.10 (m, 1H), 1.44 (s, 9H), 0.99 (s, 9H), 0.95 (dd, J=4.6, 6.9 Hz, 6H), 0.19 (s, 6H). MS (ESI) m/z (M-Boc+H)⁺ 742.4.

To a solution of compound 1P (14 g, 16.62 mmol) in DCM (150 mL) was added phenylsilane (7.37 g, 68.08 mmol, 8.4 mL) followed by Pd(PPh₃)₄ (1 g, 865.38 umol) at 20° C. and the reaction mixture was stirred at 20° C. for 1 h under N₂. The solvent was then evaporated. The residue was purified by flash silica gel chromatography (ISCO®; 80 g SepaFlash® Silica Flash Column, Eluent of 100% Ethyl acetate 20% MeOH/DCM ether gradient @ 80 mL/min). Compound 1Q (10 g, yield: 72.1%) was obtained as a yellow solid. ¹H NMR (400 MHz, Methanol-d₄) δ 7.57-7.48 (m, 2H), 7.33-7.19 (m, 3H), 7.13-7.00 (m, 2H), 6.89-6.70 (m, 2H), 4.42-4.25 (m, 3H), 4.19 (d, J=5.1 Hz, 1H), 4.09-4.01 (m, 1H), 3.82-3.70 (m, 1H), 3.13-3.00 (m, 1H), 2.97-2.85 (m, 1H), 2.77-2.62 (m, 1H), 2.60-2.47 (m, 1H), 2.29-2.15 (m, 1H), 1.46 (s, 9H), 1.08-0.91 (m, 15H), 0.26-0.13 (m, 6H). MS (ESI) m/z (M+H)⁺ 718.4.

### Tert-butyl ((3S,6S,11R,E)-3-(4-Hydroxybenzyl)-6-isopropyl-2,5,8-trioxo-1-oxa-4,7-Diazacyclododec-9-en-11-yl)carbamate (1)

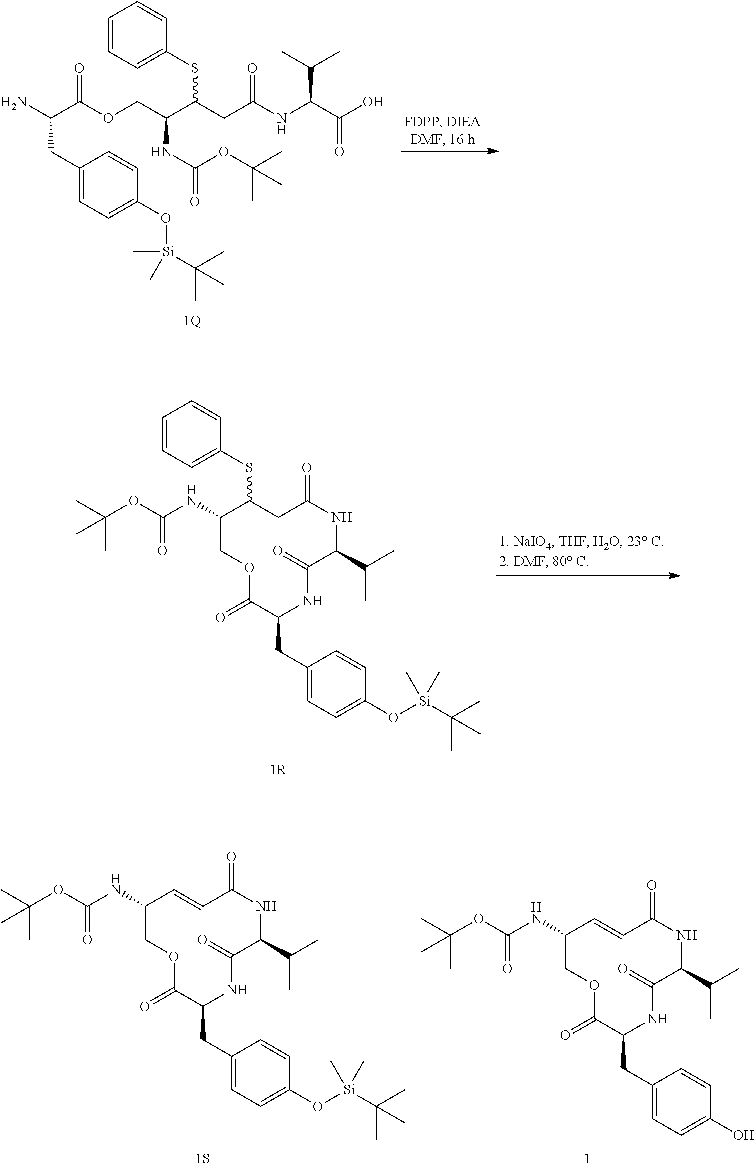 [SMILES: CC(C)[C@H](NC(=O)CC(Sc1ccccc1)[C@H](COC(=O)[C@@H](N)Cc1ccc(O[Si](C)(C)C(C)(C)C)cc1)NC(=O)OC(C)(C)C)C(=O)O ; CC(C)[C@@H]1NC(=O)CC(Sc2ccccc2)[C@@H](NC(=O)OC(C)(C)C)COC(=O)[C@H](Cc2ccc(O[Si](C)(C)C(C)(C)C)cc2)NC1=O ; CC(C)[C@@H]1NC(=O)/C=C/[C@@H](NC(=O)OC(C)(C)C)COC(=O)[C@H](Cc2ccc(O[Si](C)(C)C(C)(C)C)cc2)NC1=O ; CC(C)[C@@H]1NC(=O)/C=C/[C@@H](NC(=O)OC(C)(C)C)COC(=O)[C@H](Cc2ccc(O)cc2)NC1=O]

To a solution of compound 1Q (3 g, 4.18 mmol) in DMF (600 mL) was added FDPP (3.21 g, 8.36 mmol) and DIEA (2.16 g, 16.71 mmol, 2.91 mL). The mixture was stirred at 20° C. for 8 h. The solution was then added with H₂O (3500 mL) at 0° C. and extracted with EA (800 mL×3). The organic phase was washed with brine (1000 mL×2), dried over Na₂SO₄, filtered, and concentrated. The residue was purified by flash silica gel chromatography (ISCO®; 120 g SepaFlash® Silica Flash Column, Eluent of 10% DCM/EA ethergradient @100 mL/min). The residue was purified by flash silica gel chromatography (ISCO®; 80 g SepaFlash® Silica Flash Column, Eluent of 30% EA/PE ether gradient @ 80 mL/min). Compound 1R (3.6 g, yield: 39.4%) was obtained as a yellow solid. ¹H NMR (400 MHz, CDCl₃) δ 7.32-7.25 (m, 2H), 7.21-7.14 (m, 2H), 7.13-7.09 (m, 1H), 6.83 (d, J=8.4 Hz, 2H), 6.74 (d, J=7.6 Hz, 1H), 6.58 (d, J=8.4 Hz, 2H), 6.50 (br d, J=7.4 Hz, 1H), 5.25 (br d, J=9.4 Hz, 1H), 4.22 (br d, J=9.4 Hz, 1H), 4.02-3.93 (m, 1H), 3.92-3.82 (m, 2H), 3.69 (t, J=9.1 Hz, 1H), 3.62-3.52 (m, 1H), 3.12-3.02 (m, 2H), 2.52-2.44 (m, 2H), 1.98 (br s, 1H), 1.30 (s, 9H), 0.84-0.79 (m, 15H), 0.02 (s, 611). MS (ESI) m/z (M-Boc+H)⁺ 600.2.

To a solution of compound 1R (3.5 g, 5.00 mmol) in THF (100 mL) and H₂O (25 mL) was added NaIO₄ (10.69 g, 50.00 mmol) at 20° C. and the reaction mixture was stirred at 15° C. for 24 h, Saturated aqueous NaHCO₃ (250 mL) and EA (200 mL) was added and filtered, the filtrate was extracted with ethyl acetate (100 mL×2). The combined organic phase was washed with brine (300 mL), dried over Na₂SO₄, filtered and concentrated to give a residue.

The crude residue was dissolved in DMF (50 mL) and the solution was heated at 80° C. for 6 h. Saturated aqueous NaHCO₃ (200 mL) was added and the aqueous layer was extracted with ethyl acetate (60 mL×2). The combined organic phase was washed with brine (150 mL), dried over Na₂SO₄, filtered and concentrated. The residue was purified by flash silica gel chromatography (ISCO®; 40 g SepaFlash® Silica Flash Column, Eluent of 0-40% DCM/Ethyl acetate ether gradient @40 mL/min). The residue was purified by flash silica gel chromatography (ISCO®; 40 g SepaFlash®) Silica Flash Column, Eluent of 0-30% Ethyl acetate/Petroleum ether gradient @ 40 mL/min). Compound 1 (350 mg, yield: 14.6%) was obtained as a white solid, ¹H NMR (400 MHz, Methanol-d₄) δ 7.03 (d, J=8.5 Hz, 2H), 6.70 (d, J=8.3 Hz, 2H), 6.49 (dd, J=4.1, 15.7 Hz, 1H), 6.29 (br d, J=15.8 Hz, 1H), 4.71 (br d, J=10.5 Hz, 1H), 4.64-4.36 (m, 2H), 3.84 (br d, J=5.8 Hz, 2H), 3.20-2.97 (m, 2H), 1.94-1.78 (m, 1H), 1.46 (s, 9H), 0.96-0.87 (m, 3H), 0.80 (br d, J=6.8 Hz, 3H). MS (ESI) m/z (M+Na)⁺ 498.0. [α]_{D}²¹ −63.8 (c 1.0, MeOH).

Compound 1S (1.2 g, yield: 40.3%) was obtained as a white solid. ¹H NMR (400 MHz, Methanol-d₄) δ 7.10 (d, J=8.3 Hz, 2H), 6.75 (d, J=8.5 Hz, 2H), 6.59-6.42 (m, 1H), 6.30 (br d, J=15.8 Hz, 1H), 4.71 (br d, J=10.8 Hz, 1H), 4.65-4.43 (m, 2H), 3.85 (br d, J=6.0 Hz, 2H), 3.26-3.04 (m, 2H), 1.93-1.76 (m, 1H), 1.49-1.44 (m, 9H), 1.02-0.99 (m, 9H), 0.90 (d, J=7.0 Hz, 3H), 0.78 (d, J=7.0 Hz, 3H), 0.21-0.16 (m, 6H). MS (ESI) m/z (M+Na)⁺ 612.2. [α]_{D}²¹ −62.2 (c 1.0, MeOH).

### (3S,6S,11R,E)-11-amino-3-(4-((tert-butyldimethylsilyl)oxy)benzyl)-6-isopropyl-1-oxa-4,7-diazacyclododec-9-ene-2,5,8-trione (4)

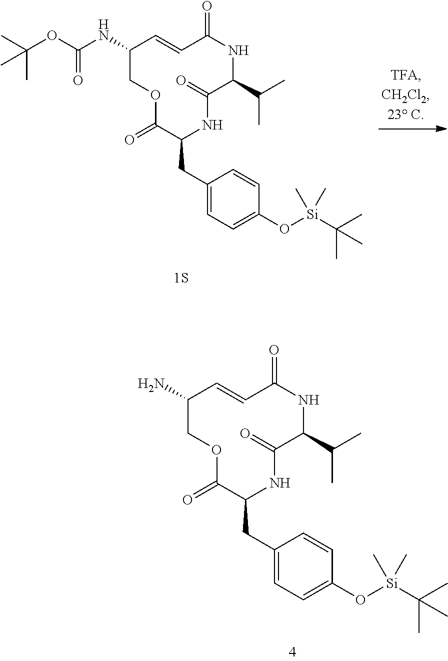 [SMILES: CC(C)[C@@H]1NC(=O)/C=C/[C@@H](NC(=O)OC(C)(C)C)COC(=O)[C@H](Cc2ccc(O[Si](C)(C)C(C)(C)C)cc2)NC1=O ; CC(C)[C@@H]1NC(=O)/C=C/[C@@H](N)COC(=O)[C@H](Cc2ccc(O[Si](C)(C)C(C)(C)C)cc2)NC1=O]

To a solution of compound 1S (200 mg, 339.10 umol) in DCM (5 mL) was added TFA (1 mL). The mixture was stirred at 15° C. for 3 h. Saturated aqueous NaHCO₃ (30 mL) was added and the aqueous layer was extracted with CH₂Cl₂ (20 mL×3). The combined organic phase was dried over Na₂SO₄, filtered and concentrated. The residue was dissolved in EA (0.5 mL) and then added PE (10 mL), a quantity of precipitate was formed, then filtered. Compound 4 (130 mg, yield: 75.6%) was obtained as a white solid. ¹H NMR (400 MHz, Methanol-d₄) δ 7.08 (br d, J=8.3 Hz, 2H), 6.74 (br d, J=8.3 Hz, 2H), 6.53 (dd, J=3.8, 15.8 Hz, 1H), 6.43 6.27 (m, 1H), 4.72-4.58 (m, 1H), 4.55-4.36 (m, 1H), 3.89-3.73 (m, 3H), 3.13 (br d, J=7.3 Hz, 2H), 1.93-1.71 (m, 1H), 0.98 (s, 9H), 0.93-0.88 (m, 3H), 0.77 (d, J=6.8 Hz, 3H), 0.17 (s, 6H). MS (ESI) m/z (M+H)⁺ 490.3. [α]_{D}²¹ −50.7 (c 1.0, MeOH).

### Tert-butyl ((S)-1-(((3S,6S,11R,E)-3-(4-hydroxybenzyl)-6-isopropyl-2,5,8-trioxo-1-oxa-4,7-diazacyclododec-9-en-11-yl)amino)-3-methyl-1-oxobutan-2-yl)carbamate (2)

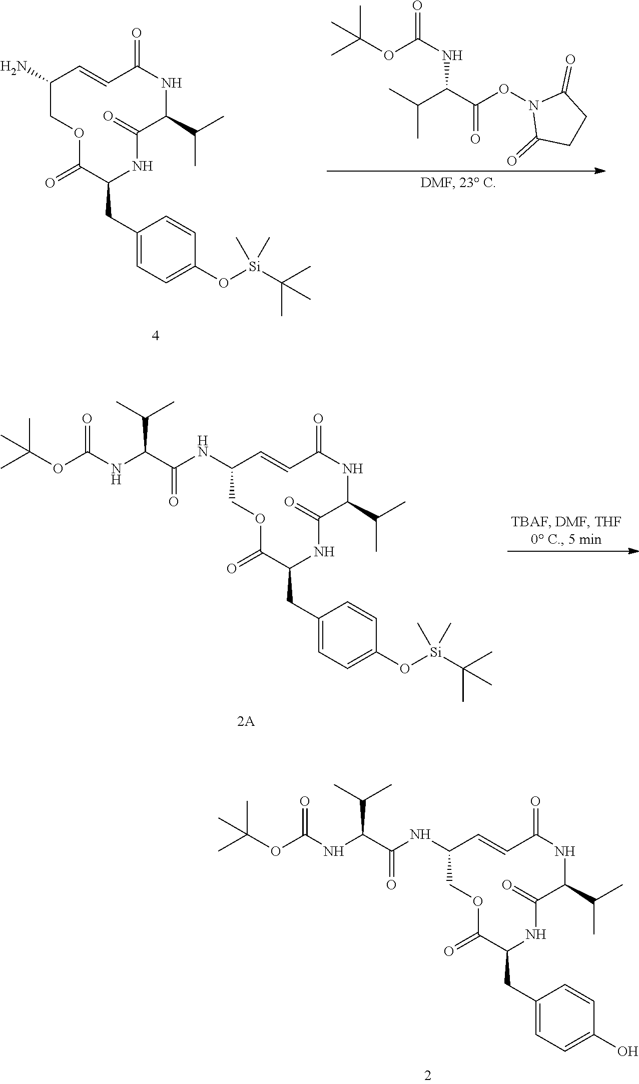

To a solution of compound 4 (90 mg, 183.79 umol) in DMF (5 mL) was added (2,5-dioxopyrrolidin-1-yl) (2S)-2-(tert-butoxycarbonylamino)-3-methyl-butanoate (90 mg, 286.32 umol) at 20° C. and the reaction mixture was stirred at 20° C. for 36 h. The reaction was quenched with saturated NH₄Cl (5 mL), and then H₂O (10 mL) was added and the aqueous layer was extracted with EA (10 mL×3). The organic phase was washed with brine (20 mL). The organic phase was dried over Na₂SO₄, filtered and concentrated. The residue was purified by preparatory-TLC (SiO₂, DCM:EA=1:5). Compound 2A (85 mg, yield: 65.1%) was obtained as a white solid. ¹H NMR (400 MHz, Methanol-d₄) δ 7.09 (d, J=8.4 Hz, 2H), 6.74 (d, J=8.4 Hz, 2H), 6.52 (dd, J=4.2, 15.7 Hz, 1H), 6.23 (br d, J=15.2 Hz, 1H), 4.84-4.53 (m, 1H), 4.00 (br d, J=6.6 Hz, 1H), 3.86 (br d, J=11.0 Hz, 1H), 3.82-3.75 (m, 1H), 3.21-2.98 (m, 2H), 2.15-1.98 (m, 1H), 1.88-1.79 (m, 1H), 1.45 (s, 9H), 1.03-0.86 (m, 18H), 0.76 (br d, J=6.8 Hz, 3H), 0.17 (s, 6H). MS (ESI) m/z (M+Boc+H)⁺ 589.2.

To a solution of compound 2A (70 mg, 101.61 umol) in DMF (3 mL) and THF (3 mL) was added TBAF (1M, 152.41 uL). The mixture was stirred at 0° C. for 15 min. The reaction was diluted with EA (10 mL), quenched with saturated NH₄Cl (5 mL), and diluted with H₂O (20 mL), extracted with EA (10 mL×3). The organic phase was washed with H₂O (30 mL×3) and brine (40 mL×2), dried over Na₂SO₄, filtered, and concentrated. The residue was dissolved in EA (0.5 mL), and then added PE (15 mL), the precipitate was formed, and the solid was filtered and was dried in vacuo. The residue was further separated by SFC (condition: column: DAICEL CHIRALCEL OD-H (250 mm*30 mm, 5 μm); mobile phase: [0.1% NH₃.H₂O EtOH]; B %: 20%-20%, min). Compound 2 (25 mg, yield: 40.3%) was obtained as a white solid. ¹H NMR (400 MHz, CDCl₃) δ 7.20 (br s, 2H), 7.03 (br d, J=8.5 Hz, 2H), 6.78 (d, J=8.3 Hz, 2H), 6.57 (dd, J=3.3, 15.6 Hz, 1H), 6.24 (br s, 1H), 6.12 (br d, J=15.6 Hz, 1H), 5.25 (br s, 1H), 5.01 (br s, 1H), 4.94-4.78 (m, 2H), 4.13 (br s, 1H), 3.79 (br s, 1H), 3.62 (br d, J=10.8 Hz, 1H), 3.02 (br dd, J=7.9, 14.2 Hz, 1H), 2.76 (br dd, J=8.2, 13.9 Hz, 1H), 2.16 (br s, 1H), 2.03-1.96 (m, 1H), 1.45 (s, 9H), 1.01 (d, J=6.8 Hz, 6H), 0.92 (dd, J=6.8, 12.3 Hz, 6H). MS (ESI) m/z (M+Boc+H)⁺ 475.1. [α]_{D}²³ −22.4 (c 1.0, MeOH).

### Example 2

### Compound 3

### Tert-butyl ((S)-1-(((S)-1-(((3S,6S,11R,E)-3-(4-Hydroxybenzyl)-6-isopropyl-2,5,8-trioxo-1-oxa-4,7-diazacyclododec-9-en-11-yl)amino)-3-methyl-1-oxobutan-2-yl)amino)-3-(4-hydroxyphenyl)-1-oxopropan-2-yl)carbamate (3)

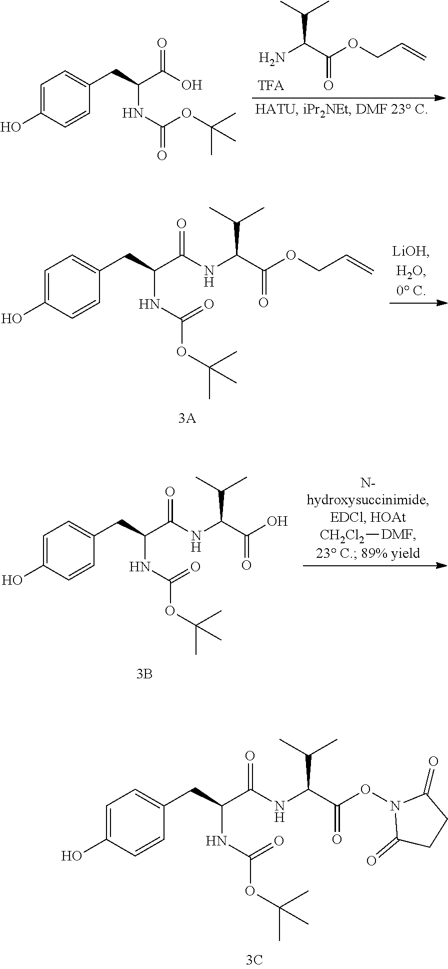

To a solution of (tertbutoxycarbonyl)-L-tyrosine (1 g, 3.55 mmol), allyl (2S)-2-amino-3-methyl-butanoate (synthesized using procedure for compound 2) (1.01 g, 3.73 mmol, TFA) in DMF (20 mL) was added followed by the addition of DIEA (1.48 g, 11.48 mmol, 2.00 mL) and HATU (1.49 g, 3.91 mmol) at 0° C. The reaction was stirred for 2 h at 20° C. The reaction was quenched with water (80 mL) at 0° C., and extracted with ethyl acetate (50 mL×2). The combined organic phase was washed with brine (100 mL), dried over Na₂SO₄, filtered and concentrated. The residue was purified by flash silica gel chromatography (ISCO®; 12 g SepaFlash® Silica Flash Column, Eluent of 40% Ethylacetate/Petroleum ether gradient @ 30 mL/min). Compound 3A (1.48 g, yield: 99.0%) was obtained as a white solid. ¹H NMR (400 MHz, CDCl₃) δ 7.03 (br d, J=8.4 Hz, 2H), 6.73 (d, J=8.4 Hz, 2H), 6.64 (br s, 1H), 6.49 (br d, J=8.6 Hz, 1H), 5.99-5.81 (m, 1H), 5.38-5.22 (m, 2H), 5.12 (br s, 1H), 4.60 (br d, J=5.7 Hz, 2H), 4.48 (br dd, J=5.0, 8.5 Hz, 1H), 4.35-4.25 (m, 1H), 4.30 (br d, J=5.3 Hz, 1H), 2.98 (br d, J=6.4 Hz, 2H), 2.18-2.07 (m, 1H), 1.42 (s, 9H), 0.88 (dd, J=6.8, 12.1 Hz, 6H). MS (ESI) m/z (M+Boc+H)⁺ 321.2.

To a solution of compound 3A (1.4 g, 3.33 mmol) in THF (15 mL) was added LiOH.H₂O (420 mg, 10.01 mmol) in H₂O (5 mL) at 0° C. The mixture was stirred at 0° C. for 1.5 hr. The reaction mixture was diluted with water (20 mL), extracted with EA (20 mL×2), then the water layers were acidified with 1N HCl to pH 1-2, extracted with EA (30 mL×2), the organic layers were dried over Na₂SO₄, filtered and concentrated to give a residue. Compound 3B (1 g, yield: 78.9%) was obtained as a white solid, which was used into the next step without further purification. ¹H NMR (400 MHz, DMSO-d₆) δ 12.62 (br s, 1H), 9.16 (s, 1H), 7.84 (br d, J=8.6 Hz, 1H), 7.05 (br d, J=8.2 Hz, 2H), 6.88 (d, J=8.8 Hz, 1H), 6.64 (br d, J=8.2 Hz, 2H), 4.23-4.08 (m, 2H), 2.84 (dd, J=3.7, 13.7 Hz, 1H), 2.61 (dd, J=11.0, 13.7 Hz, 1H), 2.14-2.00 (m, 1H), 1.31 (s, 9H), 0.89 (dd, J=2.7, 6.7 Hz, 6H). MS (ESI) m/z (M+Boc+H)⁺ 281.1.

To a solution of compound 3B (1 g, 2.63 mmol) and N-hydroxysuccinimide (605 mg, 5.26 mmol) in DCM (15 mL) and DMF (3 mL) was added EDCI (756 mg, 3.94 mmol) and HOAt (537 mg, 3.95 mmol) at 0° C. for 1 h. The mixture was stirred at 20° C. for 15 h. Saturated NH₄Cl (50 mL) was added and the aqueous layer was extracted with DCM (30 mL×2). The organic phase was washed with brine (50 mL), dried over Na₂SO₄, filtered and concentrated. The residue was purified by flash silica gel chromatography (ISCO®; 24 g SepaFlash® Silica Flash Column, Eluent of 50% Ethylacetate/Petroleum ether gradient @ 30 mL/min). Compound 3C (0.7 g, yield: 48.0%) was obtained as a white solid. ¹H NMR (400 MHz, CDCl₃) δ 7.04 (d, J=8.5 Hz, 2H), 6.84-6.69 (m, 2H), 6.43 (d, J=8.5 Hz, 1H), 5.99 (br s, 1H), 5.19 (br s, 1H), 4.82 (br dd, J=4.6, 8.4 Hz, 1H), 4.27 (br d, J=7.3 Hz, 1H), 3.07-2.92 (m, 2H), 2.85 (s, 4H), 2.38-2.20 (m, 1H), 1.43 (s, 9H), 0.97 (t, J=7.2 Hz, 6H). MS (ESI) m/z (M+Boc+H)⁺ 378.1.

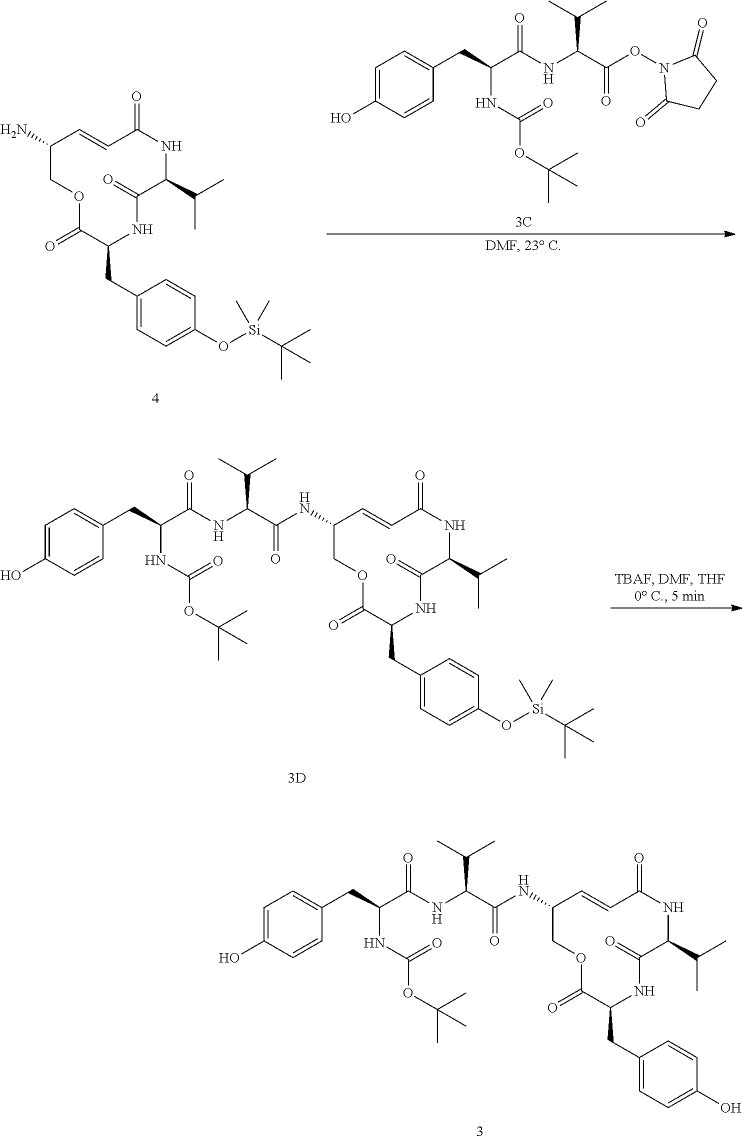 [SMILES: CC(C)[C@@H]1NC(=O)/C=C/[C@@H](N)COC(=O)[C@H](Cc2ccc(O[Si](C)(C)C(C)(C)C)cc2)NC1=O ; CC(C)[C@H](NC(=O)[C@H](Cc1ccc(O)cc1)NC(=O)OC(C)(C)C)C(=O)ON1C(=O)CCC1=O ; CC(C)[C@@H]1NC(=O)/C=C/[C@@H](NC(=O)[C@@H](NC(=O)[C@H](Cc2ccc(O)cc2)NC(=O)OC(C)(C)C)C(C)C)COC(=O)[C@H](Cc2ccc(O[Si](C)(C)C(C)(C)C)cc2)NC1=O ; CC(C)[C@@H]1NC(=O)/C=C/[C@@H](NC(=O)[C@@H](NC(=O)[C@H](Cc2ccc(O)cc2)NC(=O)OC(C)(C)C)C(C)C)COC(=O)[C@H](Cc2ccc(O)cc2)NC1=O]

To a solution of compound 4 (100 mg, 204.22 umol) in DMF (5 mL) was added compound 3C (200 mg, 418.84 umol) at 20° C. and the reaction mixture was stirred at 20° C. for 16 h. The reaction was quenched with saturated NH₄Cl (5 mL), and then H₂O (10 mL) was added and the aqueous layer was extracted with EA (10 mL×3). The organic phase was washed with brine (20 mL), dried over Na₂SO₄, filtered and concentrated. The residue was purified by preparatory-TLC (SiO₂, EA:DCM=5:1). Compound 3D (120 mg, yield: 68.3%) was obtained as a white solid. ¹H NMR (400 MHz, Methanol-d₄) δ 7.09-7.04 (m, 4H), 6.77-6.67 (m, 4H), 6.58-6.46 (m, 1H), 6.32-6.17 (m, 1H), 4.83-4.70 (m, 2H), 4.67-4.55 (m, 1H), 4.35-4.22 (m, 2H), 3.89-3.75 (m, 2H), 3.20-3.00 (m, 3H), 2.80-2.75 (m, 1H), 2.21-2.02 (m, 1H), 1.93-1.77 (m, 1H), 1.41-1.34 (m, 9H), 1.03-0.85 (m, 18H), 0.81-0.71 (m, 3H), 0.17 (s, 6H). MS (ESI) m/z (M+Boc+H)⁺ 752.2.

To a solution of compound 3D (100 mg, 117.36 umol) in DMF (3 mL) and THF (3 mL) was added TBAF (1M, 176.04 uL). The mixture was stirred at 0° C. for 15 min. The reaction was diluted with EA (10 mL), quenched with saturated NH₄Cl (5 mL), and diluted with H₂O (20 mL), extracted with EA (10 mL×3). The organic phase was washed with H₂O (30 mL×3) and brine (40 mL×2), dried over Na₂SO₄, filtered, and concentrated. The residue was dissolved in EA (1 mL), and then added PE (15 mL), the precipitate was formed, and the solid was filtered and was dried in vacuo. The residue was obtained as a white solid, which was further separated by SFC (condition: column: DAICEL CHIRALCEL OD-H (250 mm*30 mm, Sum); mobile phase: [0.1% NH₃H₂O EtOH]; B %; 30%-30%, min). Compound 3 (45 mg, yield: 51.5%) was obtained as a white solid. ¹H NMR (400 MHz, Methanol-d₄) δ 7.06-6.97 (m, 4H), 6.67 (br d, J=8.3 Hz, 4H), 6.54-6.42 (m, 1H), 6.26-6.16 (m, 1H), 4.81-4.68 (m, 2H), 4.57 (br s, 1H), 4.31-4.18 (m, 2H), 3.86-3.73 (m, 2H), 3.17 3.07 (m, 1H), 3.05-2.90 (m, 21:1), 2.83-2.69 (m, 1H), 2.13-2.04 (m, III), 1.90-1.78 (m, 1H), 1.41-1.32 (m, 9H), 0.98-0.90 (m, 6H), 0.87 (d, J=6.8 Hz, 3H), 0.79-0.73 (m, 3H). MS (ESI) m/z (M+Boc+H)⁺ 638.1. [α]_{D}²³ −38.3 (c 1.0, MeOH).

### Example 3

### Compound 5

### Tert-butyl ((3S,6S,11R,E)-3-benzyl-6-isopropyl-2,5,8-trioxo-1-oxa-4,7-diazacyclododec-9-en-11-yl)carbamate (5)

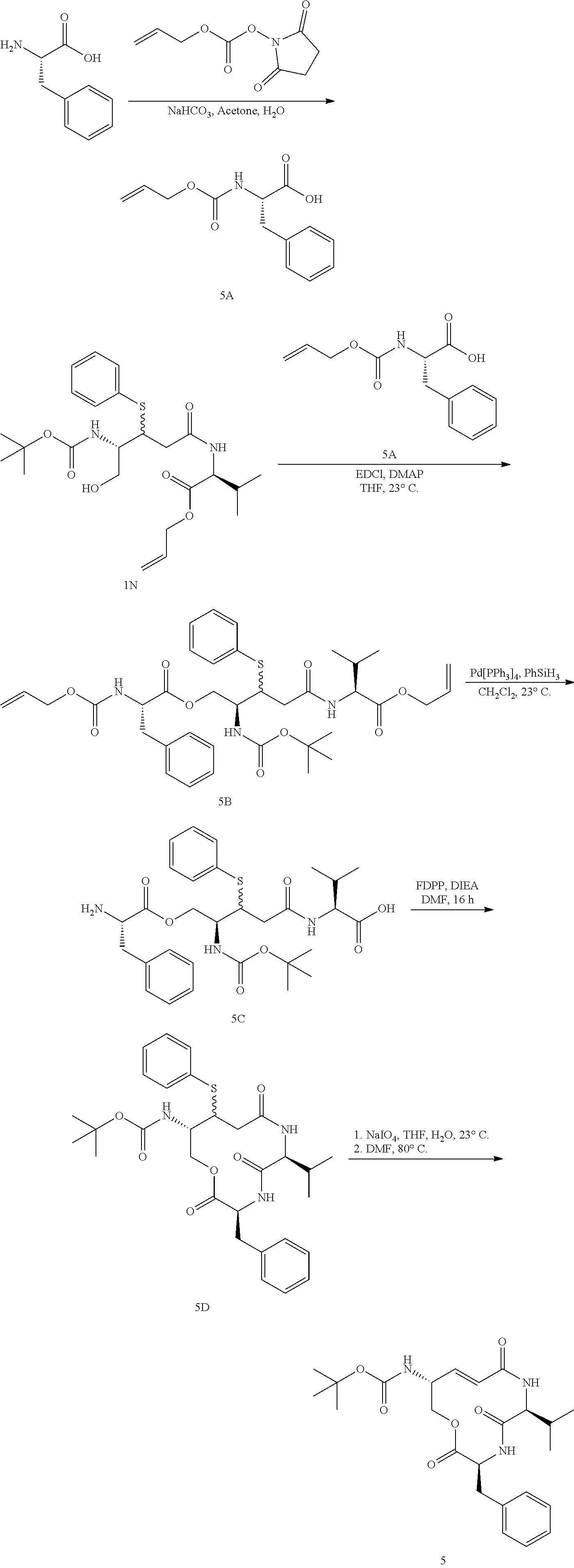

To a solution of L-phenylalanine (5 g, 30.27 mmol) and NaHCO₃ (7.63 g, 90.81 mmol) in acetone (70 mL) and H₂O (70 mL) was added allyl (2,5-dioxopyrrolidin-1-yl) carbonate (6.63 g, 33.30 mmol). The mixture was stirred at 15° C. for 16 h. The reaction was filtered, and the filtrate was extracted with EA (50 mL×2), the water phase was adjusted with HCl (1N) to pH˜3, and then extracted with EA (50 mL×3). The organic phase was dried over Na₂SO₄, filtered, and concentrated. Compound 5A (6 g, yield: 78.7%) was obtained as a colorless oil, which was used to the next step without purification. ¹H NMR (400 MHz, DMSO-d₆) δ 12.73 (br s, 1H), 7.60 (d, J=8.6 Hz, 1H), 7.37-7.15 (m, 5H), 5.99-5.75 (m, 1H), 5.36-5.01 (m, 2H), 4.48-4.32 (m, 2H), 4.17-4.11 (m, 1H), 3.06 (dd, J=4.4, 13.9 Hz, 1H), 2.82 (dd, J=10.8, 13.7 Hz, 1H). MS (ESI) m/z (M+H)⁺ 250.1.

Intermediates 5A and 1N were subjected to synthetic procedures as for compound 1S to yield the compound 5. Compound 5 (40 mg, yield: 33.0%) as a white solid was obtained. ¹H NMR (400 MHz, Methanol-d₄) δ 7.31-7.18 (m, 5H), 6.56-6.44 (m, 1H), 6.37-6.20 (m, 1H), 4.76-4.48 (m, 3H), 3.90-3.80 (m, 2H), 3.30-3.15 (m, 2H), 1.89-1.75 (m, 1H), 1.46 (s, 9H), 0.88 (d, J=7.0 Hz, 3H), 0.75 (d, J=6.8 Hz, 3H). MS (ESI) m/z (M+Na)+482.0.

### Example 4

### Compound 8 tert-butyl ((s)-1-((2-(((3S,6S,11R,E)-3-(4-hydroxybenzyl)-6-isopropyl-2,5,8-trioxo-1-oxa-4,7-diazacyclododec-9-en-11-yl)amino)-2-oxoethyl)amino)-3-(4-hydroxyphenyl)-1-oxopropan-2-yl)carbamate (8)

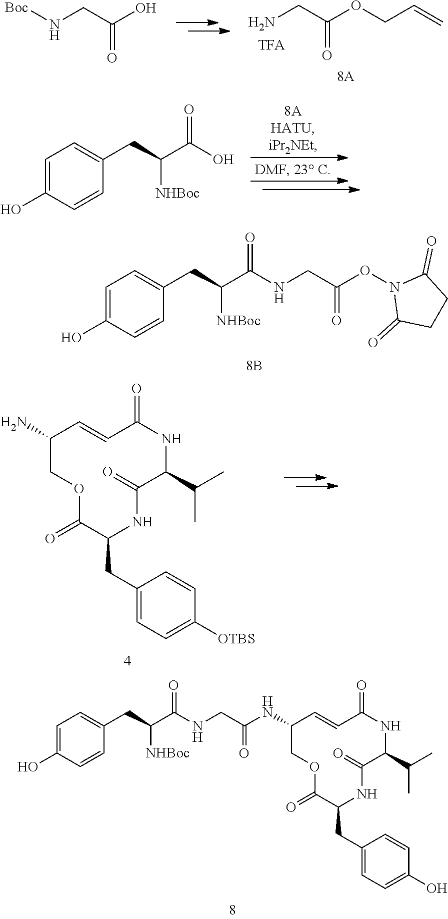

A solution of (tert-butoxycarbonyl)glycine was subjected to same conditions intermediate 1C to obtain the intermediate 8A. Compound 8A (8.3 g, yield: 97.5%, TFA) was obtained as light yellow oil. ¹H NMR (400 MHz, CDCl₃) δ 9.43 (s, 1H), 8.16 (s, 2H), 5.91-5.81 (m, 1H), 5.39-5.24 (m, 2H), 4.67 (d, J=6.1 Hz, 2H), 3.88 (s, 2H).

Intermediates 8A and (tert-butoxycarbonyl)-L-tyrosine were subjected to same conditions as 3C to yield the intermediate 8B. Compound 8B (940 mg, yield: 39.3%) as colorless gel was obtained. ¹H NMR (400 MHz, DMSO-d₆) δ 9.22-9.06 (m, 1H), 8.61 (t, J=5.9 Hz, 1H), 7.12-7.00 (m, 2H), 6.92 (d, J=8.8 Hz, 1H), 6.63 (d, J=8.3 Hz, 2H), 4.43-4.20 (m, 2H), 4.16-4.07 (m, 1H), 2.95-2.83 (m, 2H), 2.81 (s, 4H), 2.66-2.58 (m, 1H), 1.29 (s, 9H).

Intermediate 8B and compound 4 were subjected to synthetic procedures as for compound 3 to yield the compound 8. Compound 8 (32 mg, yield: 91.8%) as a white solid was obtained. ¹H NMR (400 MHz, DMSO-d₆) δ 9.12 (br s, 2H), 8.38 (br s, 1H), 8.21 (br s, 1H), 7.53 (br s, 1H), 7.03-6.84 (m, 6H), 6.61-6.57 (m, 4H), 6.31-6.18 (m, 2H), 4.73 (br s, 1H), 4.50 (br d, J=9.5 Hz, 1H), 4.23 (br s, 1H), 4.10 (br s, 1H), 3.80-3.65 (m, 4H), 2.99-2.88 (m, 3H), 2.72-2.62 (m, 1H), 1.83-1.68 (m, 1H), 1.23 (s, 9f), 0.83-0.78 (m, 3H), 0.67 (br d, J=6.8 Hz, 3H). MS (ESI) m/z (M−Boc+H)⁺ 596.0.

### Example 5

### Compound 7

### tert-butyl (2-(((s)-1-(((3S,6S,11R,E)-3-(4-hydroxybenzyl)-6-isopropyl-2,5,8-trioxo-1-oxa-4,7-diazacyclododec-9-en-11-yl)amino)-3-methyl-1-oxobutan-2-yl)amino)-2-oxoethyl)carbamate (7)

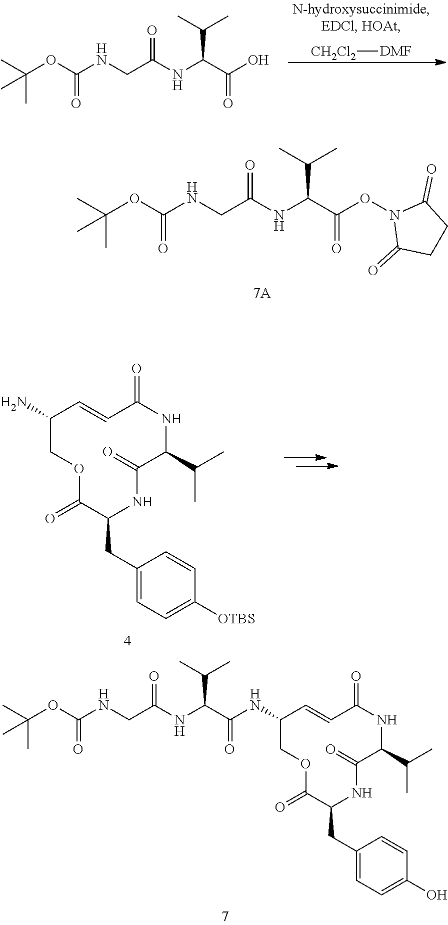

To a solution of (tert-butoxycarbonyl)glycyl-L-valine (500 mg, 1.82 mmol) and N-hydroxysuccinimide (420 mg, 3.65 mmol) in DMF (3 mL) and DCM (15 mL) was added EDCI (525 mg, 2.74 mmol) and HOAt (372 mg, 2.73 mmol). The mixture was stirred at 20° C. for 16 h. Saturated NH₄Cl (50 mL) was added and the aqueous layer was extracted with DCM (30 mL×2). The organic phase was washed with brine (50 mL), dried over Na₂SO₄, filtered and concentrated. The residue was triturated in EA(5 mL):PE(1 mL), filtered and dried in vacuo. Compound 7A (400 mg, yield: 59.1%) was obtained as a white solid. ¹H NMR (400 MHz, DMSO-d₆) δ 8.35 (d, J=8.3 Hz, 1H), 6.98 (t, J=6.1 Hz, 1H), 4.62 (dd, J=6.0, 8.2 Hz, 1H), 3.77-3.48 (m, 2H), 2.82 (br s, 4H), 2.23-2.15 (m, 1H), 1.38 (s, 9H), 0.99 (dd, J=3.2, 6.6 Hz, 7H). MS (ESI) m/z (M+Na)⁺ 394.1.

Intermediate 7A and compound 4 were subjected to synthetic procedures as for compound 3 to yield the compound 7. Compound 7 (90 mg, yield: 82.9%) as a white solid was obtained. ¹H NMR (400 MHz, Methanol-d₄) 7.05 (d, J=8.3 Hz, 2H), 6.69 (d, J=8.3 Hz, 2H), 6.48 (dd, J=3.6, 15.7 Hz, 1H), 6.22 (br d, J=15.6 Hz, 1H), 4.88-4.55 (m, 3H), 4.26 (d, J=6.0 Hz, 1H), 3.96-3.78 (m, 4H), 3.17-3.05 (m, 1H), 3.03-2.85 (m, 1H), 2.27-2.11 (m, 1H), 1.94-1.85 (m, 1H), 1.46 (s, 9H), 1.01 (d, J=6.8 Hz, 3H), 0.97 (d, J=6.8 Hz, 3H), 0.95-0.90 (DMFm, 3H), 0.82 (d, J=6.8 Hz, 3H). MS (ESI) m/z (M−Boc+H)⁺ 532.2.

### Example 6

### Compound 6

### tert-butyl ((s)-1-((s)-1-(((3S,6S,11R,E)-3-(4-hydroxybenzyl)-6-isopropyl-2,5,8-trioxo-1-oxa-4,7-diazacyclododec-9-en-11-yl)amino)-3-methyl-oxobutan-2-yl)amino)-1-oxo-3-phenylpropan-2-yl)carbamate (6)

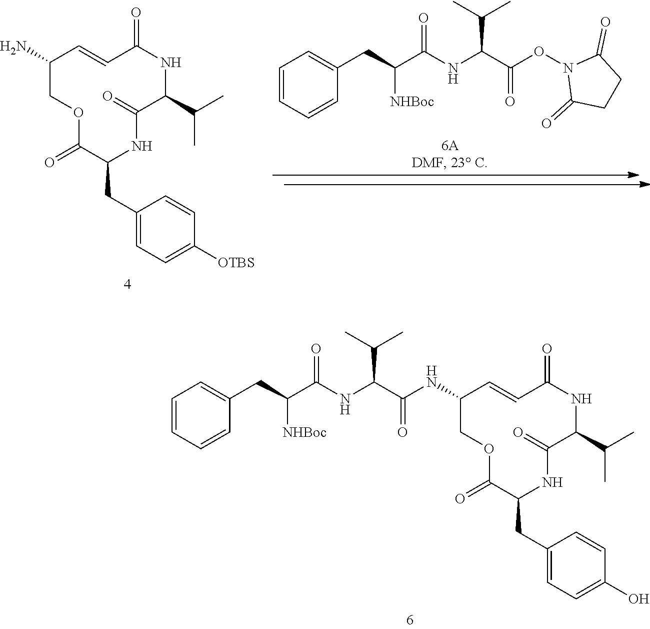 [SMILES: CC(C)[C@@H]1NC(=O)/C=C/[C@@H](N)COC(=O)[C@H](Cc2ccc(O[Si](C)(C)C(C)(C)C)cc2)NC1=O ; CC(C)[C@H](NC(=O)[C@H](Cc1ccccc1)NC(=O)OC(C)(C)C)C(=O)ON1C(=O)CCC1=O ; CC(C)[C@@H]1NC(=O)/C=C/[C@@H](NC(=O)[C@@H](NC(=O)[C@H](Cc2ccccc2)NC(=O)OC(C)(C)C)C(C)C)COC(=O)[C@H](Cc2ccc(O)cc2)NC1=O]

Intermediate 6A was synthesized from (tert-butoxycarbonyl)-L-phenylalanine using same conditions intermediate 3C to obtain the intermediate 6A. Compound 6A (0.8 g, yield: 63.2%) was obtained as a white solid. ¹H NMR (400 MHz, CDCl₃) δ 7.32-7.25 (m, 2H), 7.24-7.17 (m, 3H), 6.42 (br d, J=8.3 Hz, 1H), 5.04 (br s, 1H), 4.84 (dd, J=4.8, 8.7 Hz, 1H), 4.39-4.29 (m, 1H), 3.07 (d, J=6.8 Hz, 2H), 2.83 (br s, 4H), 2.36-2.16 (m, 1H), 1.76-1.50 (m, 1H), 1.39 (s, 9H), 0.96 (t, J=7.0 Hz, 6H). MS (ESI) m/z (M+Na)⁺ 484.2.

Intermediate 6A and compound 4 were subjected to synthetic procedures as for compound 3 to yield the compound 6. Compound 6 (70 mg, yield: 65.5%) as a white solid was obtained. ¹H NMR (400 MHz, Methanol-d₄) δ 7.31-7.25 (m, 4H), 7.22 (br d, J=6.0 Hz, 1H), 7.04 (d, J=8.3 Hz, 2H), 6.70 (d, J=8.3 Hz, 2H), 6.52 (dd, J=3.9, 15.4 Hz, 1H), 6.24 (br d, J=15.6 Hz, 1H), 4.86-4.71 (m, 2H), 4.68-4.55 (m, 1H), 4.41-4.37 (m, 1H), 4.32-4.23 (m, 1H), 3.88-3.76 (m, 2H), 3.22-3.09 (m, 2H), 3.06-2.96 (m, 1H), 2.89-2.84 (m, 1H), 2.21-2.08 (m, 1H), 1.93-1.84 (m, 1H), 1.92-1.81 (m, 1H), 1.38 (s, 9H), 0.98 (br t, J=5.9 Hz, 6H), 0.91 (br d, J=6.8 Hz, 3H), 0.79 (br d, J=6.8 Hz, 3H). MS (ESI) m/z (M+Na)⁺ 744.3.

### Example 7

### Compound 9

### Benzyl ((s)-1-(((3S,6S,11R,E)-3-(4-hydroxybenzyl)-6-isopropyl-2,5,8-trioxo-1-oxa-4,7-diazacyclododec-9-en-11-yl)amino)-3-methyl-1-oxobutan-2-yl)carbamate (9)

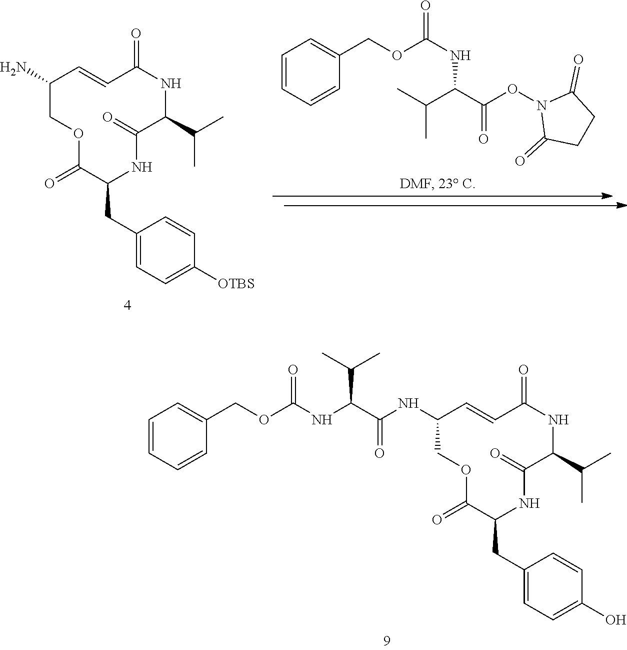 [SMILES: CC(C)[C@@H]1NC(=O)/C=C/[C@@H](N)COC(=O)[C@H](Cc2ccc(O[Si](C)(C)C(C)(C)C)cc2)NC1=O ; CC(C)[C@H](NC(=O)OCc1ccccc1)C(=O)ON1C(=O)CCC1=O ; CC(C)[C@@H]1NC(=O)/C=C/[C@@H](NC(=O)[C@@H](NC(=O)OCc2ccccc2)C(C)C)COC(=O)[C@H](Cc2ccc(O)cc2)NC1=O]

Intermediate 2,5-dioxopyrrolidin-1-yl ((benzyloxy)carbonyl)-L-valinate and compound 4 were subjected to synthetic procedures as for compound 3 to yield the compound 9. Compound 9 (50 mg, yield: 71.2%) as a white solid was obtained. ¹H NMR (400 MHz, Methanol-d₄) δ 7.44-7.23 (m, 5H), 7.03 (d, J=8.3 Hz, 2H), 6.68 (d, J=8.3 Hz, 2H), 6.50 (dd, J=3.8, 15.8 Hz, 1H), 6.23 (d, J=15.2 Hz, 1H), 5.13 (s, 2H), 4.87-4.51 (m, 3H), 4.04 (d, J=6.8 Hz, 1H), 3.90-3.70 (m, 2H), 3.17-3.05 (m, 1H), 3.02-2.89 (m, 1H), 2.20-2.06 (m, 1H), 1.88-1.79 (m, 1H), 1.04-0.85 (m, 9H), 0.76 (d, J=6.8 Hz, 3H). MS (ESI) m/z (M+H)⁺ 609.1.

### Example 8

### Compound 11

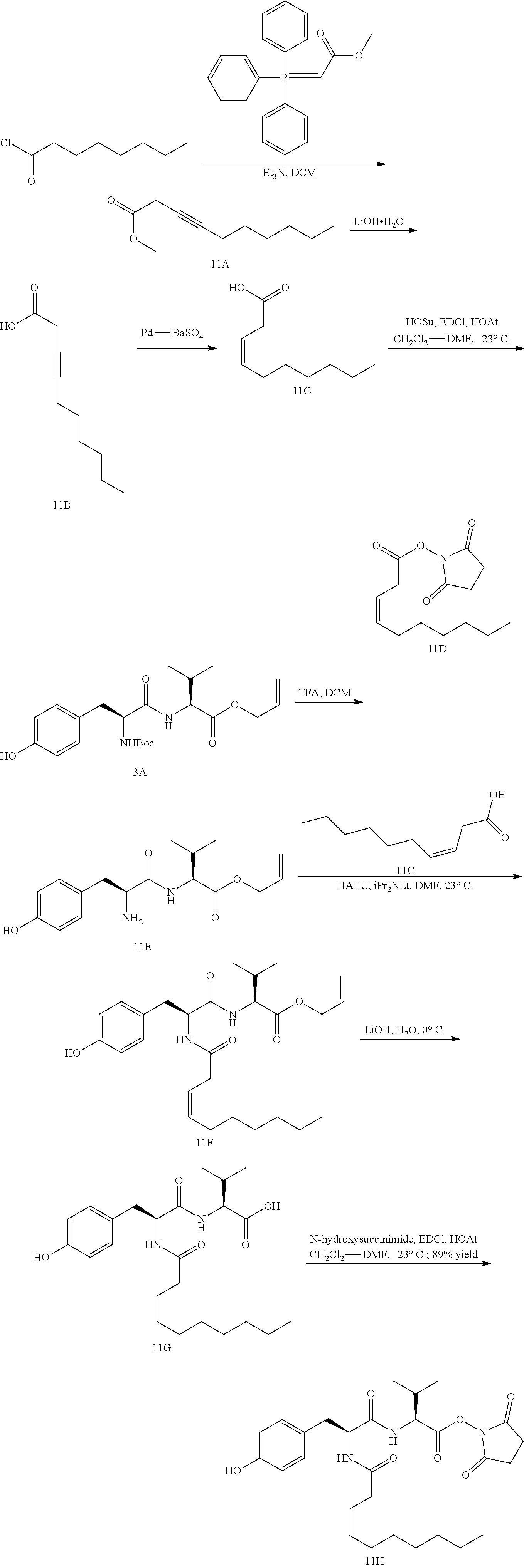

Methyl 2-(triphenyl-phosphanylidene)acetate (12.33 g, 36.89 mmol) was stirred with Et₃N (3.73 g, 36.89 mmol, 5.13 mL) in dry DCM (110 mL) with ice-bath cooling at 0° C. Octanoyl chloride (6 g, 36.89 mmol, 6.3 mL) was added dropwise and the mixture was allowed to warm to 15° C. then stirred for 16 h. Approximately half of the solvent was removed in vacuo and the residue was passed through a short plug of silica eluting with dichloromethane. The solvent was removed in vacuo. The residue was purified by flash silica gel chromatography (ISCO®; 40 g SepaFlash®) Silica Flash Column, Eluent of 10% Ethyl acetate/Petroleum ether gradient @ 40 mL/min). Compound 11A (5 g, yield: 74.4%) was obtained as a yellow oil. ¹H NMR (400 MHz, CDCl₃) δ 5.67-5.53 (m, 2H), 3.73 (s, 3H), 2.18-2.06 (m, 2H), 1.51-1.40 (m, 2H), 1.37-1.19 (m, 6H), 0.88 (t, J=6.8 Hz, 3H).

To a solution of compound 1A (2 g, 10.97 mmol) in THF (10 mL) was added LiOH.H₂O (2.3 g, 54.87 mol) in H₂O (10 mL). The mixture was stirred at 20° C. for 1 h. The mixture was then diluted with MTBE (60 mL) and water (60 mL) and the layers were separated. The aqueous layer was acidified with 1M aqueous hydrochloric acid to pH <1 and extracted with MTBE (50 mL×2). The combined organic layers were dried over sodium sulfate and concentrated in vacuo. The residue was used to the next step without purification. Compound 11B (1.6 g, yield: 86.7%) was obtained as a colorless oil. ¹H NMR (400 MHz, CDCl₃) δ 11.31 (br s, 1H), 3.39-3.20 (m, 2H), 2.27-2.04 (m, 2H), 1.55-1.41 (m, 2H), 1.39-1.15 (m, 6H), 0.86 (t, J=6.8 Hz, 3H).

To a solution of compound 11B (1.3 g, 7.73 mmol) in THF (30 mL) was added Lindlar catalyst (0.2 g, 968.48 umol). The suspension was degassed under vacuum and purged with H₂ several times. The mixture was stirred under H₂ balloon at 15° C. for 30 min. Filtered, and the filtrate was concentrated. The residue was used to the next step without purification. Compound 11C (1.3 g, yield: 98.8%) was obtained as a colorless oil. ¹H NMR (400 MHz, CDCl₃) δ 11.32 (br s, 1H), 5.68-5.44 (m, 2H), 3.12 (br d, J=6.8 Hz, 2H), 2.04-1.99 (m, 2H), 1.37-1.26 (m, 8H), 0.86 (t, J=6.7 Hz, 3H).

To a solution of Compound 11C (500 mg, 2.94 mmol) and N-Hydroxysuccinimide (700 mg, 6.08 mmol) in DMF (5 mL) and DCM (15 mL) was added EDCI (845 mg, 4.41 mmol) and HOAt (600 mg, 4.41 mmol). The mixture was stirred at 10° C. for 16 h. Saturated NH₄Cl (30 mL) was added and the aqueous layer was extracted with DCM (20 mL×2). The organic phase was washed with brine (50 mL). The organic phase was dried over Na₂SO₄, filtered and concentrated. The residue was purified by flash silica gel chromatography (ISCO®; 12 g SepaFlash® Silica Flash Column, Eluent of 20% Ethyl acetate/Petroleum ether gradient @ 30 mL/min). Compound 11D (450 mg, yield: 57.3% yield) was obtained as colorless oil. ¹H NMR (400 MHz, CDCl₃) δ 5.77-5.66 (m, 1H), 5.60-5.50 (m, 1H), 3.50-3.35 (m, 2H), 2.85 (br s, 4H), 2.11-2.05 (m, 2H), 1.43-1.26 (m, 8H), 0.89 (t, J=6.8 Hz, 3H).

To a solution of compound 3A (2.5 g, 5.95 mmol) in DCM (30 mL) was added TFA (135 mmol, 10 mL), the mixture was stirred at 15° C. for 1 h. The reaction mixture was concentrated to give a residue. Compound 11E (2.5 g, yield: 96.8%, TFA) as yellow oil was obtained, which was used into the next step without further purification. ¹H NMR (400 MHz, CDCl₃) δ 7.05 (d, J=8.3 Hz, 2H), 6.88-6.74 (m, 2H), 6.01-5.76 (m, 1H), 5.45-5.24 (m, 2H), 4.64 (d, J=6.0 Hz, 2H), 4.52-4.36 (m, 2H), 3.24-2.96 (m, 1H), 2.17 (d, J=5.8 Hz, 1H), 1.31-1.26 (m, 1H), 0.87 (dd, J=6.8, 12.3 Hz, 6H).

To a solution of compound 11C (500 mg, 2.94 mmol) and compound 11E (1.5 g, 3.45 mmol, TFA) in DMF (10 mL) was added HATU (1.23 g, 3.23 mmol) and DIEA (11.48 mmol, 2 mL), then the mixture was stirred at 12° C. for 1 h. The reaction mixture was diluted with water (60 mL), extracted with EA (30 mL×3), the organic layers were washed with water (50 mL×2), brine (50 mL×2), dried over Na₂SO₄, filtered and concentrated to give a residue. The residue was purified by flash silica gel chromatography (ISCO®; 4 g SepaFlash® Silica Flash Column, Eluent of 0-70% Ethyl acetate/Petroleum ether gradient @ 20 mL/min). Compound 3 (930 mg, yield: 60.6%) as light yellow oil was obtained. ¹H NMR (400 MHz, CDCl₃) δ 7.10-7.00 (m, 2H), 6.72 (d, J=8.3 Hz, 2H), 6.40-6.28 (m, 2H), 6.20-6.12 (m, 1H), 5.97-5.83 (m, 1H), 5.71-5.63 (m, 1H), 5.49-5.39 (m, 1H), 5.38-5.23 (m, 2H), 4.65-4.58 (m, 3H), 4.44 (dd, J=4.9, 8.4 Hz, 1H), 3.08-2.90 (m, 4H), 2.20-2.07 (m, 1H), 2.01-1.91 (m, 2H), 1.28-1.21 (m, 8H), 0.94-0.75 (m, 9H). MS (ESI) m/z (M+H)⁺ 473.2.

To a solution of compound 11F (930 mg, 1.97 mmol) in THF (15 mL) and H₂O (5 mL) was added LiOH.H₂O (330 mg, 7.86 mmol) at 0° C., the mixture was stirred at 0° C. for 2 h. The reaction mixture was concentrated to remove THF, diluted with water (30 mL), acidified with 1N HCl to pH˜3, extracted with EA (30 mL×3), the organic layers were dried over Na₂SO₄, filtered and concentrated to give a residue. Compound 11G (740 mg, yield: 80.9%) as light yellow solid was obtained, which was used into the next step without further purification. ¹H NMR (400 MHz, DMSO-d₆) δ 12.54 (s, 1H), 9.16-9.09 (m, 1H), 8.10-7.82 (m, 2H), 7.15-6.95 (m, 2H), 6.61 (d, J=8.3 Hz, 2H), 5.46-5.26 (m, 2H), 4.55-4.49 (m, 1H), 4.14 (dd, J=5.6, 8.6 Hz, 1H), 2.93-2.70 (m, 3H), 2.68-2.59 (m, 1H), 2.11-1.92 (m, 3H), 1.33-1.17 (m, 8H), 0.96-0.76 (m, 9H). MS (ESI) m/z (M+H)⁺ 433.3.

To a solution of compound 11G (740 mg, 1.71 mmol) and N-hydroxysuccinimide (395 mg, 3.43 mmol) in DCM (15 mL) and DMF (3 mL) was added EDCI (492 mg, 2.57 mmol) and HOAt (350 mg, 2.57 mmol) at 0° C., the mixture was stirred at 0° C. for 1 h, then stirred at 15° C. for 17 h. The reaction mixture was quenched with sat.NH₄Cl (50 mL), extracted with DCM (30 mL×2), the organic layers were washed with brine (50 mL×2), dried over Na₂SO₄, filtered and concentrated to give a residue. The residue was purified by flash silica gel chromatography (ISCO®; 12 g SepaFlash® Silica Flash Column, Eluent of 0-50% Ethyl acetate/Petroleum ether gradient @ 30 mL/min). Compound 11H (740 mg, yield: 77.9%) as light yellow oil was obtained. ¹H NMR (400 MHz, CDCl₃) δ 7.09-6.97 (m, 2H), 6.73 (d, J=8.6 Hz, 2H), 6.53-6.46 (m, 1H), 6.42 (d, J=7.8 Hz, 1H), 6.37-6.22 (m, 1H), 5.74-5.60 (m, 1H), 5.51-5.38 (m, 1H), 4.77 (dd, J=4.9, 8.6 Hz, 1H), 4.68-4.55 (m, 1H), 3.05-2.95 (m, 4H), 2.84 (s, 4H), 2.33-2.22 (m, 1H), 2.02-1.92 (m, 2H), 1.26 (q, J=7.3 Hz, 8H), 0.96 (t, J=6.4 Hz, 6H), 0.90-0.84 (m, 3H).

### (Z)—N—((S)-3-(4-hydroxyphenyl)-1-(((S)-1-(((3S,6S,11R,E)-6-isopropyl-3-(4-methoxybenzyl)-2,5,8-trioxo-1-oxa-4,7-diazacyclododec-9-en-11-yl)amino)-3-methyl-1-oxobutan-2-yl)amino)-1-oxopropan-2-yl)dec-3-enamide (11)

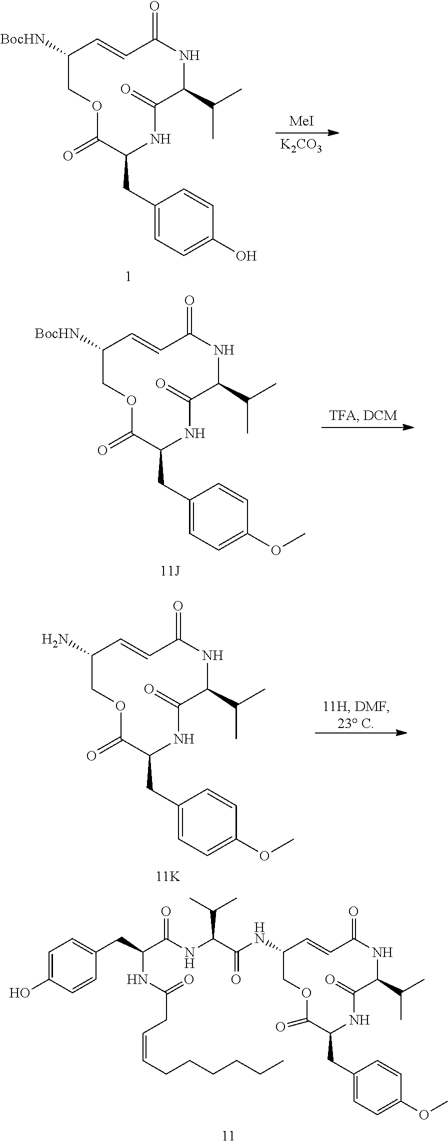

To a solution of compound 1 (50 mg, 105.15 umol) in DMF (2 mL) was added K₂CO₃ (29.06 mg, 210.29 umol) and Mel (298.48 mg, 2.10 mmol, 130.91 uL). The mixture was stirred at 20° C. for 48 h. Water (30 mL) was added and the aqueous layer was extracted with EA (20 mL×2). The organic phase was washed with brine (50 mL). The organic phase was dried over Na₂SO₄, filtered and concentrated. The residue was purified by preparatory-TLC (SiO₂, DCM:EA=1:1). Compound 11J (40 mg, yield: 69.9%) was obtained as a white solid. ¹H NMR (400 MHz, Methanol-d₄) δ 7.09 (br d, J=8.3 Hz, 2H), 6.79 (br d, J=8.3 Hz, 2H), 6.45 (dd, J=4.2, 15.7 Hz, 1H), 6.25 (br d, J=15.4 Hz, 1H), 4.67 (br d, J=10.3 Hz, 1H), 4.61-4.54 (m, 1H), 4.47 (br s, 1H), 3.81 (br d, J=6.1 Hz, 2H), 3.73 (s, 3H), 3.18-3.00 (m, 2H), 1.87-1.76 (m, 1H), 1.43 (s, 9H), 0.87 (d, J=6.8 Hz, 3H), 0.75 (br d, J=6.8 Hz, 3H). MS (ESI) m/z (M+Na)⁺ 512.2.

To a solution of compound 11J (40 mg, 81.71 umol) in DCM (5 mL) was added TFA (1.54 g, 13.51 mmol, 1 mL). The mixture was stirred at 20° C. for 3 h. Saturated aqueous NaHCO₃ (20 mL) was added and the aqueous layer was extracted with CH₂Cl₂ (20 mL×3). The combined organic phase was dried over Na₂SO₄, filtered and concentrated. The residue was used to the next step without purification. Compound 11K (25 mg, yield: 78.6%) was obtained as a white solid. MS (ESI) m/z (M+H)⁺ 390.2.

To a solution of compound 11K (25 mg, 64.19 umol) in DMF (2 mL) was added Compound 11H (71.43 mg, 134.87 umol). The mixture was stirred at 10° C. for 16 h, and then heated to 25° C. for 24 h. The reaction was quenched with saturated NH₄Cl (10 mL), and then H₂O (20 mL) was added and the aqueous layer was extracted with EA (15 mL×3). The organic phase was washed with brine (30 mL). The organic phase was dried over Na₂SO₄, filtered and concentrated. The residue was purified by preparatory-TLC (SiO₂, EA). Compound 11 (25 mg, yield: 46.9%) was obtained as a white solid. ¹H NMR (400 MHz, DMSO-d₆) δ 9.12 (s, 1H), 8.84 (br s, 1H), 8.45-8.24 (m, 2H), 7.98-7.83 (m, 1H), 7.10-7.00 (m, 5H), 6.77 (d, J=8.6 Hz, 2H), 6.67-6.57 (m, 1H), 6.54 (d, J=8.6 Hz, 2H), 6.23 (dd, J=4.8, 15.8 Hz, 1H), 5.42-5.21 (m, 2H), 4.86 (br s, 1H), 4.44-4.32 (m, 2H), 4.17 (t, J=8.4 Hz, 1H), 3.99 (br s, 2H), 3.67 (s, 3H), 3.58 (br d, J=10.5 Hz, 1H), 3.22-3.15 (m, 1H), 3.10-3.02 (m, 1H), 2.94-2.85 (m, 1H), 2.79 (br d, J=6.1 Hz, 2H), 2.55 (br d, J=12.7 Hz, 1H), 2.12-2.02 (m, 1H), 1.94-1.84 (m, 2H), 1.65 (br dd, J=6.6, 13.0 Hz, 1H), 1.20 (br s, 8H), 0.81 (br d, J=6.6 Hz, 9H), 0.73 (d, J=6.6 Hz, 3H), 0.63 (d, J=6.6 Hz, 3H). MS (ESI) m/z (M+H)⁺ 804.5.

### Example 9

### Compound 10

### (Z)—N-(2-(((S)-1-(((3S,6S,11R,E)-3-(4-hydroxybenzyl)-6-isopropyl-2,5,8-trioxo-1-oxa-4,7-diazacyclododec-9-en-11-yl)amino)-3-methyl-1-oxobutan-2-yl)amino)-2-oxoethyl)dec-3-enamide (10)

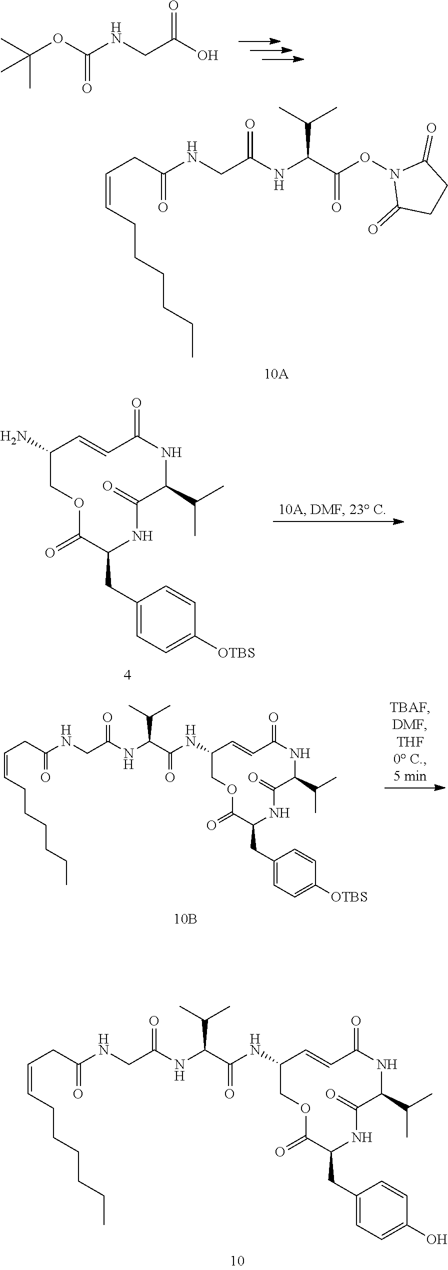 [SMILES: CC(C)(C)OC(=O)NCC(=O)O ; CCCCCC/C=C\CC(=O)NCC(=O)N[C@H](C(=O)ON1C(=O)CCC1=O)C(C)C ; CC(C)[C@@H]1NC(=O)/C=C/[C@@H](N)COC(=O)[C@H](Cc2ccc(O[Si](C)(C)C(C)(C)C)cc2)NC1=O ; CCCCCC/C=C\CC(=O)NCC(=O)N[C@H](C(=O)N[C@@H]1/C=C/C(=O)N[C@@H](C(C)C)C(=O)N[C@@H](Cc2ccc(O[Si](C)(C)C(C)(C)C)cc2)C(=O)OC1)C(C)C ; CCCCCC/C=C\CC(=O)NCC(=O)N[C@H](C(=O)N[C@@H]1/C=C/C(=O)N[C@@H](C(C)C)C(=O)N[C@@H](Cc2ccc(O)cc2)C(=O)OC1)C(C)C]

(Tert-butoxycarbonyl)glycine was converted using same procedures as for intermediates 3A and 11H to obtain intermediates 10A. Compound 10A (700 mg, yield: 76.3%) was obtained as colorless oil. ¹H NMR (400 MHz, DMSO-d₆) δ 8.48-8.37 (m, 1H), 8.12-7.92 (m, 1H), 5.53-5.36 (m, 2H), 4.57 (dd, J=6.0, 8.2 Hz, 1H), 3.88-3.77 (m, 1H), 3.76-3.64 (m, 1H), 2.97-2.83 (m, 2H), 2.78 (br s, 4H), 2.21-2.08 (m, 1H), 2.02-1.94 (m, 2H), 1.33-1.17 (m, 8H), 0.96 (dd, J=3.3, 6.7 Hz, 6H), 0.86-0.78 (m, 3H). MS (ESI) m/z (M+H)⁺ 424.3.

Intermediate 10A and compound 4 were subjected to synthetic procedures as for compound 3 to yield the compound 10. Compound 10 (35 mg, yield: 49.0%) as a white solid was obtained. ¹H NMR (400 MHz, Methanol-d₄) δ 7.02 (d, J=8.3 Hz, 2H), 6.68 (d, J=8.6 Hz, 2H), 6.53-6.39 (m, 1H), 6.21 (d, J=15.2 Hz, 1H), 5.64-5.42 (m, 2H), 4.86-4.46 (m, 3H), 4.33-4.21 (m, 1H), 4.02-3.92 (m, 2H), 3.90-3.78 (m, 2H), 3.19-2.96 (m, 4H), 2.25-2.16 (m, 1H), 2.13-2.00 (m, 2H), 1.94-1.79 (m, 1H), 1.41-1.24 (m, 9H), 1.00-0.88 (m, 12H), 0.81 (d, J=6.8 Hz, 3H). MS (ESI) m/z (M+H)⁺ 684.1.

### Example 10

### Compound 12-14

### tert-butyl ((S)-1-(((3S,6S,11R,E)-3-(4-hydroxybenzyl)-6-isopropyl-2,5,8-trioxo-1-oxa-4,7-diazacyclododec-9-en-11-yl)amino)-4-methyl-1-oxopentan-2-yl)carbamate (12)

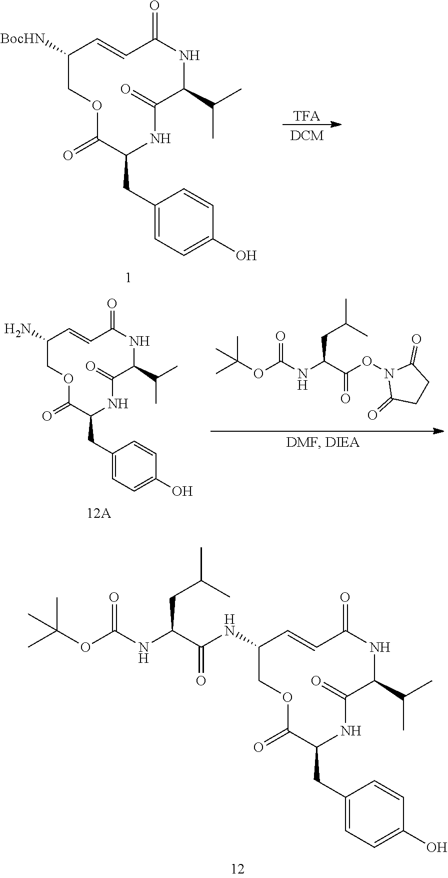

To a solution of compound 1 (250 mg, 525.73 umol) in DCM (8 mL) was added TFA (3.08 g, 27.01 mmol, 2 mL). The mixture was stirred at 15° C. for 3 h. The reaction was concentrated. The residue was used to the next step without purification. Compound 12A (257 mg, crude, TFA) was obtained as a yellow solid. ¹H NMR (400 MHz, Methanol-d₄) δ 6.99 (br d, J=8.3 Hz, 1H), 6.72-6.63 (m, 1H), 6.45 (br s, 2H), 4.71 (br d, J=11.0 Hz, 1H), 4.36 (br s, 2f), 3.98 (br d, J=12.5 Hz, 1H), 3.88 (d, J=5.4 Hz, 1H), 3.22-3.00 (m, 2H), 1.96-1.80 (m, 1H), 0.89 (br d, J=6.8 Hz, 3H), 0.81 (br d, J=6.6 Hz, 3H). MS (ESI) m/z (M+H)⁺ 376.2.

To a solution of compound 12A (50 mg, 102.16 umol, TFA) and (2,5-dioxopyrrolidin-1-yl) (2S)-2-(tert-butoxycarbonylamino)-4-methyl-pentanoate (67 mg, 204.31 umol) in DMF (3 mL) was added DIEA (510.79 umol, 0.09 mL), the mixture was stirred at 15° C. for 1 h. The reaction mixture was diluted with water (20 mL), extracted with EA (10 mL×2), the organic layers were washed with brine (20 mL), dried over Na₂SO₄, filtered and concentrated to give a residue. The residue was purified by preparatory-TLC (EA:MeOH=30:1). Compound 12 (40 mg, yield: 65.2%) as white solid was obtained. ¹H NMR (400 MHz, Methanol-d₄) δ 7.08-6.98 (m, 2H), 6.75-6.63 (m, 2H), 6.57-6.39 (m, 1H), 6.16 (d, J=14.8 Hz, 1H), 4.85-4.49 (m, 3H), 4.19 (d, J=7.0 Hz, 1H), 3.90-3.70 (m, 2H), 3.18-2.87 (m, 2H), 1.93-1.79 (m, 11H), 1.76-1.63 (m, 1H), 1.60-1.50 (m, 2H), 1.47-1.40 (m, 9H), 1.00-0.95 (m, 6H), 0.91 (d, J=6.8 Hz, 3H), 0.79 (d, J=6.8 Hz, 3H). MS (ESI) m/z (M+Na)⁺ 611.0.

### tert-butyl ((s)-1-(((3S,6S,11R,E)-3-(4-hydroxybenzyl)-6-isopropyl-2,5,8-trioxo-1-oxa-4,7-diazacyclododec-9-en-11-yl)amino)-1-oxopropan-2-yl)carbamate (13)

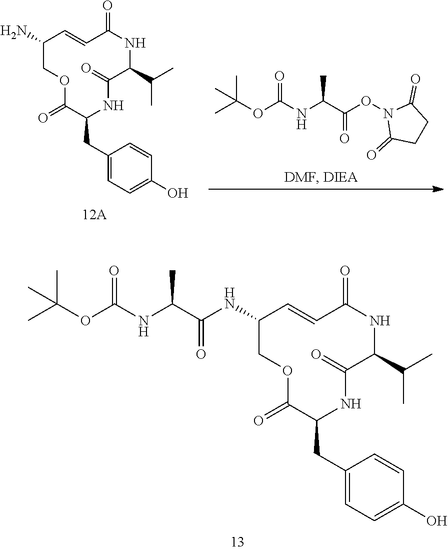 [SMILES: CC(C)[C@@H]1NC(=O)/C=C/[C@@H](N)COC(=O)[C@H](Cc2ccc(O)cc2)NC1=O ; C[C@H](NC(=O)OC(C)(C)C)C(=O)ON1C(=O)CCC1=O ; CC(C)[C@@H]1NC(=O)/C=C/[C@@H](NC(=O)[C@H](C)NC(=O)OC(C)(C)C)COC(=O)[C@H](Cc2ccc(O)cc2)NC1=O]

To a solution of compound 12A (80 mg, 163.45 umol, TFA) and (2,5-dioxopyrrolidin-1-yl) (2S)-2-(tert-butoxycarbonylamino)propanoate (93.59 mg, 326.90 umol) in DMF (3 mL) was added DIEA (74.20 mg, 574.11 umol, 0.1 mL). The mixture was stirred at 15° C. for 1 h. The reaction was added H₂O (20 mL), and extracted with EA (10 mL×3), the phase was washed with brine (30 mL), dried over Na₂SO₄, filtered and concentrated. The residue was purified by preparatory-TLC (SiO₂, EA:MeOH=15:1). Compound 13 (25 mg, yield: 27.7%) was obtained as a white solid. ¹H NMR (400 MHz, DMSO-d₆) δ 9.10 (s, 1H), 8.43 (br d, J=7.3 Hz, 1H), 7.84 (br d, J=8.1 Hz, 1H), 7.03 (br d, J=5.4 Hz, 1H), 6.96-6.85 (m, 3H), 6.59 (br d, J=8.3 Hz, 2H), 6.21 (s, 2H), 4.69 (br d, J=6.4 Hz, 1H), 4.43 (br d, J=11.0 Hz, 1H), 4.15 (br s, 1H), 4.04 (br s, 1H), 3.74-3.59 (m, 2H), 2.99 (br s, 2H), 1.83-1.66 (m, 1H), 1.33 (s, 9H), 1.16 (br d, J=6.8 Hz, 3H), 0.79 (br d, J=6.6 Hz, 3H), 0.68 (br d, J=6.6 Hz, 3H). MS (ESI) m/z (M+Na)⁺ 569.0.

### tert-butyl ((s)-1-(((3S,6S,11R,E)-3-(4-hydroxybenzyl)-6-isopropyl-2,5,8-trioxo-1-oxa-4,7-diazacyclododec-9-en-11-yl)amino)-1-oxo-3-phenylpropan-2-yl)carbamate (14)

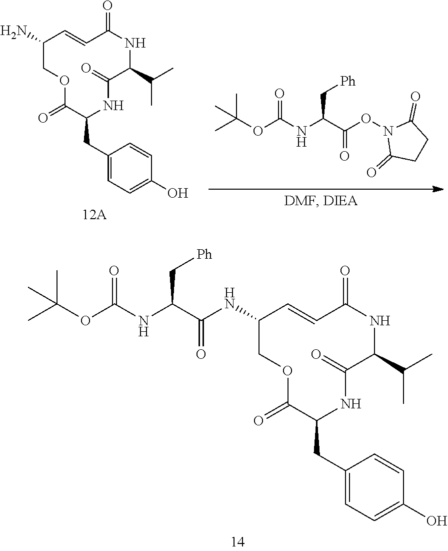 [SMILES: CC(C)[C@@H]1NC(=O)/C=C/[C@@H](N)COC(=O)[C@H](Cc2ccc(O)cc2)NC1=O ; CC(C)(C)OC(=O)N[C@@H](Cc1ccccc1)C(=O)ON1C(=O)CCC1=O ; CC(C)[C@@H]1NC(=O)/C=C/[C@@H](NC(=O)[C@H](Cc2ccccc2)NC(=O)OC(C)(C)C)COC(=O)[C@H](Cc2ccc(O)cc2)NC1=O]

To a solution of compound 12A (70 mg, 143.02 umol, TFA) and (2,5-dioxopyrrolidin-1-yl) (2S)-2-(tert-butoxycarbonylamino)-3-phenyl-propanoate (104 mg, 286.04 umol) in DMF (3 mL) was added DEA (715.10 umol, 0.12 mL), the mixture was stirred at 15° C. for 1 h. The reaction mixture was diluted with water (30 mL), extracted with EA (10 mL×3), the organic layers were washed with brine (20 mL), dried over Na₂SO₄, filtered and concentrated to give a residue. The residue was purified by preparatory-TLC (DCM:MeOH=10:1). Compound 14 (15 mg, yield: 15.5%) as white solid was obtained. ¹H NMR (400 MHz, DMSO-d₆) δ 9.17 (s, 1H), 8.73 (s, 1H), 8.26 (d, J=8.6 Hz, 1H), 7.31 (d, J=7.8 Hz, 2H), 7.21 (t, J=7.6 Hz, 2H), 7.16-7.01 (m, 3H), 6.96 (d, J=8.3 Hz, 2H), 6.62 (d, J=8.6 Hz, 2H), 6.18 (s, 2H), 4.78 (d, J=8.1 Hz, 1H), 4.47-4.41 (m, 1H), 4.36-4.27 (m, 1H), 4.05 (s, 1H), 3.83-3.76 (m, 1H), 3.63 (d, J=9.0 Hz, 1H), 3.17-3.08 (m, 1H), 3.05-2.97 (m, 2H), 2.85-2.74 (m, 1H), 1.79-1.68 (m, 1H), 1.29 (s, 9H), 0.87-0.81 (m, 3H), 0.71 (d, J=6.6 Hz, 3H). MS (ESI) m/z (M+Na)⁺ 645.2.

### Example 11

### Compound 15-17

### tert-butyl ((3S,6S,11R,E)-6-isopropyl-3-methyl-2,5,8-trioxo-1-oxa-4,7-diazacyclododec-9-en-11-yl)carbamate (15

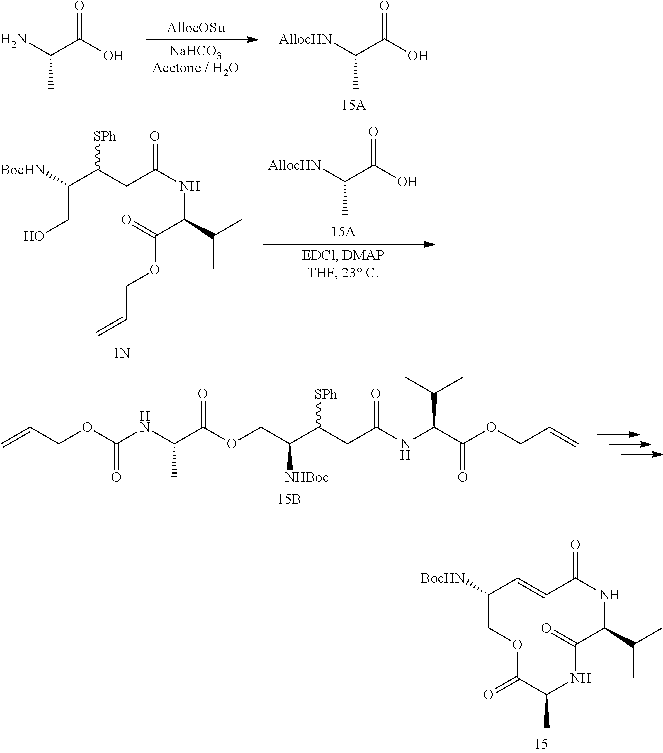

To a solution of L-alanine (5 g, 56.12 mmol) in acetone (80 mL) and H₂O (80 mL) was added NaHCO₃ (14.14 g, 168.36 mmol), then allyl (2,5-dioxopyrrolidin-1-yl) carbonate (12.29 g, 61.73 mmol) was added, the mixture was stirred at 10° C. for 18 h. The reaction was filtered, and the filtrate was extracted with EA (50 mL×2), the water phase was adjusted with HCl (1N) to pH˜3, and then extracted with EA (50 mL×3), the organic phase was dried over Na₂SO₄, filtered, and concentrated to give a residue. Compound 15A (7.9 g, yield: 81.3%) as colorless oil was obtained, which was used into the next step without further purification. ¹H NMR (400 MHz, DMSO-d₆) δ 12.53 (s, 1H), 7.56 (d, J=7.5 Hz, 1H), 6.02-5.77 (m, 1H), 5.39-5.11 (m, 2H), 4.47 (d, J=5.3 Hz, 2H), 4.08-3.89 (m, 1H), 1.25 (d, J=7.3 Hz, 3H).

Intermediates 1N and 15A were subjected to same conditions as used in synthesis of compound 5 to yield the compound 15. Compound 15 (90 mg, yield: 45.4%) as a light yellow solid was obtained. ¹H NMR (400 MHz, DMSO-d₆) δ 8.41 (d, J=7.1 Hz, 1H), 7.20 (d, J=6.4 Hz, 1H), 6.82 (d, J=8.6 Hz, 1H), 6.42-6.31 (m, 1H), 6.42-6.31 (m, 1H), 6.28-6.16 (m, 1H), 4.50 (d, J=5.4 Hz, 1H), 4.23 (d, J=10.3 Hz, 1H), 3.94-3.87 (m, 1H), 3.76-3.64 (m, 2H), 1.90-1.87 (m, 1H), 1.41 (s, 9H), 1.34 (d, J=7.1 Hz, 3H), 0.96 (d, J=6.6 Hz, 3H), 0.90 (d, J=6.8 Hz, 3H) MS (ESI) m/z (M+Na)+406.1.

### (Z)—N—((S)-3-(4-hydroxyphenyl)-1-(((S)-1-(((3S,6S,11R,E)-6-isopropyl-3-methyl-2,5,8-trioxo-1-oxa-4,7-diazacyclododec-9-en-11-yl)amino)-3-methyl-1-oxobutan-2-yl)amino)-1-oxopropan-2-yl)dec-3-enamide (16)

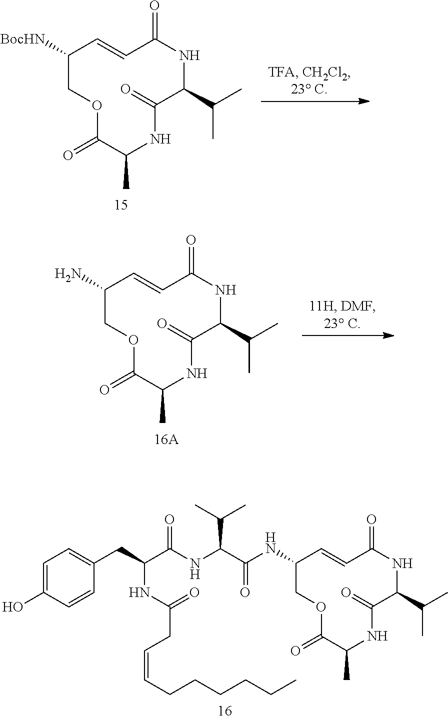 [SMILES: CC(C)[C@@H]1NC(=O)/C=C/[C@@H](NC(=O)OC(C)(C)C)COC(=O)[C@H](C)NC1=O ; CC(C)[C@@H]1NC(=O)/C=C/[C@@H](N)COC(=O)[C@H](C)NC1=O ; CCCCCC/C=C\CC(=O)N[C@@H](Cc1ccc(O)cc1)C(=O)N[C@H](C(=O)N[C@@H]1/C=C/C(=O)N[C@@H](C(C)C)C(=O)N[C@@H](C)C(=O)OC1)C(C)C]

Intermediate 15 was subjected to same conditions as used in synthesis of compound 11 to yield the compound 16. Compound 16 (95 mg, yield: 53.1%) as a white solid was obtained. ¹H NMR (400 MHz, Methanol-d₄) δ 7.13-6.99 (m, 2H), 6.68 (dd, J=5.3, 8.5 Hz, 2H), 6.54-6.12 (m, 2H), 5.63-5.32 (m, 2H), 4.85-4.30 (m, 4H), 4.30-4.15 (m, 1H), 3.87-3.70 (m, 2H), 3.18-2.59 (m, 4H), 2.30-1.85 (m, 4H), 1.43-1.24 (m, 11H), 1.14-0.99 (m, 6H), 0.98-0.87 (m, 9H). MS (ESI) m/z (M+H)⁺ 698.2.

### Tert-butyl ((S)-3-(4-hydroxyphenyl)-1-(((S)-1-(((3S,6S,11R,E)-6-isopropyl-3-methyl-2,5,8-trioxo-1-oxa-4,7-diazacyclododec-9-en-11-yl)amino)-3-methyl-1-oxobutan-2-yl)amino)-1-oxopropan-2-yl)carbamate (17)

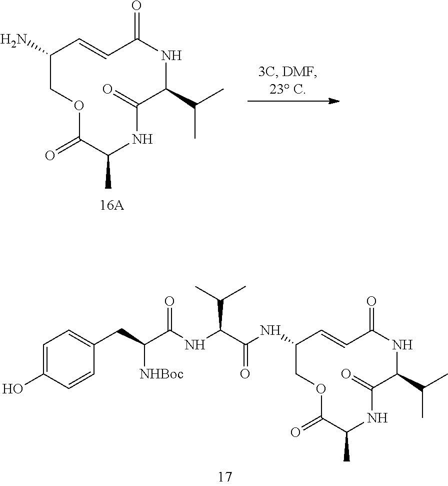 [SMILES: CC(C)[C@@H]1NC(=O)/C=C/[C@@H](N)COC(=O)[C@H](C)NC1=O ; CC(C)[C@@H]1NC(=O)/C=C/[C@@H](NC(=O)[C@@H](NC(=O)[C@H](Cc2ccc(O)cc2)NC(=O)OC(C)(C)C)C(C)C)COC(=O)[C@H](C)NC1=O]

Intermediate 16A and 3C were subjected to same conditions as used in synthesis of compound 11 to yield the compound 17. Compound 17 (15 mg, yield: 8.7%) as a white solid was obtained. ¹H NMR (400 MHz, DMSO-d₆) δ 9.18 (d, J=3.8 Hz, 1H), 8.80 (s, 1H), 8.39-7.91 (m, 2H), 7.81-6.91 (m, 4H), 6.71-6.45 (m, 3H), 6.34-6.06 (m, 1H), 4.99-4.73 (m, 1H), 4.48-4.20 (m, 3H), 4.10 (s, 1H), 4.01-3.80 (m, 1H), 3.70-3.55 (m, 2H), 2.83 (s, 1H), 2.11-1.73 (m, 2H), 1.37-1.21 (m, 12H), 0.94-0.79 (m, 12H). MS (ESI) m/z (M−Boc+H)⁺ 546.0.

### Example 12

### Compound 18

### (Z)—N—((S)-1-(((S)-1-(((3S,6S,11R,E)-3-(4-hydroxybenzyl)-6-isopropyl-2,5,8-trioxo-1-oxa-4,7-diazacyclododec-9-en-11-yl)amino)-3-methyl-1-oxobutan-2-yl)amino)-3-(4-hydroxyphenyl)-1-oxopropan-2-yl)dec-3-enamide (13)

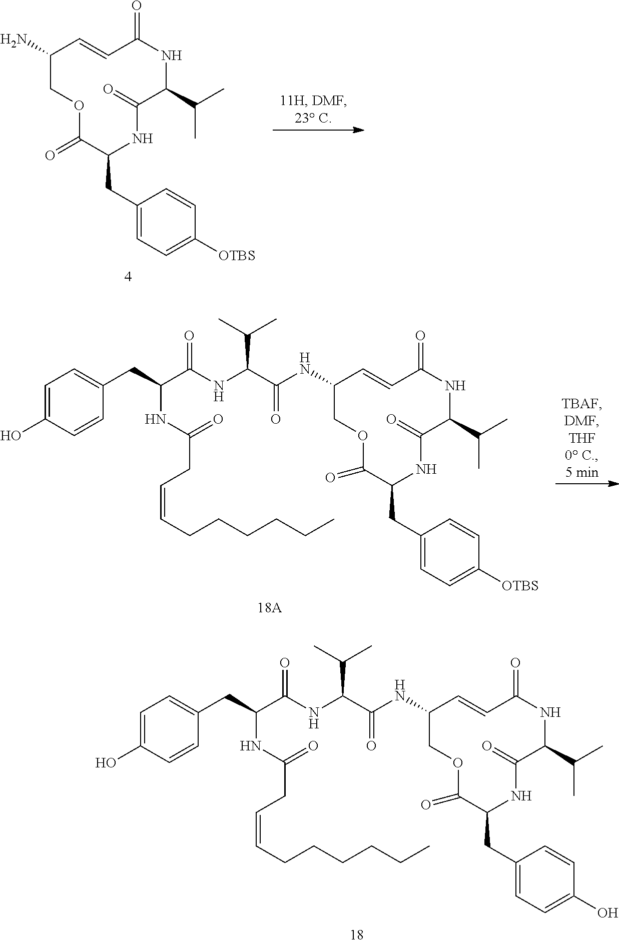 [SMILES: CC(C)[C@@H]1NC(=O)/C=C/[C@@H](N)COC(=O)[C@H](Cc2ccc(O[Si](C)(C)C(C)(C)C)cc2)NC1=O ; CCCCCC/C=C\CC(=O)N[C@@H](Cc1ccc(O)cc1)C(=O)N[C@H](C(=O)N[C@@H]1/C=C/C(=O)N[C@@H](C(C)C)C(=O)N[C@@H](Cc2ccc(O[Si](C)(C)C(C)(C)C)cc2)C(=O)OC1)C(C)C ; CCCCCC/C=C\CC(=O)N[C@@H](Cc1ccc(O)cc1)C(=O)N[C@H](C(=O)N[C@@H]1/C=C/C(=O)N[C@@H](C(C)C)C(=O)N[C@@H](Cc2ccc(O)cc2)C(=O)OC1)C(C)C]

Intermediate 11H and compound 4 were subjected to same conditions as used in synthesis of compound 10 to yield the compound 18. Compound 18 (20 mg, yield: 94%) as a white solid was obtained. [α]_{D}²³ −25.603 (c 1.0, CHCl₃). ¹H NMR (600 MHz, dmso-d₅) δ (ppm): 8.42 (s, 1H), 7.98-7.92 (m, 3H) 7.07 (s, 1H), 6.97 (s, 1H), 6.99 (d, J=12 Hz, 2H), 6.96 (d, J=12 Hz, 2H), 6.71 (s, 1H), 6.62 (d, J=12 Hz, 2H), 6.59 (d, J=12 Hz, 2H), 6.32 (dd, 2H), 6.27 (d, 2H), 5.43-5.32 (m, 2H), 4.78 (s, 1H), 4.45 (m, 2H), 4.22 (m, 1H), 4.16 (d, 1H), 3.69-3.68 (m, 2H), 3.05-3.02 (m, 2H), 2.91 (d, 1H), 2.85 (d, 2H), 2.65 (m, 1H), 2.00 (m, 1H), 1.94 (m, 2H), 1.77 (m, 1H), 1.30-1.20 (m, 8H), 0.88-0.78 (m, 12H), 0.7 (d, 3H).

### Biological Data

### Example 13

Calpain 1, 2, and 9 activity and inhibition thereof was assessed by means of a continuous fluorescence assay. The SensoLyte 520 Calpain substrate (Anaspec Inc.) was optimized for detecting calpain activity. This substrate contains an internally quenched 5-FAM/QXLTM 520 FRET pair. Calpains 1, 2, and 9 cleave the FRET substrate into two separate fragments resulting in an increase of 5-FAM fluorescence that is proportional to calpain activity

Assays were typically setup in black 384-well plates using automated liquid handling as follows. Calpain assay base buffer typically contains 50 mM Tris, pH 7.5, 100 mM NaCl and 1 mM DTT. Inhibitors were serially diluted in DMSO and used to setup 2× mixtures with calpains in the aforementioned buffer. After incubation at ambient temperature (25C), the reaction was initiated by adding a 2× mix of the fluorescent peptide substrate and CaCl₂) (required for in-situ calpain activation) in the same buffer. Reaction progress curve data were typically collected for 10 min using excitation/emission wavelengths of 490 nm/520 nm on SpectraMax i3x or the FLIPR-Tetra plate readers (Molecular Devices Inc.). Reaction rates were calculated from progress curve slopes typically over 1-5 min. Dose response curves (rate vs. log inhibitor concentration) were typically fit to a 4-parameter logistic function to extract IC50 values.

### Example 14

Cathepsin activity and inhibition thereof was assessed by means of a continuous fluorescence assay. All assays were run in 384 well format and utilized peptide substrates that liberate fluorescence upon protease catalyzed cleavage. FLIPR plate readers (Molecular Devices Inc.) were used to monitor reactions and extract initial rates which were then fit (four parameter logistic function) to obtain IC50 values. All enzymes are human. All enzymes and reagents were obtained commercially and are summarized in Tables 1 and 2 below along with assay conditions.

TABLE 1

| | Enzyme | | Substrate |
|---|---|---|---|
| | Conc | | Conc |
| Enzyme | (nM) | Substrate | (uM) |
| Cathepsin B | 25 | QXL520/HiLyte Fluor488 CatB | 1 |
| Cathepsin K | 10 | QXL520/HiLyte Fluor488 CatK | 1 |
| Cathepsin L | 0.025 | QXL ™ 520/HiLyte | 1 |
| | | Fluor ™ 488 CatL | |
| Cathepsin S | 6 | 5-FAM/QXL520 Cats | 1 |

All enzymes were pre-incubated with inhibitor for 30 minutes prior to substrate addition

TABLE 2

| Enzyme | Assay Buffer |
|---|---|
| Cathepsin B | 25 mM MES, 5 mM DTT, pH 5.0 |
| Cathepsin K | 50 mM NaOAc, pH 5.5, 2.5 mM EDTA, |
| | 1 mM DTT, 0.01% Triton X-100 |
| Cathepsin L | 50 mM MES, 5 mM DTT, 1 mM EDTA, |
| | 0.005% (w/v) Brij-35, pH 6.0 |
| Cathepsin S | 50 mM NaOAc, 5 mM DTT, 250 mM NaCl, |
| | pH 4.5 |

All enzymes were pre-incubated with inhibitor for 30 minutes prior to substrate addition

### Calpain Inhibition

TABLE 3
Calpain inhibition assay

Column A: Human Calpain 1/NS1 IC50
Column B: Human Calpain 2/NS1 IC50
Column C: Human Calpain 9/NS1 IC50
Column D: Human Cathepsin B mean IC50
Column E: Human Cathepsin K mean IC50
Column F: Human Cathepsin S mean IC50
Column G: Human Cathepsin L mean IC50

TABLE 3

| Compound | Column | Column | Column | Column | Column | Column | Column |
|---|---|---|---|---|---|---|---|
| No. | A | B | C | D | E | F | G |
| 1 | C | C | C | A | A | A | A |
| 2 | B | A | C | A | A | A | A |
| 3 | A | A | C | A | A | A | A |
| 4 | C | C | C | C | A | B | B |
| 5 | C | C | C | B | A | A | A |
| 6 | A | A | C | A | A | A | A |
| 7 | B | B | C | A | A | A | A |
| 8 | C | C | C | A | A | A | A |
| 9 | B | A | C | A | A | A | A |
| 10 | A | A | C | A | A | A | A |
| 11 | A | A | C | A | A | A | A |
| 12 | A | A | C | A | A | A | A |
| 13 | C | C | C | A | A | A | A |
| 14 | C | C | C | B | A | A | A |
| 15 | C | C | C | B | A | A | A |
| 16 | A | A | C | A | A | A | A |
| 17 | A | A | C | A | A | A | A |
| 18 | A | A | C | A | A | A | A |

A: <3 uM;
B: 3-10 uM;
C: >10 uM;
ND: Not Determined

### Carbon Tetrachloride-Induced Liver Fibrosis in Mice or Rats

Carbon tetrachloride-induced liver fibrosis is a widely used and accepted model for evaluating novel antifibrotic therapies. The methods for inducing liver fibrosis by carbon tetrachloride administration is described in Lee, J Clin Invest, 1995 and Tsukamoto, Semin Liver Dis, 1990. Briefly, male C57BL/6 mice are challenged with 1 mg/kg carbon tetrachloride (Sigma Aldrich, diluted 1:7 in corn or olive oil) administered by intraperitoneal injection twice weekly for a period of 4 weeks. Mice are euthanized on day 28. In an alternative implementation, Wistar rats are administered carbon tetrachloride by intraperitoneal injection three times per week for 8-12 weeks. Rats are euthanized at the termination of the experiment, 8-12 after study initiation.

Blood is collected by cardiac puncture and processed into serum for evaluation of liver enzymes (including ALT, AST, ALP, etc.) at several timepoints throughout the study and at termination of the study. The liver tissues from all animals are collected and fixed by immersion in 10% neutral buffered formalin, processed, paraffin embedded, sectioned, mounted, and stained with Masson's Trichrome (Tri) or Picrosirius Red (PSR) using standard histological methods for evaluation of fibrosis severity.

### Mouse Unilateral Ureteral Obstruction Kidney Fibrosis Model

Female C57BL/6 mice (Harlan, 4-6 weeks of age) will be given free access to food and water and allowed to acclimate for at least 7 days prior to test initiation. After acclimation, mice are anesthetized and undergo unilateral ureteral obstruction (UUO) surgery or sham to left kidney. Briefly, a longitudinal, upper left incision is performed to expose the left kidney. The renal artery is located and 6/0 silk thread is passed between the artery and the ureter. The thread is looped around the ureter and knotted 3 times insuring full ligation of ureter. The kidney is returned to abdomen, the abdominal muscle is sutured and the skin is stapled closed. All animals are euthanized 4, 8, 14, 21, or 28 days after UUO surgery. Following sacrifice blood is collected via cardiac puncture, the kidneys are harvested and one half of the kidney is frozen at −80° C. and the other half is fixed in 10% neutral buffered formalin for histopathological assessment of kidney fibrosis.

### Bleomycin Dermal Fibrosis Model

Bleomycin (Calbiochem, Billerica Mass.) is dissolved in phosphate buffered saline (PBS) at 10 ug/ml, and sterilized by filtration. Bleomycin or PBS control is injected subcutaneously into two locations on the shaved back of C57/BL6 or S129 mice (Charles River/Harlan Labs, 20-25 g) once daily for 28 days while under isoflourane anesthesia (5% in 100% 02). After 28 days, mice are euthanized and 6 mm-full thickness punch biopsies are obtained from each injection site. Dermal fibrosis is assessed by standard histopathology and hydroxyproline biochemical assays.

### Example 15: Targeting Calpain

### Inhibition of EpMT

For assessment of in vitro EMT, NMuMG cells (ATCC) are grown to confluence in 10% serum (Fetal Bovine Serum) growth media (Dubecco's Modified Eagles Medium supplemented with 10 ug/mL insulin) and then are followed by 24 h starvation in 0.5% serum media+/−drug inhibitors. Cells are then treated with recombinant human TGFb1 (R&D Systems 5 ng/mL)+/−drug inhibitors in 0.5% serum media. For time points greater than 24 h, the aforementioned media is refreshed every 24 hours. Cell lysates were analyzed for aSMA protein expression by western blot.

Miettinen et al. (1994). “TGF-beta induced transdifferentiation of mammary epithelial cells to mesenchymal cells: involvement of type I receptors.” J Cell Biol 127(6 Pt 2):2021-36.

Lamouille et al. (2014). “Molecular mechanisms of epithelial-mesenchymal transition.” Nat Rev Mol Cell Biol 15(3):178-96.

For assessment of in vitro FMT, Normal Human Lung Fibroblasts (NHLF) cells (Lonza) were grown in Fibroblast Growth Media-2 (Lonza CC-3131/with CC-4126 bullet kit) and then were followed by 24 h starvation in serum/growth factor free Fibroblast Basal Media-2 (Lonza CC-3131)+/−drug inhibitors. Cells were then treated with TGFb1 (5 ng/mL) Fibroblast Basal Media+/−drug inhibitors. Cell lysates are analyzed for aSMA protein expression by western blot.

Further details may be found in Pegorier et al. (2010). “Bone Morphogenetic Protein (BMP)-4 and BMP-7 regulate differentially Transforming Growth Factor (TGF)—B1 in normal human lung fibroblasts (NHLF)” Respir Res 11:85, which is incorporated herein by reference in its entirety.

### Example 16: Human Treatment

The efficacy of treatment with a compound of a preferred embodiment compared with placebo in patients with idiopathic pulmonary fibrosis (IPF) and the safety of treatment with a compound of a preferred embodiment compared with placebo in patients with IPF is assessed. The primary outcome variable is the absolute change in percent predicted forced vital capacity (FVC) from baseline to Week 52. Other possible end-points would include, but are not limited to: mortality, progression free survival, change in rate of FVC decline, change in Sp02, and change in biomarkers (HRCT image analysis; molecular and cellular markers of disease activity). Secondary outcome measures include: composite outcomes of important IPF-related events; progression-free survival; the rate of death from any cause; the rate of death from IPF; categorical assessment of absolute change in percent predicted FVC from baseline to Week 52; change in Shortness-of-Breath from baseline to Week 52; change in percent predicted hemoglobin (Hb)-corrected carbon monoxide diffusing capacity (DLco) of the lungs from baseline to Week 52; change in oxygen saturation during the 6 minute walk test (6MWT) from baseline to Week 52; change in high-resolution computed tomography (HRCT) assessment from baseline to Week 52; change in distance walked in the 6MWT from baseline to Week 52. Patients eligible for this study include, but are not limited to: those patients that satisfy the following inclusion criteria: diagnosis of IPF; 40 to 80 years of age; FVC≥50% predicted value; DLco≥35% predicted value; either FVC or DLco≤90% predicted value; no improvement in past year; a ratio of the forced expiratory volume in 1 second (FEV1) to the FVC of 0.80 or more; able to walk 150 meters in 6 minutes and maintain saturation 83% while on no more than 6 L/min supplemental oxygen. Patients are excluded from this study if they satisfy any of the following criteria: unable to undergo pulmonary function testing; evidence of significant obstructive lung disease or airway hyper-responsiveness; in the clinical opinion of the investigator, the patient is expected to need and be eligible for a lung transplant within 52 weeks of randomization; active infection; liver disease; cancer or other medical condition likely to result in death within 2 years; diabetes; pregnancy or lactation; substance abuse; personal or family history of long QT syndrome; other IPF treatment; unable to take study medication; withdrawal from other IPF trials. Patients are orally dosed with either placebo or an amount of a compound of a preferred embodiment (1 mg/day-1000 mg/day). The primary outcome variable will be the absolute change in percent predicted FVC from Baseline to Week 52. Patients will receive blinded study treatment from the time of randomization until the last patient randomized has been treated for 52 weeks. Physical and clinical laboratory assessments will be performed at defined intervals during the treatment duration, for example at weeks 2, 4, 8, 13, 26, 39, and 52. Pulmonary function, exercise tolerance, and shortness-of-breath will be assessed at defined intervals during the treatment duration, for example at weeks 13, 26, 39, and 52. A Data Monitoring Committee (DMC) will periodically review safety and efficacy data to ensure patient safety.

### Example Trial in SSc

The efficacy of treatment with a compound of a preferred embodiment compared with placebo in patients with systemic sclerosis (SSc) and the safety of treatment with a compound of a preferred embodiment compared with placebo in patients with SSc is assessed. The primary outcome variable is the absolute change in Modified Rodnan Skin Score (mRSS) from baseline to Week 48. Other possible end-points would include, but are not limited to: mortality, percentage of patients with treatment-emergent adverse events (AEs) and serious adverse events (SAEs), composite measurement of disease progression, and change in biomarkers (molecular and cellular markers of disease activity, such as C-reactive protein). Secondary outcome measures include, but are not limited to: Scleroderma Health Assessment Questionnaire (SHAQ) score; the Health Assessment Questionnaire Disability Index (HAQ-DI); Functional Assessment of Chronic Illness Therapy-Fatigue (FACIT) score; severity of pruritus as measured by a standardized scale, such as the 5-D Itch Scale; St. George's Respiratory Questionnaire (SGRQ) score; Tender Joint Count 28 (TCJ28); lung function parameters; standard vital signs (including blood pressure, heart rate, and temperature); electrocardiogram measurements (ECGs); laboratory tests (clinical chemistry, hematology, and urinalysis); pharmacokinetics (PK) measurements. Included in these measurements and in addition, clinical and biomarker samples, such as skin biopsies and blood (or serum and/or plasma), will also be collected prior to initiation of treatment. Additionally, patients eligible for this study include, but are not limited to, those patients that satisfy the following criteria: Patients at least 18 years of age; diagnosis of SSc according to the American College of Rheumatology (ACR) and European League Against Rheumatism (EULAR) Criteria, meeting criteria for active disease and with a total disease duration of less than or equal to 60 months; 10≤mRSS≤35. Patients are excluded from this study if they satisfy any of the following criteria: major surgery within 8 weeks prior to screening; scleroderma limited to area distal to the elbows or knees; rheumatic autoimmune disease other than SSc; use of any investigational, biologic, or immunosuppressive therapies, including intra-articular or parenteral corticosteroids within 4 weeks of screening. Patients are orally dosed with either placebo or an amount of a compound of a preferred embodiment (1 mg/day-1000 mg/day). The primary outcome variable will be the absolute change in mRSS \from Baseline to Week 48. Patients will receive blinded study treatment from the time of randomization until the last patient randomized has been treated for 48 weeks. Physical and clinical laboratory assessments will be performed at defined intervals during the treatment duration, such as Weeks 2, 4, 8, 12, 24, 36, and 48. Clinical and biomarker samples will also be collected at Week 48. A Data Monitoring Committee (DMC) will periodically review safety and efficacy data to ensure patient safety.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Although the invention has been described with reference to embodiments and examples, it should be understood that numerous and various modifications can be made without departing from the spirit of the invention. Accordingly, the invention is limited only by the following claims.

### REFERENCES CITED

1. U.S. Pat. No. 5,145,684
2. Goll et al. (2003). “The calpain system.” Physiol Rev 83(3):731-801.
3. Schad et al. (2002). “A novel human small subunit of calpains.” Biochem J 362(Pt 2):383-8.
4. Ravulapalli et al. (2009). “Distinguishing between calpain heterodimerization and homodimerization.” FEBS J 276(4):973-82.
5. Dourdin et al. (2001). “Reduced cell migration and disruption of the actin cytoskeleton in calpain-deficient embryonic fibroblasts.” J Biol Chem 276(51):48382-8.
6. Leloup et al. (2006). “Involvement of calpains in growth factor-mediated migration.” Int J Biochem Cell Biol 38(12):2049-63.
7. Janossy et al. (2004). “Calpain as a multi-site regulator of cell cycle.” Biochem Pharmacol 67(8):1513-21.
8. Santos et al. (2012). “Distinct regulatory functions of calpain 1 and 2 during neural stem cell self-renewal and differentiation.” PLoS One 7(3):e33468.
9. Miettinen et al. (1994). “TGF-beta induced transdifferentiation of mammary epithelial cells to mesenchymal cells: involvement of type I receptors.” J Cell Biol 127(6 Pt 2):2021-36.
10. Lamouille et al. (2014). “Molecular mechanisms of epithelial-mesenchymal transition.” Nat Rev Mol Cell Biol 15(3):178-96.
11. Pegorier et al. (2010). “Bone Morphogenetic Protein (BMP)-4 and BMP-7 regulate differentially Transforming Growth Factor (TGF)—B1 in normal human lung fibroblasts (NHLF)” Respir Res 11:85.

What is claimed is:

1. A compound having the structure of the formula I: 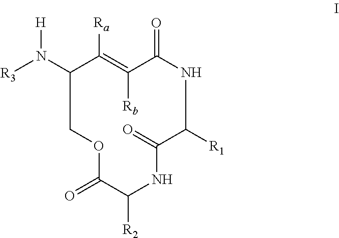
or a pharmaceutically acceptable salt thereof, wherein:
Rₐ and R_{b} are independently selected from —H, optionally substituted C₁₋₈ alkyl, and optionally substituted C₁₋₈ alkoxyalkyl;
R₃ is selected from the group consisting of —H, —COOR₃ₐ, —CON(R_{3b})₂, —COC(R₄)₂NH(R₅), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;
R₅ is selected from the group consisting of —H, —COOR₃, —CON(R_{3b})₂, —COC(R₆)₂NH(R₇), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;
R₇ is selected from the group consisting of —H, —COOR₃ₐ, —COR_{3b}, —COC(R₄)₂NH(R₅), optionally substituted C₁₋₄ alkyl, optionally substituted C₁₋₈ alkoxyalkyl, optionally substituted C₃₋₇ carbocyclyl, optionally substituted 5-10 membered heterocyclyl, optionally substituted C₆₋₁₀ aryl, and optionally substituted C₆ aryl(C₁-C₆)alkyl
R₁, R₂, R₄, and R₆ are independently selected from —H, optionally substituted C₁₋₄ alkyl, and optionally substituted C₁₋₈ alkoxyalkyl, optionally substituted C₆₋₁₀ aryl, optionally substituted 5-10 membered heteroaryl, optionally substituted C₃₋₁₀ carbocyclyl, optionally substituted C₂₋₈ alkenyl, optionally substituted aralkyl wherein the aryl ring in the aralkyl is further optionally substituted with one or more R₈, optionally substituted —O—C₁₋₆ alkyl, optionally substituted
—O C₂₋₆ alkenyl, and any natural or non-natural amino acid side chain;
R₈ is —OSi C₁₋₄ alkyl; and
R₃ₐ and R_{3b} are independently selected from —H, optionally substituted C₁₋₄ alkyl, optionally substituted C₃₋₇ carbocyclyl, optionally substituted aralkyl, optionally substituted C₂₋₁₀ alkenyl, optionally substituted 5-10 membered heterocyclyl, optionally substituted C₆₋₁₀ aryl, and optionally substituted 5-10 membered heteroaryl.

2. The compound of claim 1 having the structure of formula I-c: 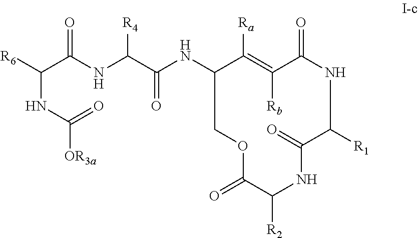
or a pharmaceutically acceptable salt thereof, wherein:
Rₐ and R_{b} are independently selected from —H and optionally substituted C₁₋₈ alkyl;
R₁ R₂, and R₄ are independently selected from —H, optionally substituted C₁₋₄ alkyl, optionally substituted C₆₋₁₀ aryl, optionally substituted aralkyl wherein the aryl ring is further substituted with one or more R₈, and any natural or non-natural amino acid side chain; and
R₃ₐ is selected from optionally substituted C₁₋₄ alkyl, optionally substituted aralkyl, and optionally substituted C₆₋₁₀ aryl.

3. The compound of any one of the claims 1 and 2, wherein R₃ₐ is selected from the group consisting of —H, optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

4. The compound of claim 3, wherein R₃ₐ is selected from the group consisting of tert-butyl, methyl, and benzyl.

5. The compound of claim 1 having the structure of formula I-d: 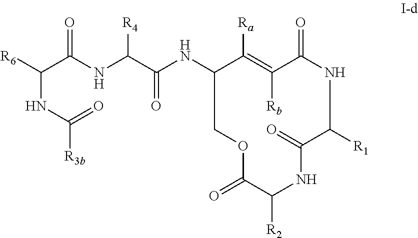
or a pharmaceutically acceptable salt thereof, wherein:
Rₐ and R_{b} are independently selected from —H and optionally substituted C₁₋₈ alkyl;
R₁ R₂, R₄, and R₆ are independently selected from —H, optionally substituted C₁₋₄ alkyl, optionally substituted C₆₋₁₀ aryl, optionally substituted aralkyl wherein the aryl ring is further substituted with one or more R, and any natural or non-natural amino acid side chain; and
R_{3b} is selected from optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

6. The compound of any one of the claims 1 and 5, wherein R_{3b} is selected from the group consisting of optionally substituted C₁₋₄ alkyl, optionally substituted aralkyl, optionally substituted C₂₋₁₀ alkenyl, and optionally substituted C₆₋₁₀ aryl.

7. The compound of claim 6, wherein R_{3b} is selected from the group consisting of methyl, —CH₂CH═CH(CH₂)₅CH₃, and benzyl.

8. The compound of any one of the claims 1-7, wherein R₆ is selected from the group consisting of —H, optionally substituted C₁₋₄ alkyl, optionally substituted C₆₋₁₀ aryl, optionally substituted C₃₋₁₀ carbocyclyl, optionally substituted C₂₋₈ alkenyl, optionally substituted aralkyl wherein the aryl ring is further substituted with one or more R₈.

9. The compound of claim 8, wherein R₆ is selected from the group consisting of —H, optionally substituted C₁₋₄ alkyl, and optionally substituted aralkyl wherein the aryl ring is further substituted with one or more R₈.

10. The compound of claim 9, wherein R₆ is selected from the group consisting of methyl, isopropyl, isobutyl, benzyl, and p-hydroxybenzyl, and p-methoxybenzyl.

11. The compound of claim 1 having the structure of formula I-b: 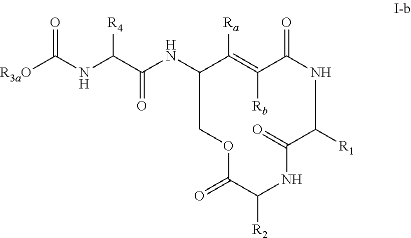
or a pharmaceutically acceptable salt thereof, wherein:
Rₐ and R_{b} are independently selected from —H and optionally substituted C₁₋₈ alkyl;
R₁ R₂, and R₄ are independently selected from —H, optionally substituted C₁₋₄ alkyl, optionally substituted C₆₋₁₀ aryl, optionally substituted aralkyl wherein the aryl ring is further substituted with one or more R₈, and any natural or non-natural amino acid side chain; and
R₃ₐ is selected from optionally substituted C₁₋₄ alkyl, optionally substituted aralkyl, and optionally substituted C₆₋₁₀ aryl.

12. The compound of claim 11, wherein R₃ₐ is selected from the group consisting of —H, optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

13. The compound of claim 12, wherein R₃, is selected from the group consisting of tert-butyl, methyl, and benzyl.

14. The compound of any one of the claims 1-13, wherein R₄ is selected from the group consisting of —H, optionally substituted C₁₋₄ alkyl, optionally substituted C₆₋₁₀ aryl, optionally substituted C₃₋₁₀ carbocyclyl, optionally substituted C₂₋₄ alkenyl, optionally substituted aralkyl wherein the aryl ring is further substituted with one or more R₈.

15. The compound of claim 14, wherein R₄ is selected from the group consisting of —H, optionally substituted C₁₋₄ alkyl, and optionally substituted aralkyl wherein the aryl ring is further substituted with one or more R₈.

16. The compound of claim 15, wherein R₄ is selected from the group consisting of methyl, isopropyl, isobutyl, benzyl, and p-hydroxybenzyl, and p-methoxybenzyl.

17. The compound of claim 1 having the structure of formula I-a: 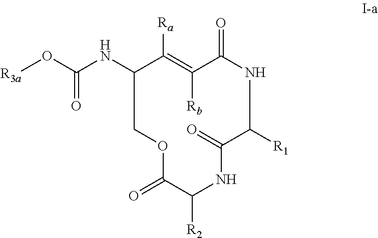
or a pharmaceutically acceptable salt thereof, wherein:
Rₐ and R_{b} are independently selected from —H and optionally substituted C₁₋₈ alkyl;
R₁ and R₂ are independently selected from —H, optionally substituted C₁₋₄ alkyl, optionally substituted C₆₋₁₀ aryl, optionally substituted aralkyl wherein the aryl ring is further substituted with one or more R₈, and any natural or non-natural amino acid side chain; and
R₃ₐ is selected from optionally substituted C₁₋₄ alkyl, optionally substituted aralkyl, and optionally substituted C₆₋₁₀ aryl.

18. The compound of claim 17, wherein R₃ is selected from the group consisting of —H, —COOR₃ₐ, —CON(R_{3b})₂, optionally substituted C₁₋₄ alkyl, optionally substituted C₃₋₇ carbocyclyl, optionally substituted C₆₋₁₀ aryl, and optionally substituted C₆₋₁₀ aryl(C₁-C₆)alkyl.

19. The compound of claim 18, wherein R₃ is selected from the group consisting of —H, —COOR₃ₐ, —CON(R_{3b})₂, optionally substituted C₁₋₄ alkyl, and optionally substituted C₆₋₁₀ aryl(C₁-C₆)alkyl.

20. The compound of any one of claims 17-19, wherein R₃ₐ is selected from the group consisting of —H, optionally substituted C₁₋₄ alkyl, optionally substituted C₃₋₇ carbocyclyl, optionally substituted aralkyl, optionally substituted C₂₋₁₀ alkenyl, and optionally substituted C₆₋₁₀ aryl.

21. The compound of claim 20, wherein R₃ₐ is selected from the group consisting of tert-butyl, methyl, and benzyl.

22. The compound of any one of the claims 18 and 19, wherein R_{3b} is selected from the group consisting of optionally substituted C₁₋₄ alkyl, optionally substituted aralkyl, optionally substituted C₂₋₁₀ alkenyl, and optionally substituted C₆₋₁₀ aryl.

23. The compound of claim 22, wherein R_{3b} is selected from the group consisting of methyl, —CH₂CH═CH(CH₂)₅CH₃, and benzyl.

24. The compound of any one of the claims 1-23, wherein R₁ and R₂ are independently selected from —H, optionally substituted C₁₋₄ alkyl, optionally substituted C₆₋₁₀ aryl, optionally substituted C₃₋₁₀ carbocyclyl, optionally substituted C₂₋₈ alkenyl, optionally substituted aralkyl wherein the aryl ring is further optionally substituted with one or more R₈.

25. The compound of claim 24, wherein R₁ and R₂ are independently selected from —H, optionally substituted C₁₋₄ alkyl, and optionally substituted aralkyl wherein the aryl ring is further optionally substituted with one or more R₈.

26. The compound of claim 25, wherein R₁ and R₂ are independently selected from the group consisting of methyl, isopropyl, isobutyl, benzyl, and p-hydroxybenzyl, and p-methoxybenzyl.

27. The compound of any one of the claims 1-25, wherein R₈ is —OSi C₁₋₄ alkyl.

28. The compound of claim 27, wherein R₈ is selected from the group consisting of OSiMe₃ and OSi^{t}BuMe₂.

29. The compound of any one of the claims 1-28, wherein Rₐ and R_{b} are independently selected from —H and optionally substituted C₁₋₈ alkyl.

30. The compound of claim 1-29 wherein Rₐ and R_{b} are —H.

31. The compound of claim 1, having the structure selected from the group consisting of: 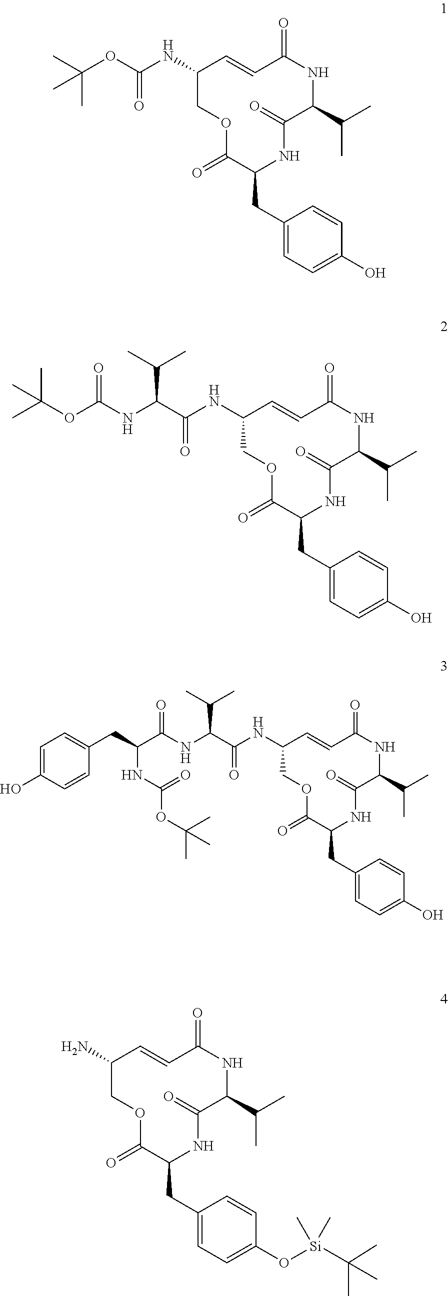 [SMILES: CC(C)[C@@H]1NC(=O)/C=C/[C@@H](NC(=O)OC(C)(C)C)COC(=O)[C@H](Cc2ccc(O)cc2)NC1=O ; CC(C)[C@@H]1NC(=O)/C=C/[C@@H](NC(=O)[C@@H](NC(=O)OC(C)(C)C)C(C)C)COC(=O)[C@H](Cc2ccc(O)cc2)NC1=O ; CC(C)[C@@H]1NC(=O)/C=C/[C@@H](NC(=O)[C@@H](NC(=O)[C@H](Cc2ccc(O)cc2)NC(=O)OC(C)(C)C)C(C)C)COC(=O)[C@H](Cc2ccc(O)cc2)NC1=O ; CC(C)[C@@H]1NC(=O)/C=C/[C@@H](N)COC(=O)[C@H](Cc2ccc(O[Si](C)(C)C(C)(C)C)cc2)NC1=O] 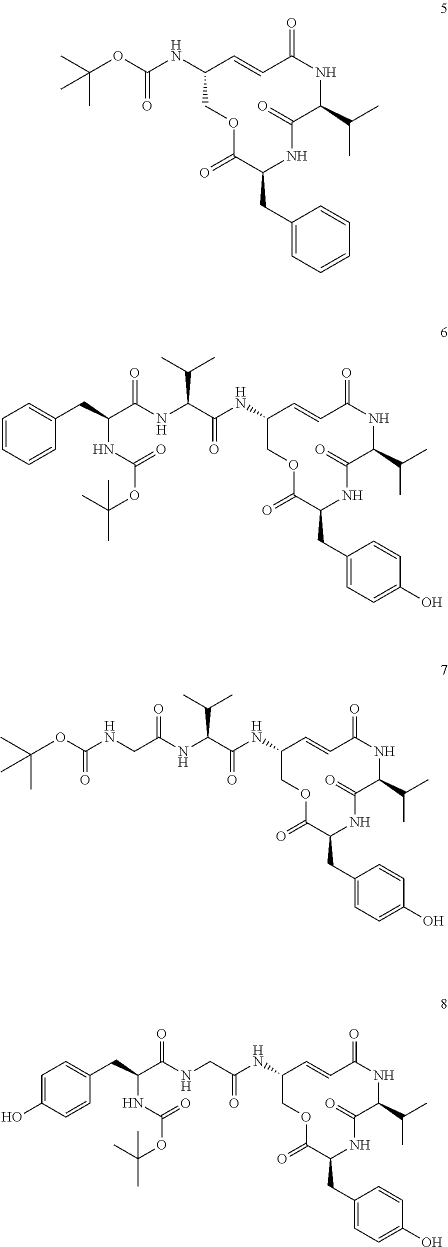 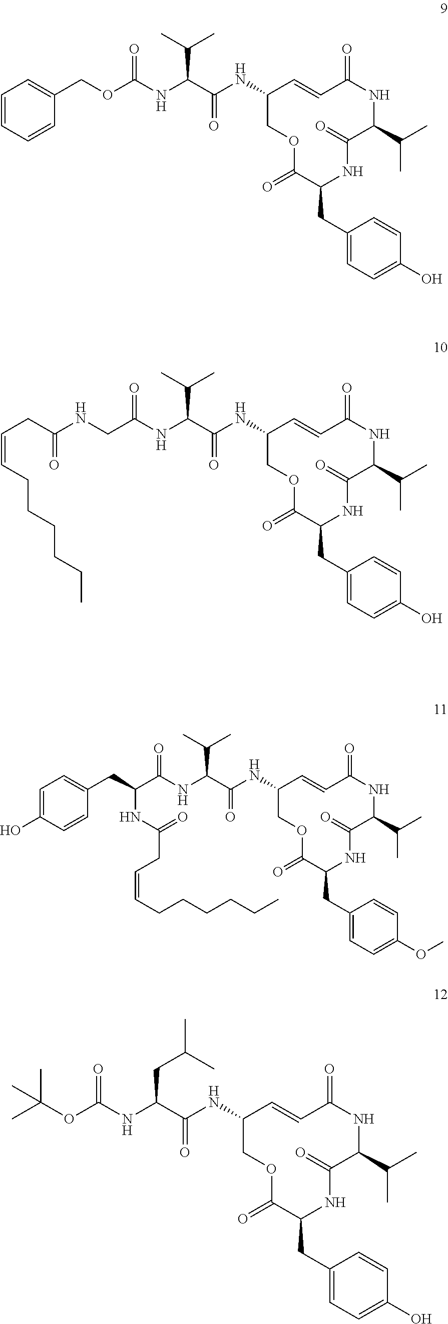 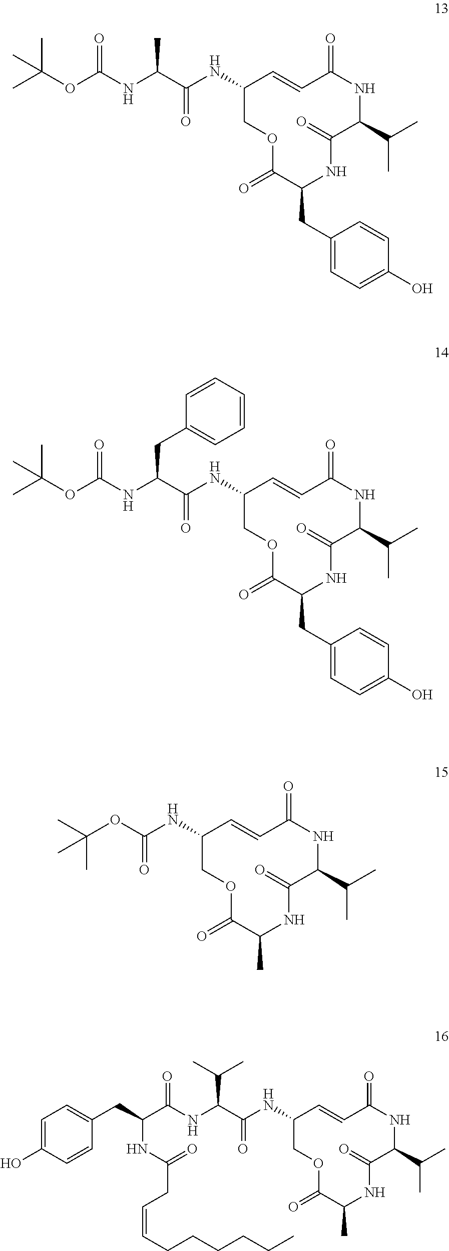 [SMILES: CC(C)[C@@H]1NC(=O)/C=C/[C@@H](NC(=O)[C@H](C)NC(=O)OC(C)(C)C)COC(=O)[C@H](Cc2ccc(O)cc2)NC1=O ; CC(C)[C@@H]1NC(=O)/C=C/[C@@H](NC(=O)[C@H](Cc2ccccc2)NC(=O)OC(C)(C)C)COC(=O)[C@H](Cc2ccc(O)cc2)NC1=O ; CC(C)[C@@H]1NC(=O)/C=C/[C@@H](NC(=O)OC(C)(C)C)COC(=O)[C@H](C)NC1=O ; CCCCCC/C=C\CC(=O)N[C@@H](Cc1ccc(O)cc1)C(=O)N[C@H](C(=O)N[C@@H]1/C=C/C(=O)N[C@@H](C(C)C)C(=O)N[C@@H](C)C(=O)OC1)C(C)C] 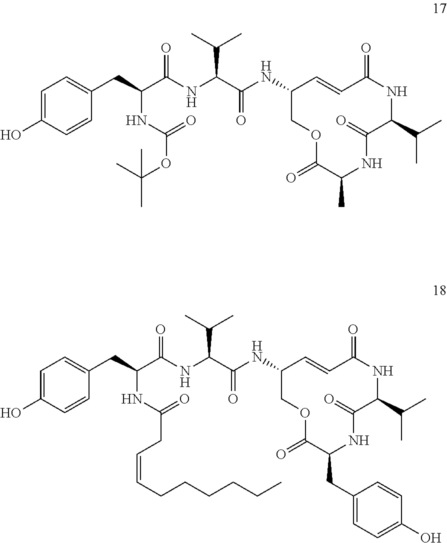 [SMILES: CC(C)[C@@H]1NC(=O)/C=C/[C@@H](NC(=O)[C@@H](NC(=O)[C@H](Cc2ccc(O)cc2)NC(=O)OC(C)(C)C)C(C)C)COC(=O)[C@H](C)NC1=O ; CCCCCC/C=C\CC(=O)N[C@@H](Cc1ccc(O)cc1)C(=O)N[C@H](C(=O)N[C@@H]1/C=C/C(=O)N[C@@H](C(C)C)C(=O)N[C@@H](Cc2ccc(O)cc2)C(=O)OC1)C(C)C]
and pharmaceutically acceptable salts thereof.

32. A pharmaceutical composition comprising a therapeutically effective amount of a compound of any one of claims 1-31 and a pharmaceutically acceptable excipient.

33. A method of treating fibrotic disease or a secondary disease state or condition thereof, comprising administering to a subject in need thereof, a compound according to any one of claims 1-31.

34. The method of claim 33, wherein the disease is selected from the group consisting of liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis.

35. The method of claim 33, wherein the treatment decreases the expression level and/or activity of a calpain.

36. The method of claim 35, wherein the calpain is CAPN1, CAPN2, or CAPN9.

37. The method of claim 33, wherein the treatment inhibits myofibroblast differentiation or treats a disease associated with myofibroblast differentiation.

38. The method of claim 33, wherein the treatment inhibits Fibroblast-to-Myofibroblast Transition (FMT).

39. The method of claim 33, wherein the treatment inhibits Epithelial to Mesenchymal Transition or Endothelial to Mesenchymal Transition.

40. The method of claim 39 wherein the myofibroblast differentiation is a TGFβ-mediated myofibroblast differentiation.

41. The method of claim 33, wherein the fibrotic disease is a cancer.

42. The method of claim 41, wherein the cancer is a cancer of epithelial origin.

43. The method of claim 42, wherein the cancer of epithelial origin is selected from the group consisting of breast cancer, basal cell carcinoma, adenocarcinoma, gastrointestinal cancer, lip cancer, mouth cancer, esophageal cancer, small bowel cancer, stomach cancer, colon cancer, liver cancer, brain, bladder cancer, pancreas cancer, ovary cancer, cervical cancer, lung cancer, skin cancer, prostate cancer, and renal cell carcinoma.

44. The method of claim 33, wherein the fibrotic disease is stiff skin syndrome (SKS).

45. The method of claim 33, wherein the compound is of Formula I.

46. The method of claim 33, wherein the subject is a mammal.

47. The method of claim 33, wherein the subject is a human.

48. The method of claim 33 wherein the route of administration is selected from the group consisting of: enteral, intravenous, oral, intraarticular, intramuscular, subcutaneous, intraperitoneal, epidural, transdermal, and transmucosal.

49. The method of claim 33, wherein the administration is intravenous.

50. A method of inhibiting myofibroblast differentiation comprising contacting a cell with a compound of anyone of claims 1-31.

51. The method of claim 50, wherein the cell is in a fibrotic tissue.

52. The method of claim 50, wherein the cell is in a cancerous tissue.

53. The method of claim 50, wherein the cell is in a tissue with high TGFβ signaling.

54. A method for inhibiting calpain, the method comprising contacting a compound of any one of claims 1-31 with a CAPN1, CAPN2, and/or CAPN9 enzyme residing inside a subject.
